(19) United States
(12) Patent Application Publication
(10) Pub. No.: US 2025/0297290 A1
(43) Pub. Date: Sep. 25, 2025

(54) SEQUENCE FOR PROTEIN DECAY

(71) Applicants: Lars M. BLANK, Dortmund (DE); Birgitta E. EBERT, Queensland (AU); Hao GUO, Aachen (DE); Jochen FÖRSTER, Copenhagen (DK); Kerstin WALTER, lngelheim am Rhein (DE); Jerome MAURY, Kirke Såby (DK); Christoph KNUF, Kalundborg (DK); Simo JACOBSEN, Copenhagen (DK)

(72) Inventors: Lars M. BLANK, Dortmund (DE); Birgitta E. EBERT, Queensland (AU); Hao GUO, Aachen (DE); Jochen FÖRSTER, Copenhagen (DK); Kerstin WALTER, lngelheim am Rhein (DE); Jerome MAURY, Kirke Såby (DK); Christoph KNUF, Kalundborg (DK); Simo JACOBSEN, Copenhagen (DK)

(21) Appl. No.: 18/709,148

(22) PCT Filed: Nov. 11, 2022
(86) PCT No.: PCT/EP2022/081589

§ 371 Date: May 10, 2024

(30) Foreign Application Priority Data
Nov. 11, 2021 (EP) 21207664.0

(51) Int. Cl. C12P 7/00; C07K 7/08; C12N 9/02; C12N 9/04; C12N 9/90; C12N 15/81; C12P 5/00

(52) U.S. Cl. CPC C12P 7/00; C07K 7/08; C12N 9/0006; C12N 9/0091; C12N 9/90; C12N 15/81; C12P 5/007; C12Y 101/01034; C12Y 116/01008; C12Y 504/99007; C07K 2319/50; C12N 2800/102; C12N 2830/002

(57) ABSTRACT

The present invention relates to an expression cassette encoding a fusion protein comprising a nucleotide sequence encoding an amino acid sequence shown in SEQ ID NO: 1 or a fragment thereof, which directs protein decay, or encoding an amino acid sequence which is at least 60% identical to the amino acid sequence which directs protein decay; and also comprising a nucleotide sequence encoding a protein of interest, wherein the nucleotide sequences are fused together in frame. Further, the present invention relates to a vector comprising the expression cassette, a host cell comprising the expression cassette or a host cell comprising the vector which comprises the expression cassette. Additionally, the present invention relates to a method for the production of a triterpenoid using the host cell comprising the expression cassette of the present invention.

### CROSS-REFERENCE TO RELATED APPLICATIONS

The present application claims the benefit of priority of EP-A Patent Application No. 21 207 664 filed 11 Nov. 2021, the content of which is hereby incorporated by reference in its entirety for all purposes.

### TECHNICAL FIELD OF THE INVENTION

The present invention relates to an expression cassette encoding a fusion protein comprising a nucleotide sequence encoding an amino acid sequence shown in SEQ ID NO: 1 or a fragment thereof, which directs protein decay, or encoding an amino acid sequence which is at least 60% identical to the amino acid sequence which directs protein decay; and also comprising a nucleotide sequence encoding a protein of interest, wherein the nucleotide sequences are fused together in frame and wherein the fragment is at least 18 amino acids long. Further, the present invention relates to a vector comprising the expression cassette, a host cell comprising the expression cassette or a host cell comprising the vector which comprises the expression cassette. Additionally, the present invention relates to a method for the production of a triterpenoid using the host cell comprising the expression cassette of the present invention.

### BACKGROUND OF THE INVENTION

A gradual reduction of protein level can be advantageous in the production of certain metabolites. Especially when tuning down enzymes of metabolic pathways which are directly linked to cell survival, a complete genetic knockout may be impossible or less preferably. Little is known about how amino acid sequences function as degradation signals, how they influence protein stability or induce proteolysis. For some application, such as metabolic engineering, it may be desirable to influence a biosynthesis pathway by modulating protein activity, e.g. via its half-life or via gradual degradation. The later may be accomplished by equipping proteins involved in a biosynthesis pathway with a degradation signal. Knuf et al. 2014 for example focuses on the action of such degron strategy for controlling the pools of intermediates of the mevalonate pathway around 2,3-oxidosqualene, thereby down-regulating the expression of enzymes involved in such pathway. Any particular degron sequence is not disclosed at all.

Interesting biosynthesis pathways are those for cyclic triterpenoid synthesis. Due to their biological activity, cyclic triterpenoids resulting from the sterol synthesis pathways are of major interest for the pharmaceutical industry. Thus, an increase in the synthetic yield represents a great financial interest. However, it is challenging to obtain triterpenoids from natural sources because of the limited supply and complicated extraction processes. In addition, chemical synthesis of these compounds is not a feasible approach because of their complex structure (Chang and Keasling 2006). Microbial production is an alternative, and this strategy possesses several advantages as compared to plant-based production, such as short producing periods, and independence of climate. S. cerevisiae has been successfully employed for the production of triterpenoids, containing, for example, a genetically manipulated (S)-3-hydroxy-3-methylglutaryl-coenzyme-A (HMG-COA) reductase (Polakowski, Stahl and Lang 1998) and a dysfunctional steryl acyltransferase (WO2012/116783). In this context, Kalaivani and Sarma 2017 also mentions different manipulation methods inside the sterol pathway for the production of terpenes and terpenoids in S. cerevisiae, such as overexpressing ERG20 or ERG9 enzyme, or inter alia manipulating (point mutation) ERG20 (and also ERG9), which fuels farnesyl pyrophosphate (FPP) and geranyl pyrophosphate (GPP) synthesis reactions (see Table 3). A different prior art document regarding the down-regulation of ERG20 by applying a N-degron-dependent protein strategy is mentioned in Peng et al. 2018. As degron, any one of degron K3K15, KN113 or KN119 as disclosed in Suzuki and Varshavsky 1999 in more detail was applied for the particular fusion protein.

Since ergosterol is an essential component of the plasma membrane, a knockout of the ERG7 gene in S. cerevisiae resulted in an exhaustion of downstream sterols, which is an infeasible approach for an industrial process. In this case, ergosterol needs to be supplemented to the growth medium. Hence, the reduced expression of ERG7 was used to redirect the carbon flux towards the production of triterpenoids. Repression of ERG7 by a replacement of the native promoter with the copper-regulated promoter PCTR resulted in a high 2,3-oxidosqualene accumulation (ca. 30% (g/g cell dry weight)). However, an additional expression of a lupeol synthase did not result in comparable high lupeol titers (2%) (Broker et al., 2018). Similarly, TetR-TetO based gene regulation was used to manipulate the transcriptional efficiency of ERG7, which led to a 31% increase in the volumetric titer of protopanaxadiol despite a growth deficit of about 21% (Zhao et al., 2019). These results suggested that downregulation of ERG7 transcription has a minor effect on improving triterpenoid synthesis. The resulting yields are however not satisfying, and any improvement in the production yields would be highly desirable. This is also true for many other biosynthesis pathways. For example WO2017/004022 discloses an engineered fusion protein comprising also a degron sequence which is fused to a polypeptide of interest, which may be fused to a “small-molecule assisted shutoff” (SMASh) tag via a HCV non-structural protein 3 (NS3) protease recognition site. Such degron however refers to an amino acid sequence which is as follow “PITKIDTKYIMTCMSADLEVVTSTWVLVGGVLAALAAYCLST” or a variant thereof comprising a sequence having at least about 80% sequence identity thereto.

Thus, there remains a need for strategies to increase the yield of desired products of biosynthesis pathways, e.g. for the production of cyclic triterpenes.

The technical problem underlying the present application is thus to comply with these needs. The technical problem is solved by providing the embodiments reflected in the claims, described in the description and illustrated in the examples and figures that follow.

### SUMMARY OF THE INVENTION

The present invention relates in a first aspect to an expression cassette encoding a fusion protein comprising a) a nucleotide sequence encoding (i) an amino acid sequence shown in SEQ ID NO: 1 or a fragment thereof which directs protein decay, or encoding (ii) an amino acid sequence which is at least 60% identical to the amino acid sequence of (i) which directs protein decay; and comprising b) a nucleotide sequence encoding a protein of interest, wherein nucleotide sequence a) and b) are fused together in frame and wherein the fragment is at least 18 amino acids long. The fusion of these nucleotide sequences may lead to a gradual level reduction of the translated fusion protein.

The present invention may further comprise the expression cassette as described elsewhere herein, wherein the amino acid sequence as defined elsewhere herein in a) is located at the N-terminus, at the C-terminus or within the protein of interest as defined elsewhere herein in b).

Additionally, the present invention may envisage the expression cassette as described elsewhere herein, wherein it further comprises c) one or more nucleotide sequence(s) fused to the 5′- and/or 3′-end of the described nucleotide sequences a) and/or b). Further, said expression cassette may comprise d) one or more nucleotide sequence(s) which is/are comprised in the nucleotide sequence a), b) or c) and which is fused in frame with the nucleotide sequences of a), b) and/or c).

The present invention may also comprise the expression cassette as described elsewhere herein, wherein the nucleotide sequence of a) is shown in SEQ ID NO: 2.

Also comprised by the present invention is the expression cassette as described elsewhere herein, wherein the level of the fusion protein gradually reduces when expressed in a cell in comparison to a cell which expresses the protein of interest.

The present invention may also encompass the expression cassette as described elsewhere herein, wherein said nucleotide sequence of d) comprises at least 3 nucleotides and encodes a heterologous polypeptide, wherein said heterologous polypeptide is a linker, tag and/or cleavable site for a protease.

Further, the present invention may envisage the expression cassette as described elsewhere herein, wherein a constitutively active or inducible expression control sequence is operatively linked with the expression cassette, wherein the inducible expression control sequence is inducible preferably by temperature, light, small molecules or the expression of another protein.

Additionally, the present invention may comprise the expression cassette as described elsewhere herein, wherein said nucleotide sequence of b) encodes a polypeptide selected from a group consisting of enzymes, receptors, receptor ligands, antibodies, lipocalins, hormones, inhibitors, membrane proteins, membrane-associated proteins, peptidic toxins, and peptidic antitoxins.

Preferably, the enzyme as described elsewhere herein is a lanosterol synthase, even more preferably the enzyme is Erg7p as shown in SEQ ID NO: 3.

Further, the present invention may comprise the expression cassette as described elsewhere herein further comprising a nucleotide sequence encoding a selection marker which preferably confers resistance against an antibiotic or anti-metabolite.

The present invention relates in a second aspect to a vector comprising the expression cassette as defined elsewhere herein.

The present invention relates in a third aspect to a host cell comprising the expression cassette or the vector as defined elsewhere herein.

Preferably, the protein of interest comprised by the expression cassette which is comprised by the host cell as defined elsewhere herein is a lanosterol synthase. Even more preferably, the protein of interest comprised by the expression cassette which is comprised by the host cell as defined elsewhere herein is Erg7p as shown in SEQ ID NO: 3. Most preferably, the lanosterol synthase comprised by the expression cassette is encoded by the nucleotide sequence as shown in SEQ ID NO: 4.

The host cell as described herein in the present invention may be a bacterial, a mammalian or a fungal host cell, preferably said host cell of the present invention is a yeast host cell.

The host cell as described herein may further not express one or more sterol acyltransferases, preferably: (i) Are1p as shown in SEQ ID NO: 15 and/or (ii) Are2p as shown in SEQ ID NO: 16.

The present invention may further comprise the host cell as defined herein which further expresses one or more of the following proteins: (i) a truncated HMG-COA reductase; (ii) an oxidosqualene cyclase; (iii) a cytochrome P450 monooxygenase; (iv) a cytochrome P450 reductase; (v) a sterol acyltransferase.

In a fourth aspect the present invention relates to a method for the production of a triterpenoid comprising culturing a host cell as defined elsewhere herein under conditions which allow the production of a triterpenoid; and harvesting the triterpenoid produced by said host cell.

### BRIEF DESCRIPTION OF THE FIGURES

FIG. 1 shows a schematic of the native mevalonate and ergosterol pathway and the heterologous betulinic acid pathway and the enzymes involved. 2,3-oxidosqualene is usually transformed to lanosterol by Erg7p enzyme. A reaction competing for 2,3-oxidosqualene is catalysed by lupeol synthase from Betula platyphylla (OSCBPW), which forms lupeol. Lupeol is further oxidised to betulinic acid. Arrows with dashed lines represent lumped reactions.

FIG. 2 shows a schematic representation of the ERG7 decay variants (A), specific 2,3-oxidosqualene titres in strains with ERG7 decay variants (B) and (C). The bleR cassette was used to replace different parts of the sequence downstream of the frameshift ERG7 in Simo1575. The locus for integration was defined by sequences flanking the bleR cassette that were homologous to the targeted site. The sequence (tGFP-cODC1-TDegF-RFP) in Simo1575 was completely or partially replaced by a bleR cassette, yielding Simo1575-gt-ERG7, Simo1575-m-ERG7, Simo1575-t-ERG7 and Simo1575-o-ERG7 strain, respectively. The Simo1575+ERG7 strain was generated by the replacement of the frameshift ERG7 mutant and the downstream tGFP-cODC1-TDegF-RFP construct with the yeast's native ERG7 gene in Simo1575. Strains were cultivated in YPD medium with 2% glucose for 72 h; error bars represent the standard deviation of three biological replicates; CDW, cell dry weight.

FIG. 3 shows the production of triterpenoids analysed by HPLC-CAD in strains with ERG7 decay variants. The recombinant strain BA6+ERG7 was generated from BA6 by the replacement of the frameshift ERG7 mutant and the degron tag with the native ERG7. Both strains were grown in WM8+medium for 72 h; error bars represent the standard deviation of three biological replicates.

FIG. 4 shows the intracellular concentration of lanosterol, zymosterol, and ergosterol in Simo1575 and an isogenic strain in which the frameshift ERG7 mutant and downstream tGFP-cODC1-TDegF-RFP had been replaced with the native ERG7 gene (Simo1575+ERG7). Both strains were cultivated in YPD medium for 72 h. Sterol analytics was carried out by GC-MS analysis; error bars represent the standard deviation of three biological replicates; CDW, cell dry weight.

FIG. 5 shows the triterpen(e/oid) production performance of the ERG7 decay variants 102-tm, 102-tmreerg7, and Simo1575 for squalene and 2,3-oxidosqualene. Volumetric (A) and specific (B) squalene titre in engineered strains 102-tm, 102-tm-reerg7, and Simo1575. Volumetric (C) and specific (D) 2,3-oxidosqualene titre in the engineered strains 102-tm, 102-tm-reerg7 and Simo1575. All strains were cultivated in YPD medium with 2% glucose for 72 h; error bars represent the standard deviation of three biological replicates; CDW, cell dry weight.

FIG. 6 shows the triterpenoid production performance of strains 102-tm-OEW, 102-tm-reeng7-OEW and Simo1575-OEW for lupeol. (A) Volumetric and (B) volumetric lupeol titre, and (C) specific lupeol titre in strain 102-tm-OEW, 102-tm-reerg7-OEW, and Simo1575-OEW. All strains were cultivated in YPD medium with 2% glucose for 72 h; error bars represent the standard deviation of three replicates; CDW, cell dry weight.

FIG. 7 shows the results for the production of the lupane-type triterpenoids betulinic acid, betulin, betulin aldehyde, lupeol, 2,3-oxiosqualene, and total triterpenoids in mg/L. (A) Triterpenoid titres in strains 102-tm-LP, 102-tm-reeng7-LP and Simo1575-LP with a single-copy integration of OEW (lupeol synthase) and CYP716A15; (B) triterpenoid titres in strains 102-tm-2LP and 102-tm-reeng7-LP with chromosomal integration of two copies of OEW (lupeol synthase) and CYP716A15. All strains were cultivated in YPD medium with 2% glucose for 72 h; error bars represent the standard deviation of three biological replicates.

FIG. 8 shows the results for the production of the lupane-type triterpenoids betulinic acid, betulin, betulin aldehyde, lupeol, 2,3-oxiosqualene, and total triterpenoids in mg/L. Triterpenoid titres in strains 102-tm-reerg7-are1-2LP1 and 102-tm-reerg7-are1-2LP2 with a double-copy integration of OEW (lupeol synthase) and a single-copy integration of Are1.

FIG. 9 shows (A) the results for the strains 102-tm-2BP, and 102-tm-reeng7-2BP containing two gene copies of AaBAS (β-amyrin synthase) and CYP716A15 (P450 monooxygenase) for the oleanane-type triterpenoids β-amyrin, erythrodiol, oleanolic aldehyde, oleanolic acid, and total oleanane-type triterpenoids in mg/L and (B) the results of 102-tm-2PP, and 102-tm-reeng7-2PP containing two gene copies of P450 monooxygenase for dammarenediol II, protopanaxadiol and total dammarane-type triterpenoids. All strains were cultivated in WM8+ medium with 5% glucose for 72 h; error bars represent the standard deviation of three replicates.

### DETAILED DESCRIPTION OF THE INVENTION

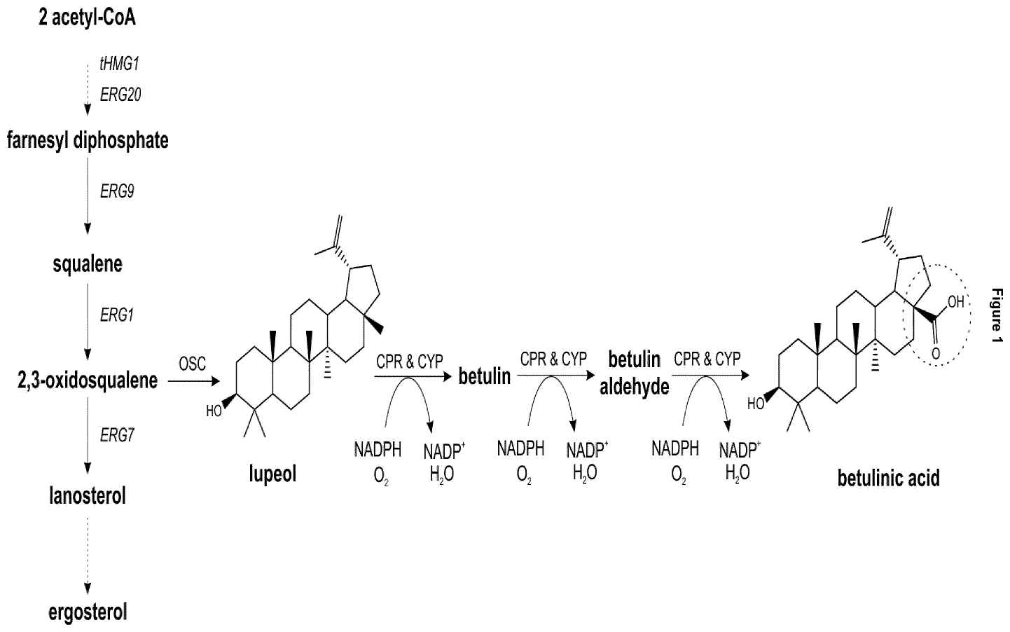

Usually, fluxes in biosynthetic pathways are redirected into a desired direction, e.g. by blocking the pathway at a desired (intermediate) product, thereby achieving an accumulation of the (intermediate) product which may be the precursor of a then-desired (end) product. In a next step, by either introducing additional copies of genes or overexpressing such genes encoding proteins which process accumulated (intermediate) products the flux is redirected into the desired direction. The present inventors, with the aim of producing triterpenoids in yeast, manipulated the ergosterol biosynthesis pathway, which concerts Acetyl-CoA via multiple steps into ergosterol, in order to achieve accumulation of 2,3-oxidosqualene (see FIG. 1). 2,3-oxidosqualene is the precursor for triterpenoids. Accordingly, the present inventors blocked the ergosterol biosynthesis pathway at the step which converts 2,3-oxidosqualene into lanosterol (see FIG. 1). This step is achieved in yeast and other organisms by a lanosterol synthase, encoded by the ERG7 gene in yeast. Blocking a biosynthetic pathway is usually achieved by reducing the expression level or complete inactivation of the gene encoding the protein which effects conversion of the desired (intermediate) product, thereby resulting in its accumulation. However, in contrast to the usual procedure, the present inventors decided to refrain from a classical inactivation on gene level and equipped the Erg7p with a degron sequence for decreasing its stability and lowering lanosterol synthase activity.

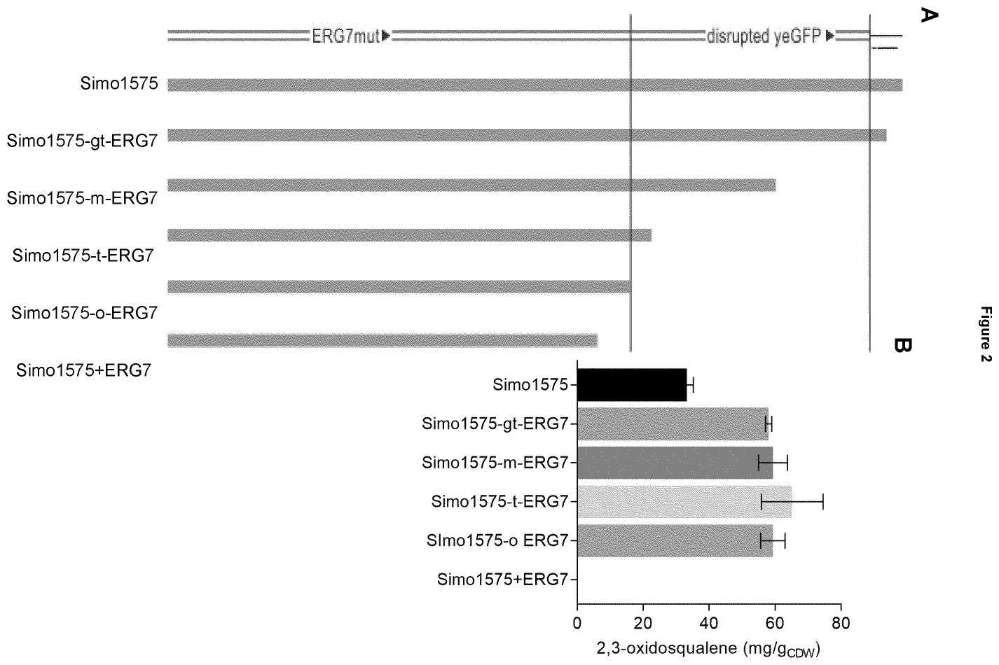

Much to their surprise, the present inventors observed that it was not the ERG7-degron equipped yeast strain which accumulated most of the desired 2,3-oxidosqualene, but a different yeast strain, designated as Simo1575. This strain accumulated double the amount of 2,3-oxidosqualene compared with all other tested clones (see FIG. 2) and hence attracted the present inventors' interest. It turned out that this strain somehow acquired a frameshift mutation close to the 3′-end of ERG7 which prolonged yeast Erg7p at amino acid position 728 for 31 amino acids. Accordingly, the last three amino acids of the 731 amino acid long wildtype Erg7p are replaced and Erg7p is prolonged for 28 amino acids. Given the fact that the Simo1575 yeast strain carrying the afore-described frameshift mutation has a reduced level of ergosterol renders it plausible that Erg7p is degraded to such an extent that the ergosterol synthesis in Simo1575 is limited and 2,3-oxidosqualene accumulates due to the reduced activity of lanosterol synthase encoded by ERG7.

The present inventors, rebuilt the Simo1575 yeast strain by removing the degron part and by merely expressing ERG7 carrying the frameshift which replaces the last three amino acids at the C-terminus and extends Erg7p for another 28 amino acids. It turned out that the rebuilt Simo1575-o-ERG7 yeast strain showed an even slightly increased accumulation of 2,3-oxidosqualene in comparison to the “original” frameshifted Simo1575 strain (see FIG. 2). Further variants of the “original” frameshifted Simo1575 yeast strain which also carry the frameshift, but different parts of the original degron part (e.g. Simo 1575-m-ERG7 or Simo1575-gt-ERG7) also showed increased accumulation of 2,3-oxidosqualene in comparison to the “original” frameshifted Simo1575 strain (see FIG. 2). The rebuilt Simo1575 yeast strain Simo-t-ERG7 and the variants Simo1575-m-ERG7 and Simo1575-gt-ERG7 share as common feature the frameshift which results in a “mutant” Erg7p, in which the last three wildtype amino acids at the C-terminus are replaced and Erg7p is extended. Given the fact that Simo1575-o-ERG7, in contrast to Simo1575-t-ERG7, Simo1575-m-ERG7 or Simo1575-gt-ERG7, does not have additional nucleotide sequences in the 3′-region flanking the frameshifted ERG7 gene, it is plausible that the frameshift resulting in an extension of Erg7p is causative for the phenotype, and an increased accumulation of 2,3-oxidosqualene and reduced level of ergosterol due to insufficient activity of lanosterol synthase encoded by the frameshifted ERG7 gene. Hence, the 31 amino acid sequence resulting from the frameshift in the 3′-region of ERG7 close to the wildtype stop codon seem to direct protein decay.

To this end, the present inventors found a novel amino acid sequence which is used for directing protein decay, i.e. they found a novel decay-tag (DT). This particular amino acid sequence/the novel decay-tag refers to the so-called “decay sequence” as mentioned in the present invention. Such a decay sequence may have versatile applications, e.g. for in vivo manipulation of protein abundance or activity by influencing a protein's half-life or leading to protein degradation.

Accordingly, the present invention relates in a first aspect to an expression cassette comprising a) a nucleotide sequence encoding (i) an amino acid sequence shown in SEQ ID NO: 1 or a fragment thereof which directs protein decay, or encoding (ii) an amino acid sequence which is at least 60% identical to the amino acid sequence of (i) which directs protein decay; and also comprising b) a nucleotide sequence encoding a protein of interest, wherein nucleotide sequence a) and b) are fused together in frame and wherein the fragment is at least 18 amino acids long. SEQ ID NO: 1 as depicted as follows “LRLEQVLVLVLEQFCLKVKNYSLVLSQFWLN”, refers to the so-called “decay sequence”. Such particular novel decay sequence is not mentioned at all in any down-regulation strategies on the transcriptional level as cited by the prior art (for example in Knuf et al. 2014 or in Peng et al. 2018). With regard to WO2017/004022, when aligning such degron sequence with the specific decay sequence as depicted in SEQ ID NO: 1 of the invention, no sequence identity can be found at all. Thus, the skilled person does not get any incentive or motivation to apply a completely different (not even 2% identity!)—thus not even slightly related—decay sequence which directs protein decay as the one disclosed in the present invention based on the teaching of the prior art (such as WO2017/004022). Even when combining WO2017/004022 with Knuf et al. 2014 or Peng et al. 2018, the skilled person would also not arrive at the particular decay sequence of the present invention, since none of the prior art documents discloses any similar decay sequence having at least about 60% identity to the decay sequence according to SEQ ID NO: 1 of the invention, let alone the specific one of “LRLEQVLVLVLEQFCLKVKNYSLVLSQFWLN”. The decay sequence according to SEQ ID NO: 1 of the invention is therefore not obvious at all.

### Expression Cassette

An “expression” as used in the present invention is a biological process in which the information of a DNA part is converted into a gene product, which may be an RNA molecule (gene expression) or a protein (protein expression). A gene product can be the direct transcriptional product of a gene (e.g. mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation.

The term “expression cassette” as used in the present invention means a contiguous nucleic acid molecule that can be isolated as a single unit and cloned as a single functional expression unit. A functional expression unit, capable of properly driving the expression of an incorporated polynucleotide, is thus also referred to as an “expression cassette” herein. The introduction of an expression cassette into the genome has the potential to change the phenotype of that cell by addition/deletion of a genetic sequence that permits gene expression.

For example, an expression cassette may be created enzymatically (e.g. by using type I or type II restriction endonucleases, exonucleases, etc.), by mechanical means (e.g. shearing), by chemical synthesis, or by recombinant methods (e.g. PCR). Expression cassettes generally include the following elements (presented in the 5′-3′ direction of transcription): a transcriptional and translational initiation region, a coding sequence for a gene of interest, and a transcriptional and translational termination region functional in the organism where it is desired to express the gene of interest.

The expression cassette of the invention encoding a fusion protein comprises at least two elements: a) nucleotide sequence encoding an amino acid sequence shown in SEQ ID NO: 1 or a fragment thereof which directs protein decay or a nucleotide sequence encoding an amino acid sequence which is at least 60%, at least 65%, at least 70%, at least 75%, at least 80%, at least 85%, at least 90%, at least 95% identical to the amino acid sequence as shown in SEQ ID NO: 1 or a fragment thereof which directs protein decay, and b) a nucleotide sequence encoding a protein of interest. It is preferred that the first nucleotide sequence a) is a nucleotide sequence that is different from the second nucleotide sequence b). Accordingly, the first and second nucleotide sequences are preferably heterologous to each other.

In other words, the nucleotide sequence a) comprises the coding sequence for said amino acid sequence as shown in SEQ ID NO: 1 or a fragment thereof which directs protein decay. Nucleotide sequence a) also comprises the coding sequence for an amino acid sequence which is at least 60%, at least 65%, at least 70%, at least 75%, at least 80%, at least 85%, at least 90%, at least 95% identical to SEQ ID NO: 1. The expression “coding sequence” refers to the region of continuous sequential DNA triplets encoding a protein, polypeptide or peptide sequence. Thus “encoding” describes a DNA sequence carrying information which can be transcribed and/or translated into an amino acid sequence. Preferably, the nucleotide sequence of a) is shown in SEQ ID NO: 2 which encodes an amino acid sequence shown in SEQ ID NO: 1 or a fragment thereof, encoding an amino acid sequence which is at least 60%, at least 65%, at least 70%, at least 75%, at least 80%, at least 85%, at least 90%, at least 95% identical to the amino acid sequence as shown in SEQ ID NO: 1. Such SEQ ID NO: 2 refers to the following sequence “cgcttggagcaggtgctggtgctggtgctggagcaattctgtctaaaggtgaagaattattcactggtgttgtcccaatt ttggttgaattag” which encodes the decay sequence according to SEQ ID NO: 1 as disclosed above.

“Nucleotide sequence a)” or simply “a)” is also referred to herein as “first nucleotide sequence” or, sometimes it is referred to as “element a)”. Likewise, “nucleotide sequence b)” or simply “b)” is sometimes also referred to herein as “second nucleotide sequence”.

In this context as used in the present invention, the term “nucleotide sequence” or “nucleic acid molecule” refers to a polymeric form of nucleotides (i.e. polynucleotide) of at least 10 bases in length which are usually linked from one deoxyribose or ribose to another. The term includes DNA molecules (e.g. cDNA or genomic or synthetic DNA) and RNA molecules (e.g. mRNA or synthetic RNA), as well as analogues of DNA or RNA containing non-natural nucleotide analogues, non-native internucleoside bonds, or both. The term “nucleotide sequence” does not comprise any size restrictions and also encompasses nucleotides comprising modifications, in particular modified nucleotides, e.g. as described herein. It may also refer to a specific segment of DNA, which is desired for investigation, and which may contain may include DNA regulatory elements, which control expression of the transcribed region. It can also include an intron. This gene desired for investigation may also be transcribed into RNA, may also contain an open reading frame, and may also encode a protein. In diploid organisms, a gene is composed of two alleles. An “open reading frame” describes a stretch or nucleotide region ranging from initiation codon to stop codon which is translated into protein. It is defined by the tRNA triplet system, each coding for a certain amino acid. A shift in this DNA coding triplet system or reading frame can change the resulting amino acids and thus the polypeptide chain of a protein.

Preferably, in the expression cassette of the present invention the nucleotide sequence a) is fused in frame with the nucleotide sequence b) or vice versa, i.e. the nucleotide sequence b) is fused in frame with nucleotide sequence a). Accordingly, a fusion protein is formed during translation that comprises (N-terminal) a polypeptide which directs protein decay and (C-terminal) a polypeptide of interest; or vice versa, i.e. a fusion protein comprising (N-terminal) a polypeptide of interest and (C-terminal) a polypeptide which directs protein decay.

In the context of the present invention the term “fused together” and “fused together in frame” describe that two or more nucleotide sequences as described herein, such as nucleotide sequence a) and nucleotide sequence b) as described elsewhere herein, are covalently linked together by 5′-3′ bonds of the sugar backbone of said nucleotide sequences such that these two or even more nucleotide sequences are in the same open reading frame which is then transcribed and translated as one entity. Accordingly, when the mRNA is transcribed from said covalently linked nucleic acid and translated a “fusion protein” is formed, since a ribosome translates the mRNA of these two or more nucleotide sequences as if it were one entity, i.e. the mRNA encodes one fusion protein. Said term, however, does not exclude that additional nucleotide sequences such as described elsewhere herein are contained between two nucleotide sequences such as nucleotide sequence a) and nucleotide sequence b).

However, while it is envisaged that nucleotide sequence a) and b) or b) and a) can be directly fused, i.e. meaning no additional nucleotides are between these nucleotide sequences, nucleotide sequence a) and b) or b) and a) do not have to be directly fused with each other, i.e. meaning with additional nucleotides in between.

Thus, it is also envisaged by the present invention that the expression cassette further comprises one or more (i.e. two, three, four, five, six and more) nucleotide sequence(s) (also referred to nucleotide sequence c)) which may be fused to the 5′ and/or 3′-end of the nucleotide sequence a) and/or b). In other words, one or more nucleotide sequences(s) (also referred to nucleotide sequence c)) may be fused to the 5′ and/or 3′ end of the nucleotide sequence a). It is further comprised that one or more nucleotide sequences(s) (also referred to nucleotide sequence c)) may be fused to the 5′ and/or 3′-end of the nucleotide sequence b). Additionally, it is further comprised that one or more nucleotide sequences(s) (also referred to nucleotide sequence c)) may be fused to the 5′ and 3′-end of the nucleotide sequence a) and b).

The term “5′-end” and “3′-end” are in the context of the present invention defined as features of a nucleotide sequence related to either the position of genetic elements and/or the direction of events (5′ to 3′), such as, e.g. transcription by RNA polymerase or translation by the ribosome which proceeds in 5′ to 3′ direction. Synonyms are upstream (5′) and downstream (3′). Conventionally, nucleotide sequences, gene maps, vector cards, and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left hand side, and 3′ (downstream) indicates genetic elements positioned towards the right hand side, when following this convention.

Thus, the nucleotide sequence (c) can be in between the nucleotide sequence (a) and (b) or (b) and (a). If so, the nucleotide sequence does not necessarily need to be in frame with the nucleotide sequence (a) and (b) or (b) and (a). Accordingly, nucleotide sequence (c) can be located 5′ and/or 3′ of nucleotide sequence (a) and/or (b).

However, nucleotide sequence (c) is preferably be in frame with nucleotide sequence (a) and (b) or (b) and (a). Thus, it is preferred that the nucleotide sequences (a), (b) and (c) as referred to herein, are fused in frame.

In yet a further preferred embodiment of the invention, nucleotide sequence(s) (c) is/are comprised in the nucleotide sequence (a) and/or (b). Accordingly, one or more nucleotides of the nucleotide sequence (a) and/or (b) may need to be changed so as to conform with nucleotide sequence (c).

More specifically, either the nature of the nucleotide sequence a) and/or b) is such that it comprises per se, i.e. due to its nucleotide composition one or more nucleotide sequences c) or the nucleotide sequence a) and/or b) is modified such that it then comprises one or more nucleotide sequence(s) c). For example, the codon usage can be modified by means and methods known in the art or as is described herein elsewhere. Namely, it is known that some of the naturally-occurring amino acids are encoded by one or more nucleotide triplets and this fact can be exploited when modifying nucleotide sequence a) and/or b) so as to then comprise per se one or more nucleotide sequence(s) c).

Further, said expression cassette may comprise one or more (i.e. two, three, four, five, six and more) nucleotide sequence(s) (also referred to nucleotide sequence d)) which is/are comprised in the nucleotide sequence a), b) or c). Nucleotide sequence(s) (d) is/are preferably fused in frame with the nucleotide sequence of (a), (b) and/or (c).

Thus, in a first scenario the 3′-end of nucleotide sequence a) may be fused to the 5′-end of nucleotide sequence b) encoding the protein of interest. Nucleotide sequence c) may in this scenario be combined with these nucleotide sequences a) and b), meaning that nucleotide sequence c) may be placed in-between both nucleotide sequences a) and b), at the 5′-end of nucleotide sequence a), or at the 3′-end of the nucleotide sequence b). Additionally, in this scenario nucleotide sequence d) encoding a linker, tag or cleavable site for a protease, may be placed within the nucleotide sequences a) to c) or at each end (5′- or 3′ end) of nucleotide sequence c).

Similarly, the abovementioned disclosure with regard to the nucleotide sequences c) and d) may also be applicable when the nucleotide sequence a) and b) are exchanged in a way that nucleotide sequence b) is orientated at the 5′-end and nucleotide sequence a) is orientated at the 3′-end.

In a second scenario nucleotide sequence a) may be placed in-between nucleotide sequence b) encoding the protein of interest. In this scenario the nucleotide sequence c) encoding any protein, may be placed at the 5′-end or at the 3′-end of nucleotide sequence a), or at the 5′-end or at the 3′-end of nucleotide sequence b). Also in this scenario nucleotide sequence d) encoding a linker, tag or cleavable site for a protease, may be placed within the nucleotide sequences a) to c) or at each end (5′- or 3′ end) of nucleotide sequence c).

Preferably, said nucleotide sequence d) comprises at least 3 nucleotides e.g. 3, 6, 9, 12, 15, 18, 21, 24, 27, 30 or more nucleotides. Accordingly, if nucleotide sequence (d) is fused in frame with the nucleotide sequence of (a), (b) and/or (c), said nucleotide sequence (d) encodes a heterologous polypeptide. Preferably, said heterologous polypeptide is a linker, tag and/or cleavage site for a protease.

The term “heterologous polypeptide” means herein a peptide with one or more structural or functional different units or tasks. Preferably, a heterologous polypeptide is a linker sequence, a protein tag and/or a protease recognition site enabling the cleavage of peptide.

A “linker” can be a peptide bond or a stretch of amino acids comprising at least one amino acid residue which may be arranged between the components of the fusion proteins in any order. Such a linker may in some cases be useful, for example, to improve separate folding of the individual domains or to modulate the stability of the fusion protein. Moreover, such linker residues may contain signals for transport, protease recognition sequences or signals for secondary modification. The amino acid residues forming the linker may be structured or unstructured. Preferably, the linker may be as short as 1 amino acid residue or up to 2, 3, 4, 5, 10, 20 or 50 residues. In particular cases, the linker may even involve up to 100 or 150 residues.

When used in the context of the present invention a “tag” means a protein label sequence. A tag may be used to allow identification and/or purification of the protein of interest Examples of affinity tags that may be used in accordance with the invention include, but are not limited to, HAT, FLAG, c-myc, hemagglutinin antigen, His (e.g. 6xHis) tags, flag-tag, strep-tag, strepll-tag, TAP-tag, One-Strep tag, chitin binding domain (CBD), maltose-binding protein, immunoglobulin A (IgA), His-6-tag, glutathione-S-transferase (GST) tag, intein and streptavidin binding protein (SBP) tag. It is also envisaged that said heterologous polypeptide could be a whole immunoglobulin or, preferably any Fc region of an antibody such as FcIgG, FcIgA, FcIgM, FcIgD or FcIgE.

In the context of the present invention a “cleavable site” describes an amino acid or stretch of amino acids which are recognizable by proteases. These sequences are determined by the protein structure and function of the protease. Such cleavable sites can be used to eliminate certain protein sequences when they are of no further use i.e. a protein tag or label which is intentionally enzymatically cleaved of after protein purification.

The present invention may also comprise that the expression cassette comprises a constitutive active or inducible expression control sequence which is operatively linked to the expression cassette, wherein the inducible expression control sequence is inducible preferably by temperature, light, small molecules or the expression of another protein. Thus, the expression cassette of the invention is preferably driven by an expression control sequence, i.e. its expression is controlled by an expression control sequence which is preferably either a constitutively active or inducible expression control sequence (preferably a promoter) that is operatively linked with the expression cassette.

The term “expression control sequence” as used herein refers to a polynucleotide sequence which is necessary to affect the expression of the expression cassette which it is operatively linked to. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g. ribosome binding sites); sequences that enhance protein stability; and when desired, sequences that enhance protein decay. Said expression control sequence can be constitutive active or inducible.

The term “control sequences” is intended to include, at a minimum, all components essential for expression, and can also include additional components which are advantageous, for example, leader sequences and fusion partner sequences.

As defined elsewhere herein, when the expression control sequence is preferably a promoter, such “promoter” is a nucleotide sequence which initiates and regulates transcription of a polynucleotide. It will be recognized by a person skilled in the art that any compatible promoter can be used for recombinant expression in bacterial, mammal or fungal host cells. The promoter itself may be preceded by an upstream activating sequence, an enhancer sequence or combination thereof. These sequences are known in the art as being any DNA sequence exhibiting a strong transcriptional activity in a cell and being derived from a gene encoding an extracellular or intracellular protein. It will also be recognized by a person skilled in the art that termination and polyadenylation sequences suitable may be derived from the same sources as the promoter.

An “inducible promoter” is a nucleotide sequence linking the gene expression of a further genetic element to the presence of a chemical agent, small molecule, co-factor, or regulatory protein. It is intended that the term “promoter” or “control element” includes full-length promoter regions and functional (e.g. controls transcription or translation) segments of these regions. A promoter sequence is preferably inserted upstream of the expression cassette and regulates its expression. Promoter sequences are non-coding regulatory sequences for transcription, usually located nearby the start of the gene coding sequence, which may be referred to as the gene promoter or the regulatory sequence. A constitutive active promotor is always active or switched on while an inducible promotor is only active when a certain agent or protein is bound to it.

The inducible promoter can comprise elements which are suitable for binding or interacting with the transcriptional regulator protein or other inducing elements. The interaction of the transcriptional regulator protein with the inducible promoter is preferably controlled by the exogenously supplied substance, which also refer to small molecules in the present invention. The small molecules (exogenously supplied substance) can be any suitable molecule that binds to or interacts with the transcriptional regulator protein. Suitable substances include tetracycline, ponasterone A and mifepristone.

Alternatively, inducible systems may be based on the synthetic steroid mifepristone as the small molecules as defined herein (exogenously supplied substance). In this scenario, a hybrid transcriptional regulator protein is inserted, which is based upon a DNA binding domain from the yeast GAL4 protein, a truncated ligand binding domain (LBD) from the human progesterone receptor and an activation domain (AD) from the human NF-κB. This hybrid transcriptional regulator protein is available from Thermo Fisher Scientific (Gene Switch™). Mifepristone activates the hybrid protein, and permits transcription from the inducible promoter which comprises GAL4 upstream activating sequences (UAS) and the adenovirus Elb TATA box. This system is described in Wang, Y. et al., (1994) Proc. Natl. Acad. Sci. USA 91, 8180-8184.

The induction of said expression control sequence, preferably a promoter, is also preferably achieved by the expression of another protein. Another protein may be a transcriptional regulator protein which can thus be any suitable regulator protein, either an activator or repressor protein. Suitable transcriptional activator is e.g. tetracycline-responsive transcriptional activator protein (rtTa) or the Gene Switch hybrid transcriptional regulator protein. Suitable repressor proteins include the Tet-Off version of rtTA, TetR or EcR. The transcriptional regulator proteins may be modified or derivatised as required. A transcriptional regulator protein can also mean a repressor protein, such as an ecdysone receptor or a derivative thereof. Examples include the VgEcR synthetic receptor from Agilent technologies which is a fusion of EcR, the DNA binding domain of the glucocorticoid receptor and the transcriptional activation domain of Herpes Simplex Virus VP16. The inducible promoter comprises the EcRE sequence or modified versions thereof together with a promoter. Modified versions include the E/GRE recognition sequence of Agilent Technologies, in which mutations to the sequence have been made. The E/GRE recognition sequence comprises inverted half-site recognition elements for the retinoid-X-receptor (RXR) and GR binding domains. In all permutations, the exogenously supplied substance is ponasterone A, which removes the repressive effect of EcR or derivatives thereof on the inducible promoter, and allows transcription to take place. According to the present invention, the expression control sequence (preferably a promotor) is also preferably inducible by temperature or light. Examples include but are not limited to the heat-shock-inducible Hsp70 or Hsp90-derived promotors, and the blue light sensing YFI protein, bli-3 or vvd.

As described in the context of the present invention, the term “operably linked” refers to an arrangement of genetic elements wherein the components are configured as to perform their usual function. A given promoter operably linked to a genetic sequence is capable of effecting the expression of that sequence when the proper enzymes are present. The promoter does not have to be contiguous with the sequence, as long as it functions to direct the expression thereof. For example, intervening untranslated yet transcribed sequences can be present between the promoter sequence, the genetic sequence or the promoter sequence and still be considered as “operably linked” to the genetic sequence. Thus, the term “operably linked” is intended to encompass any spacing or orientation of the promoter element and the genetic sequence in the cassette which allows for initiation of transcription of the cassette upon recognition of the promoter element by a transcription complex.

Additionally, it is also comprised by the present invention that said expression cassette may further comprise a nucleotide sequence encoding a selection marker. Preferably, said selection marker confers a resistance against an antibiotic or anti-metabolite. A “selection marker”, in accordance with the present invention, means a protein which provides the transformed cells with a selection advantage (e.g. growth advantage, resistance against an antibiotic) by expressing the corresponding gene product. Marker genes code, for example, for enzymes causing a resistance to particular antibiotics. The term “antimetabolite” as used herein refers to a substance which interferes with the normal metabolic process of a cell, typically by interacting with enzymes. This includes but is not limited to competitive inhibitors of metabolic active enzymes, substances inhibiting DNA production, or antibiotics such as sulfanilamide drugs which inhibit dihydrofolate synthesis in bacteria by competing with para-aminobenzoic acid (PABA).

### Fusion Protein

A “fusion protein” as used in the present invention which may be encoded by an expression cassette refers in general to a polypeptide comprising a first polypeptide or fragment thereof, coupled to at least another polypeptide or fragment thereof.

According to the present invention, a fusion protein comprises
i) the polypeptide having the amino acid sequence as shown in SEQ ID NO: 1 or a fragment thereof, or a polypeptide having the amino acid sequence which is at least 60% identical to SEQ ID NO: 1, which are both encoded by nucleotide sequence a) as defined above; and
ii) at least another polypeptide (protein of interest) having the amino acid sequence encoded by nucleotide sequence b) as also defined above.

Fusion proteins are useful because they can be constructed to contain two or more desired functional elements from two or more different proteins. Preferably, a fusion protein according to the present invention can be produced recombinantly by constructing a first nucleotide sequence a) which encodes a first polypeptide having an amino acid sequence as shown in SEQ ID NO: 1 or a fragment thereof, or which encodes a polypeptide having the amino acid sequence which is at least 60% identical to SEQ ID NO: 1, in-frame with at least another nucleotide sequence b) which encodes a protein of interest. Said fusion protein can also be produced recombinantly by constructing a first nucleotide sequence a) which encodes a first polypeptide having an amino acid sequence as shown in SEQ ID NO: 1 or a fragment thereof, or which encodes a polypeptide having the amino acid sequence which is at least 60% identical to SEQ ID NO: 1, in-frame with a nucleotide sequence b) which encodes a protein of interest and a third, fourth, fifth nucleotide sequence or even more nucleotide sequences encoding a further protein or peptide. According to the present invention, said fusion protein can also be produced recombinantly by constructing a nucleotide sequence a) in-frame with a nucleotide sequence b) together with one or more nucleotide sequence c) and one or more nucleotide sequence d).

Alternatively, but less preferred a fusion protein can be produced chemically by crosslinking the polypeptide or a fragment thereof to another protein.

Accordingly, said amino acid sequence as shown in SEQ ID NO: 1 or a fragment thereof, or an amino acid sequence which is at least 60%, at least 65%, at least 70%, at least 75%, at least 80%, at least 85%, at least 90%, at least 95% identical to the amino acid sequence as shown in SEQ ID NO: 1 refers to one of the polypeptides of the fusion protein, as described elsewhere herein.

The term “fragment thereof” as used herein means a fragment of an amino acid sequence of a polypeptide. A fragment in general means a polypeptide that has an amino-terminal and/or carboxyl-terminal deletion compared to a full-length polypeptide. In the context of the present invention, a fragment thereof may refer to a fragment of the amino acid sequence as shown in SEQ ID NO: 1. In a preferred embodiment, the polypeptide fragment is a contiguous sequence in which the amino acid sequence of the fragment is identical to the corresponding positions in the naturally-occurring sequence. Fragments according to the present invention are at least 5, 6, 7, 8, 9 or at least 10 amino acids long, preferably at least 12, 14, 16 or at least 18 amino acids long. The fragment as used in the present invention has the same biological activity as the full-length polypeptide or a portion thereof, namely the fragment as defined herein directs protein decay as the decay sequence of SEQ ID NO: 1 itself.

Accordingly, said protein of interest which is encoded by the nucleotide sequence b) refers to another polypeptide of the fusion protein as described elsewhere herein. Examples of a protein of interest of the present invention may be but are not limited to enzymes, receptors, receptor ligands, antibodies, lipocalins, hormones, inhibitors, membrane proteins, membrane associated proteins, peptidic toxins and peptidic antitoxins.

In this context, “peptidic toxins and peptidic antitoxins” refer to a toxin-antitoxin system in which the toxin is post-translationally bound by a protein with antitoxin function and thus inhibited. Examples for this system may be cccdB and cccdA of E. coli, or parE and parD of Caulobacter crescentus.

Thus, when the term “protein of interest” is used in the present invention, any of the proteins of interest listed above may be applicable. Preferably, the protein of interest is an enzyme, more preferably an amylolytic enzyme, a lipolytic enzyme, a proteolytic enzyme, a cellulolytic enzyme, an oxidoreductase or a plant cell-wall degrading enzyme; even more preferably an enzyme having an activity selected from the group consisting of aminopeptidase, amylase, amyloglucosidase, carbohydrase, carboxypeptidase, catalase, cellulase, chitinase, cutinase, cyclodextrin glycosyltransferase, deoxyribonuclease, esterase, galactosidase, beta-galactosidase, glucoamylase, glucose oxidase, glucosidase, haloperoxidase, hemicellulase, invertase, isomerase, laccase, ligase, lipase, lyase, mannosidase, oxidase, pectinase, peroxidase, phytase, phenoloxidase, polyphenoloxidase, protease, ribonuclease, transferase, transglutaminase, and xylanase, growth factors, cytokines, antibodies or functional fragments thereof such as Fab or F(ab)₂ or derivatives of an antibody such as bispecific antibodies (for example, scFvs), chimeric antibodies, humanized antibodies, single domain antibodies such as Nanobodies or domain antibodies (dAbs), or anticalins (lipocalin muteins).

In a most preferred embodiment of the present invention, when the protein of interest is an enzyme, it refers to a lanosterol synthase, preferably Erg7p as shown in SEQ ID NO: 3. In this context, a “lanosterol synthase” refers to an enzyme that converts 2,3-oxidosqualene to lanosterol. In a preferred embodiment the lanosterol synthase is Erg7p (E.C. 5.4.99.7).

As defined above, said fusion protein inter alia comprises a polypeptide having an amino acid sequence as shown in SEQ ID NO: 1, or having an amino acid sequence which is at least 60%, at least 65%, at least 70%, at least 75%, at least 80%, at least 85%, at least 90%, at least 95% identical to the amino acid sequence as shown in SEQ ID NO: 1, which is able to direct protein decay according to the present invention which will be described in the following.

### Protein Decay

The level of protein within a cell is determined not only by rates of synthesis, but also by rates of protein degradation. The half-lives of proteins within cells vary widely, from minutes to several days, and differential rates of protein degradation are an important aspect of cell regulation. Faulty or damaged proteins are recognized and rapidly degraded within cells, thereby eliminating the consequences of mistakes made during protein synthesis. In eukaryotic cells, two major pathways—the ubiquitin-proteasome pathway and lysosomal proteolysis—mediate protein degradation. The ubiquitin-proteasome system (UPS) for protein degradation has been under intensive study, and yet, there is only partial understanding of mechanisms by which proteins are selected to be targeted for proteolysis. One of the obstacles in studying these recognition pathways is the limited repertoire of known degradation signals. Such a degradation signal is described by the present invention as the decay sequence, which is described elsewhere herein.

The term “protein decay” as used herein refers to a protein degradation through a hydrolytic breakdown of proteins into peptides and amino acids mediated by an enzyme. In general, any proteinases or proteases capable of proteolysis may be involved in the protein degradation.

In a first step, a protein decay according to the present invention can be determined by adding a decay sequence as will be described below to a protein of interest, which then represent the fusion protein according to the present invention. This may be achieved by using the expression cassette encoding said fusion protein as described elsewhere herein, wherein said expression cassette comprises a nucleotide sequence a) encoding the decay sequence and at least another nucleotide sequence b) encoding a protein of interest and wherein said nucleotide sequences are fused together in frame.

According to the present invention, a “decay sequence” is added to said protein of interest and directs the protein decay. Such decay sequence is encoded by the nucleotide sequence a) as described elsewhere herein which is comprised by said expression cassette. Such decay sequence according to the present invention refers to said amino acid sequence shown in SEQ ID NO: 1, or a fragment thereof. Further, the preferred decay sequence which directs the protein decay can also be an amino acid sequence which is at least 60%, at least 65%, at least 70%, at least 75%, at least 80%, at least 85%, at least 90%, at least 95% identical to the amino acid sequence as shown in SEQ ID NO: 1. The decay sequence according to the present invention thus refers to the 31 amino acid sequence resulting from the frameshift in the 3′-region of ERG7 close to the wildtype stop as mentioned earlier herein; it may also be called decay-tag. The term “decay sequence” can also be used interchangeably with the amino acid sequence shown in SEQ ID NO: 1 or a fragment thereof, or with the amino acid sequence which is at least 60%, at least 65%, at least 70%, at least 75%, at least 80%, at least 85%, at least 90%, at least 95% identical to the amino acid sequence as shown in SEQ ID NO: 1.

According to the present invention, said decay sequence may be located at the N-terminus, at the C-terminus or within the protein of interest as defined elsewhere herein. It is preferred that the decay sequence according to the present invention, can be chemically altered and is thus accessible for any enzyme involved in protein degradation on the surface of the protein of interest. In other words, a decay sequence which is added to the protein of interest, but not accessible for an enzyme involved in protein degradation such as an ubiquitinase as defined above or any proteinase or proteases capable of proteolysis, is not beneficial for the present invention.

The decay sequence of the present invention as described elsewhere herein is a completely synthetic, non-coding sequence of 31 amino acids which refers to SEQ ID NO: 1. Also encompassed by the present invention and described elsewhere herein, is a decay sequence which has at least 60%, at least 65%, at least 70%, at least 75%, at least 80%, at least 85%, at least 90%, at least 95% sequence identity to the synthetic decay sequence of SEQ ID NO: 1. This sequence identity encompasses amino acid substitutions, and especially conservative amino acid substitutions as further described below.

“Percent (%) amino acid sequence identity” with respect to amino acid sequences disclosed herein is defined as the percentage of amino acid residues in a candidate sequence that is identical with the amino acid residues in a reference sequence. After aligning the sequences and introducing gaps, if necessary, to achieve the maximum-percent sequence identity, and not considering any conservative substitutions as part of the sequence identity. Alignment for purposes of determining percent amino acid sequence identity can be achieved in various ways that are known in the skill of the art, for instance, using publically available computer software such as BLAST, ALIGN, or Megalign (DNASTAR) software. Those skilled in the art can determine appropriate parameters for measuring alignment, including any algorithms needed to achieve maximum alignment over the full length of the sequences being compared. The same is true for nucleotide sequences disclosed herein.

In general, a conservative amino acid substitution will not substantially change the functional properties of a protein. In cases where two or more amino acid sequences differ from each other by conservative substitutions, the percent-sequence-identity or degree of homology may be adjusted upwards to correct for the conservative nature of the substitution. Means for making this adjustment are well known to the skilled artisan.

Examples for conservative amino acid substitutions are described in the following. These six groups contain amino acids that are conservative substitutions for one another: 1) Serine(S), Threonine (T); 2) Aspartic Acid (D), Glutamic Acid (E); 3) Asparagine (N), Glutamine (Q); 4) Arginine (R), Lysine (K); 5) Isoleucine (1), Leucine (L), Methionine (M), Alanine (A), Valine (V), and 6) Phenylalanine (F), Tyrosine (Y), Tryptophan (W).

Sequence homology of polypeptides, also referred to as percent-sequence-identity, is typically measured by using sequence analysis software. See, e.g. the Sequence Analysis Software Package of the Genetics Computer Group (GCG), University of Wisconsin Biotechnology Center, 910 University Avenue, Madison, Wisconsin 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e. g. GCG Version 6.1.

A preferred algorithm when comparing a molecule sequence to a database containing a large number of sequences from different organisms is the computer program BLAST (Altschul et al., (1990) J Mol. Biol. 215:403-410), especially blastp or tblastn (Altschul et al., 1997).

After having added said nucleotide sequence encoding said decay sequence in a first step as defined elsewhere herein to the nucleotide sequence encoding the protein of interest as also defined elsewhere herein, the amount/level of said fusion protein is measured in a second step after translation of the protein. When a decay sequence is added to a protein of interest in a host cell, this leads to a protein with a reduced half-life, further resulting in a lower protein level of the protein of interest in comparison to a wildtype host cell. Thus, a lower amount/level of the fusion protein comprising the protein of interest and the decay sequence results in a lower activity of said protein of interest in the mutant host cell (e.g. Simo 1575-gt-ERG7, Simo 1575-m-ERG7, Simo 1575-t-ERG7, Simo1575-o-ERG7) compared to a reference host cell which may be demonstrated as indicated by a high triterpenoid production and a reduced sterol synthesis (see Example section). In another scenario, the protein level can be measured in a host cell before and after the nucleotide sequence encoding said decay sequence is genetically added to the nucleotide sequence of said protein of interest in the host cell (meaning over a course of time), resulting in a reduced protein level after the nucleotide sequence encoding said decay sequence has been added.

By “gradual reduction” of said level of the fusion protein (or any grammatical form thereof such as “to gradually reduce”) according to the present invention is meant in general a 99%, 90%, 80%, 70%, 60%, 50%, 40%, 30%, 20%, 10% or a less than 10% reduction of said level of the fusion protein when expressed in a cell in comparison to a cell which expresses the protein of interest without said decay sequence, as described elsewhere herein. It may also be comprised herein that by said “gradual reduction” a reduction (as defined above with regard to the %-disclosure) of said level of the fusion protein over a course of time when expressed in a cell in comparison to a cell which expresses the protein of interest without said decay sequence as described elsewhere herein is meant. Thus, it may also be comprised herein that said amount/level of the fusion protein is measured by the gradual reduction over a course of time when expressed in a cell in comparison to a cell which expresses the protein of interest. The level of the fusion protein can be measured as total yield by absorbance, or after fusing the fusion protein of the present invention to a fluorescent label by determining the decrease or lesser amount of emitted light.

The term “over a course of time” refers to a period of time which may be two days, one day, 18 hours, 12 hours, 6 hours, 5 hours, 4 hours, 3 hours, 2 hours, 1 hour or less than 1 hour. A decreasing amount/level of said fusion protein when measuring said amount/level over a course of time represents a decay of said fusion protein according to the present invention. Thus, when adding a decay sequence to a protein of interest, it results in a gradual reduction of the level of fusion protein over a course of time in comparison to the natural protein expression of the protein of interest, which does not have an added decay sequence. In other words, a protein decay according to the present invention may also refer to a gradual reduction of the level of the fusion protein over a course of time as defined elsewhere herein in comparison to a cell which expresses the protein of interest without a decay sequence. A “course of time” as used in the present invention may be defined as at least the natural protein half-life of the native protein of interest. For example, when the protein of interest is Erg7p as defined elsewhere herein, the protein half-life of said protein is at least several hours (e.g. about 12 h).

When the protein of interest of the fusion protein is an enzyme which is preferred in the present invention, the gradual reduction of said enzymatic activity of the fusion protein can be measured. Such gradual reduction of said enzymatic activity refers to a reduction of 99%, 90%, 80%, 70%, 60%, 50%, 40%, 30%, 20%, 10% or less than 10% in comparison to said enzymatic activity of an enzyme without the decay sequence as described elsewhere herein. The enzymatic activity of the fusion protein can be determined dependent on the analysed enzyme. General approaches include fusing the substrate of the enzyme to a quenched fluorescent label and measuring the emitted light of the cleaved reaction product. Such measurements may include besides fluorometric methods, calorimetric measurements, chemoluminescent light emissions, microscale thermophoresis, light scattering, and radiometric or chromatographic assays.

Alternatively, when the protein of interest of the fusion protein is an enzyme which is preferred in the present invention, the gradual reduction of the product yield of the enzymatic reaction can be measured. Such gradual reduction of the product yield of the enzymatic reaction refers to a reduction of 99%, 90%, 80%, 70%, 60%, 50%, 40%, 30%, 20%, 10% or less than 10% in comparison to the product yield of a cell which expresses the enzyme without the decay sequence as described elsewhere herein. Also in this scenario the nature of the product determines the way, how the product can be analysed and are known to those skilled in the art. This means, that if the enzyme produces a cytotoxic product, the attachment of the decay sequence as described elsewhere herein to the enzyme as defined herein may lead to a growth advantage of the cell resulting in increasing cell viability with efficient protein decay due to the fact that there is a gradual reduction of the cytotoxic product yield of the enzymatic reaction. The increasing cell viability thus represents the protein decay.

As mentioned above in a first step, a protein decay according to the present invention can be determined by adding a decay sequence as will be described below to a protein of interest, which then represent the fusion protein according to the present invention. The second step of the determination of protein decay which comprises a measuring step as defined above can be further accompanied by one or more fluorescent label(s) linked to said protein of interest as described elsewhere herein. Such a fluorescent label can be linked to any position of the protein of interest and visualizes its deposition. It may be advantageous to combine two fluorescent labels, emitting light of different wavelength, in a way that one label is linked to the decay sequence and another is linked to the protein of interest.

In sum, the protein decay is analysed by the functional test of adding the decay sequence as defined elsewhere herein to the protein of interest and then measuring the level of said obtained fusion protein as defined elsewhere herein (e.g. measuring over a course of time). A reduction or gradual reduction of the level of said fusion protein compared to the reference strain as defined elsewhere herein represents a protein decay according to the present invention.

### Vector

Also encompassed by the present invention is that the expression cassette as defined elsewhere herein is comprised by a vector. As used within the present invention, the term “vector” is a nucleic acid molecule, such as a DNA molecule, which is used as a vehicle to artificially carry genetic material into a cell. The vector is generally a nucleic acid sequence that consists of an insert (such as a nucleic acid sequence or gene) and a larger sequence that serves as the “backbone” of the vector. The vector may be in any suitable format, including plasmids, mini-circle, or linear DNA. The vector may comprise at least the nucleic acid sequence a) and the sequence of a protein of interest. Optionally, the vectors also possess an origin of replication (ori), which permits amplification of the vector, for example in bacteria. Additionally, or alternatively, the vector may include selectable markers such as antibiotic resistance genes, genes for coloured markers and suicide genes. Preferably, this selection marker is capable of being incorporated in the genome of the host organism upon transformation, and was not expressed functionally by the host prior to transformation. Transformed host cells can then be selected and isolated from untransformed cells on the basis of the incorporated selection marker. In particular, the nucleotide sequence serving as the selectable marker genes as well as the nucleotide sequence encoding the protein of interest can be transcribed under the control of transcription elements present in appropriate promoters. The resulting transcripts of the selectable marker genes and the protein of interest harbour functional translation elements that facilitate substantial levels of protein expression (i.e. translation) and proper translation termination. The vector can contain one or more unique restriction sites for this purpose and may be capable of autonomous replication in a bacterial, mammalian or fungal host cell or may be ectopically or homologously integrated. Also, the vector may comprise a polylinker (multiple cloning site), i.e. a short segment of DNA that contains many restriction sites, a standard feature on many plasmids used for molecular cloning. Multiple cloning sites typically contain more than 5, 10, 15, 20, 25, or more than 25 restrictions sites. Restriction sites within an MCS are typically unique (i.e. they occur only once within that particular plasmid). MCSs are commonly used during procedures involving molecular cloning or subcloning.

One type of vector is a “plasmid”, which refers to a circular double stranded DNA loop into which additional DNA segments may be ligated. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g. vectors having an origin of replication which functions in the host cell). Other vectors can be integrated into the genome of a fungal host cell upon introduction into the host cell, and are thereby replicated along with the host genome. Moreover, certain preferred vectors are capable of directing the expression of the expression cassette to which they are operatively linked. Such vectors are referred to herein as “recombinant expression vectors” (or simply, “expression vectors”).

The expression cassette is inserted into the expression vector as a DNA construct. This DNA construct can be recombinantly made from a synthetic DNA molecule, a genomic DNA molecule, a cDNA molecule or a combination thereof. In one scenario the gene coding for the protein of interest may be part of the expression vector. The vector conveniently comprises sequences that facilitate the proper expression of the expression cassette of the invention. These sequences typically comprise promoter sequences, transcription initiation sites, transcription termination sites, and polyadenylation functions as described herein. For proper expression of the polypeptides, suitable translational control elements are preferably included in the vector, such as, e.g. 5′ untranslated regions leading to 5′ cap structures suitable for recruiting ribosomes and stop codons to terminate the translation process.

TABLE 1
Listing of amino acid (Aac) and nucleotide (Nuc) sequences of the present invention

| | |
|---|---|
| SEQ ID | Decay_Aac |
| NO: 1 | “LRLEQVLVLVLEQFCLKVKNYSLVLSQFWLN” |
| SEQ ID | Decay_Nuc |
| NO: 2 | “cgcttggagcaggtgctggtgctggtgctggagcaattctgtctaaaggtgaagaattattcactggtgttgtcccaatt |
| | ttggttgaattag” |
| SEQ ID | ERG7 native_Aac |
| NO: 3 | “MTEFYSDTIGLPKTDPRLWRLRTDELGRESWEYLTPQQAANDPPSTFTQWLLQDP |
| | KFPQPHERNKHSPDFSAFDACHNGASFFKLLQEPDSGIFPCQYKGPMFMTIGYVAV |
| | NYIAGIEIPEHERIELIRYIVNTAHPVDGGWGLHSVDKSTVFGTVLNYVILRLLGLPKD |
| | HPVCAKARSTLLRLGGAIGSPHWGKIWLSALNLYKWEGVNPAPPETWLLPYSLPMH |
| | PGRWWVHTRGVYIPVSYLSLVKFSCPMTPLLEELRNEIYTKPFDKINFSKNRNTVCG |
| | VDLYYPHSTTLNIANSLVVFYEKYLRNRFIYSLSKKKVYDLIKTELQNTDSLCIAPVNQ |
| | AFCALVTLIEEGVDSEAFQRLQYRFKDALFHGPQGMTIMGTNGVQTWDCAFAIQYF |
| | FVAGLAERPEFYNTIVSAYKFLCHAQFDTECVPGSYRDKRKGAWGFSTKTQGYTVA |
| | DCTAEAIKAIIMVKNSPVFSEVHHMISSERLFEGIDVLLNLQNIGSFEYGSFATYEKIKA |
| | PLAMETLNPAEVFGNIMVEYPYVECTDSSVLGLTYFHKYFDYRKEEIRTRIRIAIEFIKK |
| | SQLPDGSWYGSWGICFTYAGMFALEALHTVGETYENSSTVRKGCDFLVSKQMKDG |
| | GWGESMKSSELHSYVDSEKSLVVQTAWALIALLFAEYPNKEVIDRGIDLLKNRQEES |
| | GEWKFESVEGVFNHSCAIEYPSYRFLFPIKALGMYSRAYETHTL” |
| SEQ ID | ERG7 native_Nuc |
| NO: 4 | “atgacagaattttattctgacacaatcggtctaccaaagacagatccacgtctttggagactgagaactgatgagcta |
| | ggccgagaaagctgggaatatttaacccctcagcaagccgcaaacgacccaccatccactttcacgcagtggcttct |
| | tcaagatcccaaatttcctcaacctcatccagaaagaaataagcattcaccagatttttcagccttcgatgcgtgtcataa |
| | tggtgcatcttttttcaaactgcttcaagagcctgactcaggtatttttccgtgtcaatataaaggacccatgttcatgacaa |
| | tcggttacgtagccgtaaactatatcgccggtattgaaattcctgagcatgagagaatagaattaattagatacatcgtca |
| | atacagcacatccggttgatggtggctggggtctacattctgttgacaaatccaccgtgtttggtacagtattgaactatgt |
| | aatcttacgtttattgggtctacccaaggaccacccggtttgcgccaaggcaagaagcacattgttaaggttaggcggt |
| | gctattggatcccctcactggggaaaaatttggctaagtgcactaaacttgtataaatgggaaggtgtgaaccctgccc |
| | ctcctgaaacttggttacttccatattcactgcccatgcatccggggagatggtgggttcatactagaggtgtttacattcc |
| | ggtcagttacctgtcattggtcaaattttcttgcccaatgactcctcttcttgaagaactgaggaatgaaatttacactaaac |
| | cgtttgacaagattaacttctccaagaacaggaataccgtatgtggagtagacctatattacccccattctactactttga |
| | atattgcgaacagccttgtagtattttacgaaaaatacctaagaaaccggttcatttactctctatccaagaagaaggttt |
| | atgatctaatcaaaacggagttacagaatactgattccttgtgtatagcacctgttaaccaggcgttttgcgcacttgtcac |
| | tcttattgaagaaggggtagactcggaagcgttccagcgtctccaatataggttcaaggatgcattgttccatggtccac |
| | agggtatgaccattatgggaacaaatggtgtgcaaacctgggattgtgcgtttgccattcaatactttttcgtcgcaggcc |
| | tcgcagaaagacctgaattctataacacaattgtctctgcctataaattcttgtgtcatgctcaatttg acaccgagtgcgt |
| | tccaggtagttatagggataagagaaagggggcttggggcttctcaacaaaaacacagggctatacagtggcagatt |
| | gcactgcagaagcaattaaagccatcatcatggtgaaaaactctcccgtctttagtgaagtacaccatatgattagcag |
| | tgaacgtttatttgaaggcattgatgtgttattgaacctacaaaacatcggatcttttgaatatggttcctttgcaacctatg |
| | aaaaaatcaaggccccactagcaatggaaaccttgaatcctgctgaagtttttggtaacataatggtagaatacccatac |
| | gtggaatgtactgattcatccgttctggggttgacatattttcacaagtacttcgactataggaaagaggaaatacgtaca |
| | cgcatcagaatcgccatcgaattcataaaaaaatctcaattaccagatggaagttggtatggaagctggggtatttgtttt |
| | acatatgccggtatgtttgcattggaggcattacacaccgtgggggagacctatgagaattcctcaacggtaagaaaa |
| | ggttgcgacttcttggtcagtaaacagatgaaggatggcggttggggggaatcaatgaagtccagtgaattacatagtt |
| | atgtggatagtgaaaaatcgctagtcgttcaaaccgcatgggcgctaattgcacttcttttcgctgaatatcctaataaag |
| | aagtcatcgaccgcggtattgaccttttaaaaaatagacaagaagaatccggggaatggaaatttgaaagtgtagaa |
| | ggtgttttcaaccactcttgtgcaattgaatacccaagttatcgattcttattccctattaaggcattaggtatgtacagca |
| | gggcatatgaaacacatacgctttaa” |
| SEQ ID | tHMG-COA_Aac |
| NO: 5 | |
| SEQ ID | OWE_Aac |
| NO: 6 | |
| SEQ ID | CYP16A12_Aac |
| NO: 7 | |
| SEQ ID | CYP716A15_Aac |
| NO: 8 | |
| SEQ ID | CYP16A17_Aac |
| NO: 9 | |
| SEQ ID | CYP16A47_Aac |
| NO: 10 | |
| SEQ ID | ATR1_Aac |
| NO: 11 | |
| SEQ ID | ATR2_Aac |
| NO: 12 | |
| SEQ ID | LjCPR1_Aac |
| NO: 13 | |
| SEQ ID | CrCP_Aac |
| NO: 14 | |
| SEQ ID | ARE1_Aac |
| NO: 15 | “MTETKDLLQDEEFLKIRRLNSAEPNKRHSVTYDNVILPQESMEVSPRSSTTSLVEPV |
| | ESSEAERVAGKQEQEEEYPVDAHMQKYLSHLKSKSRSRFHRKDASKYVSFFGDVS |
| | FDPRPTLLDSAINVPFQTTFKGPVLEKQLKNLQLTKTKAKATVKATVKTTEKTDKADA |
| | PPGEKLESNFSGIYVFAWMFLGWIAIRCCTDYYASYGSAWNKLEIVQYMTTDLFTIA |
| | MLDLAMFLCTFFVVFVHWLVKKRIINWKWTGFVAVSIFELAFIPVTFPIYVYYFDFNW |
| | VTRIFLFLHSVVFVMKSHSFAFYNGYLWDIKQELEYSSKQLQKYKESLSPETREILQK |
| | SCDFCLFELNYQTKDNDFPNNISCSNFFMFCLFPVLVYQINYPRTSRIRWRYVLEKV |
| | CAIIGTIFLMMVTAQFFMHPVAMRCIQFHNTPTFGGWIPATQEWFHLLFDMIPGFTVL |
| | YMLTFYMIWDALLNCVAELTRFADRYFYGDWWNCVSFEEFSRIWNVPVHKFLLRHV |
| | YHSSMGALHLSKSQATLFTFFLSAVFHEMAMFAIFRRVRGYLFMFQLSQFVWTALS |
| | NTKFLRARPQLSNVVFSFGVCSGPSIIMTLYLTL” |
| SEQ ID | ARE2_Aac |
| NO: 16 | “MDKKKDLLENEQFLRIQKLNAADAGKRQSITVDDEGELYGLDTSGNSPANEHTATTI |
| | TQNHSVVASNGDVAFIPGTATEGNTEIVTEEVIETDDNMFKTHVKTLSSKEKARYRQ |
| | GSSNFISYFDDMSFEHRPSILDGSVNEPFKTKFVGPTLEKEIRRREKELMAMRKNLH |
| | HRKSSPDAVDSVGKNDGAAPTTVPTAATSETVVTVETTIISSNFSGLYVAFWMAIAFG |
| | AVKALIDYYYQHNGSFKDSEILKFMTTNLFTVASVDLLMYLSTYFVVGIQYLCKWGVL |
| | KWGTTGWIFTSIYEFLFVIFYMYLTENILKLHWLSKIFLFLHSLVLLMKMHSFAFYNGY |
| | LWGIKEELQFSKSALAKYKDSINDPKVIGALEKSCEFCSFELSSQSLSDQTQKFPNNI |
| | SAKSFFWFTMFPTLIYQIEYPRTKEIRWSYVLEKICAIFGTIFLMMIDAQILMYPVAMR |
| | ALAVRNSEWTGILDRLLKWVGLLVDIVPGFIVMYILDFYLIWDAILNCVAELTRFGDRY |
| | FYGDWWNCVSWADFSRIWNIPVHKFLLRHVYHSSMSSFKLNKSQATLMTFFLSSVV |
| | HELAMYVIFKKLRFYLFFFQMLQMPLVALTNTKFMRNRTIIGNVIFWLGICMGPSVMC |
| | TLYLTF” |
| SEQ ID | AaBas_Aac |
| NO: 17 | |
| SEQ ID | PgDDS_Aac |
| NO: 18 | |
| SEQ ID | Simo1575-t-ERG7_Aac |
| NO: 19 | |
| SEQ ID | Simo1575-m-ERG7_Aac |
| NO: 20 | |
| SEQ ID | tHMG-COA_Nuc |
| NO: 21 | |
| SEQ ID | OWE_Nuc |
| NO: 22 | |
| SEQ ID | CYP16A12_Nuc |
| NO: 23 | |
| SEQ ID | CYP716A15_Nuc |
| NO: 24 | |
| SEQ ID | CYP16A17_Nuc |
| NO: 25 | |
| SEQ ID | CYP16A47_Nuc |
| NO: 26 | |
| SEQ ID | ATR1_Nuc |
| NO: 27 | |
| SEQ ID | ATR2_Nuc |
| NO: 28 | |
| SEQ ID | LjCPR1_Nuc |
| NO: 29 | |
| SEQ ID | CrCP_Nuc |
| NO: 30 | |
| SEQ ID | ARE1_Nuc |
| NO: 31 | |
| SEQ ID | ARE2_Nuc |
| NO: 32 | |
| SEQ ID | AaBas_Nuc |
| NO: 33 | |
| SEQ ID | PgDDS_Nuc |
| NO: 34 | |
| SEQ ID | Simo1575-t-ERG7_Nuc |
| NO: 35 | |
| SEQ ID | Simo1575-m-ERG7_Nuc |
| NO: 36 | |
| SEQ ID | Simo1575-gt-ERG7_Aac |
| NO: 37 | |
| SEQ ID | Simo1575-gt-ERG7_Nuc |
| NO: 38 | |
| SEQ ID | Simo1575-o-ERG7_Aac |
| NO: 39 | |
| SEQ ID | Simo1575-0-ERG7_Nuc |
| NO: 40 | |
| SEQ ID | PERG7 (500 bp upstream start codon) + ERG7mut + disrupted |
| NO: 41 | degron + PTEF1 + hygromycin resistance_Nuc |
| SEQ ID | frameshift ERG7 decay mutant + disrupted degron_Aac |
| NO: 42 | |
| SEQ ID | frameshift ERG7 decay mutant + disrupted degron_Nuc |
| NO: 43 | |
| SEQ ID | PERG7 + frameshift decay mutant ERG7mut + disrupted degron till pos 2331 + |
| NO: 44 | PTEF1 + bleR |
| SEQ ID | frameshift ERG7 mutant (native ERG7 till pos 2185 + sequence downstream of the |
| NO: 45 | frameshift mutation till new stop codon)_Aac |
| SEQ ID | frameshift ERG7 mutant (native ERG7 till pos 2185 + sequence downstream of the |
| NO: 46 | frameshift mutation till new stop codon)_Nuc |

### Host Cell

In a further embodiment either the expression cassette as described elsewhere herein or the vector comprising the expression cassette according to the present invention is comprised in a host cell. The term “host cell” as used herein, is intended to refer to a cell into which a nucleotide sequence comprising said expression cassette or said vector comprising the expression cassette as described herein, has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

Said host cell which comprises the expression vector as defined herein may comprise a lanosterol synthase, preferably Erg7p as shown in SEQ ID NO: 3, as the protein of interest. Preferably, said lanosterol synthase comprised by the expression cassette is encoded by the nucleotide sequence as shown in SEQ ID NO: 4.

Said host cell described herein may be of bacterial, mammalian or fungal origin. Said host cell may be an isolated host cell, which can be grown in culture. In a preferred embodiment said host cell is a fungal host cell. Preferably, the fungal host cell is capable of growth in liquid medium. Preferably, the fungal host cell is a filamentous fungus belonging to the genus of Aspergillus, e.g. A. niger, A. awamori, A. oryzae, A. nidulans, a yeast belonging to the genus of Saccharomyces, e.g. S. cerevisiae, S. sensu stricto, S. kluyveri, S. bayanus, S. exiguus, S. sevazzi, S. uvarum, a yeast belonging to the genus Kluyveromyces, e.g. K. lactis K. marxianus var. marxianus, K. thermotolerans, a yeast belonging to the genus Candida, e.g. C. utilis C. tropicalis, C. albicans, C. lipolytica, C. versatilis, a yeast belonging to the genus Pichia, e.g. P. stipidis, P. pastoris, P. sorbitophila, or other yeast genera, e.g. Cryptococcus, Debaromyces, Hansenula, Pichia, Yarrowia, Zygosaccharomyces or Schizosaccharomyces. Concerning other microorganisms a non-exhaustive list of further suitable filamentous fungi is supplied which may be used herein: a species belonging to the genus Penicillium, Rhizopus, Fusarium, Fusidium, Gibberella, Mucor, Mortierella, Trichoderma. In an even more preferred embodiment, said host cell is a yeast host cell. In a most preferred embodiment, said host cell is a basidiomycetous or hemiascomycetous yeast host cell. A particularly preferred host cell is Saccharomyces cerevisiae, Saccharomyces sensu stricto, and Ustilago maydis. Therefore, the present invention relates to a (recombinant) fungal host cell comprising the expression cassette or the vector described herein.

The host cell as described herein may further not express one or more sterol acyltransferases, preferably: (i) Are1p as shown in SEQ ID NO: 15 and/or (ii) Are2p as shown in SEQ ID NO: 16, due to genetic deletion of ARE1 and/or ARE2. When the sterol acyltransferase refers to the enzyme as shown in SEQ ID NO: 15, said enzyme is encoded by the nucleotide sequence as shown in SEQ ID NO: 31. When the sterol acyltransferase refers to the enzyme as shown in SEQ ID NO: 16, said enzyme is encoded by the nucleotide sequence as shown in SEQ ID NO: 32. In combination with a protein decay of preferably Erg7p or another enzyme of the mevalonate pathway as defined elsewhere herein, such a deletion of ARE1 and/or ARE2 (see e.g. Simo1575 or BA6 strain having an ARE2 deletion) results in improved production titers of triterpenoids. Homologue proteins of Are1p and Are2p expressed in other yeast strains are also encompassed within the present invention.

Additionally or alternatively, the present invention also describes a host cell expressing one or more of the following proteins: (i) a truncated HMG-COA reductase; (ii) an oxidosqualene cyclase; (iii) a cytochrome P450 monooxygenase; (iv) a cytochrome P450 reductase; (v) a sterol acyltransferase.

Expression of the truncated HMG-COA reductase (short: tHMG-COA reductase) results in an increased mevalonate pathway flux, and the synthesis of farnesyl pyrophosphate (FPP). Squalene synthesis follows: Squalene synthase (Erg9p) catalyses the reductive dimerization of FPP, in which two molecules of FPP are converted into one molecule of squalene. Two molecules of this metabolite are then reductively. Squalene is then oxygenated by the squalene monooxygenase (Erg1p) to 2,3-oxidosqualene, which in a natural yeast strain is transformed into the sterol pathway, starting with the lanosterol synthase (Erg7p) mediated conversion to lanosterol and ending with the formation of the final product ergosterol. In the engineered strain, expressing a heterologous oxidosqualene cyclase (OSC), 2,3-oxidosqualene is cyclised to a broad variety of triterpenoids, e.g. the pentacyclic molecules, lupeol and β-amyrin or the tetracyclic dammarenediol. These cyclic products are further functionalised by P4500 monooxygenases, introducing oxygen atoms at diverse positions, e.g. lupeol can be converted to betulinic acid in a three step oxidation catalysed by the P450 monooxygenase CYP716A15 with the oxidation of NADPH to NADP+ (see FIG. 1). Catalytic enzymes of the above described pathway, such as Erg1p, Erg9p, or Erg20p may be also be defined as the protein of interest according to the present invention. Therefore, Erg1p may be combined with the decay sequence shown in SEQ ID NO: 1, Erg9p may be combined with the decay sequence shown in SEQ ID NO: 1, or Erg20p may be combined with the decay sequence shown in SEQ ID NO: 1.

When used in the present invention a “truncated HMG-COA reductase or tHMG-COA reductase” includes, but is not limited to a 3-hydroxy-3-methylglutaryl-coenzyme A reductase as shown in SEQ ID NO: 5. Said tHMG-COA reductase as shown in SEQ ID NO: 5 may be encoded by the nucleotide sequence as shown in SEQ ID NO: 21. This cytosolic enzyme catalyses the reduction of reducing (S)-3-hydroxy-3-methylglutaryl-CoA (HMG-COA) to mevalonate.

When the term “oxidosqualene cyclase (OSC)” is used herein, it includes, but is not limited to lupeol synthase, beta-amyrin synthase and dammarenediol synthase. Thus, said OSC may refer to a heterologous OSC. Other oxidosqualene cyclases (OSC) may also be comprised in the present invention, as shown by the following table 2:

| Name | Type | Plant | Description | Genbank |
|---|---|---|---|---|
| AaBAS | OSC | Artemisia annua | Artemisia annua beta-amyrin synthase | EU330197 |
| | | | (BAS) mRNA, complete cds. | |
| AaCAS | OSC | Artemisia annua | Artemisia annua cycloartenol synthase | KM670093 |
| | | | mRNA, complete cds. | |
| AaLUS | OSC | Artemisia annua | Artemisia annua lupeol synthase | KM670094 |
| | | | mRNA, complete cds. | |
| AaOSC2 | OSC | Artemisia annua | Multi-functional OSC | KF309252 |
| AaOSC3 | OSC | Artemisia annua | unknown | KM670095 |
| AcACX | OSC | Adiantum | Adiantum capillus-veneris ACX mRNA | AB368375 |
| | | capillus-veneris | for cycloartenol synthase, complete cds. | |
| AsbAS1 | OSC | Avena strigosa | Avena strigosa mRNA for beta-amyrin | AJ311789 |
| | | | synthase (bAS1 gene) | |
| AsCS1 | OSC | Avena strigosa | Avena strigosa mRNA for cycloartenol | AJ311790 |
| | | | synthase (cs1 gene) | |
| AsOXA1 | OSC | Aster sedifolius | Aster sedifolius beta-amyrin synthase | AY836006 |
| | | | (OXA1) mRNA, complete cds. | |
| AsSAD1 (S728F) | OSC | Avena strigosa | dammaranediol-II synthase | |
| AtaSHS | OSC | Aster tataricus | Aster tataricus SHS1 mRNA for | AB609123 |
| | | | shionone synthase, complete cds. | |
| AtBARS1 | OSC | Arabidopsis thaliana | Arabidopsis thaliana baruol synthase 1 | NM_117625 |
| | | | (BARS1), partial mRNA. | |
| AtBAS | OSC | Arabidopsis thaliana | Arabidopsis thaliana AtBAS mRNA | AB374428 |
| | | | for beta-amyrin synthase, complete cds. | |
| AtCAMS1 | OSC | Arabidopsis thaliana | Arabidopsis thaliana camelliol C | NM_148667 |
| | | | synthase 1 (CAMS1), partial mRNA. | |
| AtCAS | OSC | Arabidopsis thaliana | Arabidopsis thaliana cycloartenol | NM_126681 |
| | | | synthase 1 (CAS1), mRNA. | |
| AtLAS1 | OSC | Arabidopsis thaliana | Arabidopsis thaliana lanosterol | NM_114382 |
| | | | synthase 1 (LAS1), mRNA. | |
| AtLUP1 | OSC | Arabidopsis thaliana | Arabidopsis thaliana lupeol synthase | U49919 |
| | | | mRNA, complete cds. | |
| AtLUP2 | OSC | Arabidopsis thaliana | Multi-functional OSC | NM_106545 |
| AtLUP5 | OSC | Arabidopsis thaliana | Multi-functional OSC | NM_105367 |
| AtMRN1 | OSC | Arabidopsis thaliana | Arabidopsis thaliana marneral synthase | NM_123624 |
| | | | (MRN1), mRNA. | |
| AtPEN1 | OSC | Arabidopsis thaliana | Arabidopsis thaliana pentacyclic | NM_117622 |
| | | | triterpene synthase 1 (PEN1), mRNA. | |
| AtPEN3 | OSC | Arabidopsis thaliana | Pentacyclic triterpene synthase | NM_123006 |
| AtPEN6 | OSC | Arabidopsis thaliana | Multi-functional OSC | NM_106497 |
| AtTHAS1 | OSC | Arabidopsis thaliana | Arabidopsis thaliana thalianol synthase | NM_124175 |
| | | | 1 (THAS1), mRNA. | |
| BfOSC1 | OSC | Bauhinia fortificata | multi-functional OSC | |
| BfOSC2 | OSC | Bauhinia fortificata | multi-functional OSC | |
| BfOSC3 | OSC | Bauhinia fortificata | OSC | |
| BfOSC4 | OSC | Bauhinia fortificata | OSC | |
| BgBAS | OSC | Bruguiera gymnorhiza | Bruguiera gymnorhiza BgbAS mRNA | AB289585 |
| | | | for beta amyrin synthase, complete cds. | |
| BgLUS | OSC | Bruguiera gymnorhiza | Bruguiera gymnorhiza BgLUS mRNA | AB289586 |
| | | | for lupeol synthase, complete cds. | |
| BpCASBPX1 | OSC | Betula platyphylla | Betula platyphylla CASBPX1 mRNA | AB055509 |
| | | | for cycloartenol synthase, complete cds. | |
| BpCASBPX2 | OSC | Betula platyphylla | Betula platyphylla CASBPX2 mRNA | AB055510 |
| | | | for cycloartenol synthase, complete cds. | |
| BpOSCBPW | OSC | Betula platyphylla | Betula platyphylla OSCBPW mRNA | AB055511 |
| | | | for lupeol synthase, complete cds. | |
| BpOSCBPY | OSC | Betula platyphylla | Betula platyphylla OSCBPY mRNA | AB055512 |
| | | | for beta-amyrin synthase, complete cds. | |
| BvLUP2G | OSC | Barbarea vulgaris | lupeol synthase | KP784688 |
| BvLUP2P | OSC | Barbarea vulgaris | lupeol synthase | KP784687 |
| BvLUP5G | OSC | Barbarea vulgaris | multi-functional OSC | KP784690 |
| BvLUP5P | OSC | Barbarea vulgaris | multi-functional OSC | KP784689 |
| CaCYS | OSC | Centella asiatica | Centella asiatica cycloartenol synthase | AY520819 |
| | | | (OSCCCS) mRNA, complete cds. | |
| CaDDS | OSC | Centella asiatica | multi-functional OSC | AY520818 |
| CbβAS | OSC | Conyza blinii | β-amyrin synthase | KJ650043 |
| CcCDS1 | OSC | Citrullus colocynthis | lanosterol synthase | KM821404 |
| CcCDS2 | OSC | Citrullus colocynthis | multi-functional OSC | KM821405 |
| ClBi | OSC | Citrullus lanatus | cucurbitadienol synthase | KM821403 |
| CmBi/CmCPQ | OSC | cucumis melo | cucurbitadienol synthase | XM_008461716 |
| CpCPQ | OSC | Cucurbita pepo | Cucurbita pepo CPQ mRNA for | AB116238 |
| | | | cucurbitadienol synthase, complete cds. | |
| CpCPX | OSC | Cucurbita pepo | Cucurbita pepo CPX mRNA for | AB116237 |
| | | | cycloartenol synthase, complete cds. | |
| CqbAS | OSC | Chenopodium quinoa | Chenopodium quinoa isolate CqbAS1 | KX343074 |
| | | | beta-amyrin synthase mRNA, | |
| | | | complete cds. | |
| CsBi/CsCPQ | OSC | Cucumis sativus | cucurbitadienol synthase | KM655855 |
| CsOSC1 | OSC | Costus speciosus | Costus speciosus CSOSC1 mRNA for | AB058507 |
| | | | cycloartenol synthase, complete cds. | |
| CsOSC2 | OSC | Costus speciosus | multi-functional OSC | AB058508 |
| EjAS | OSC | Eriobotrya japonica | multi-functional OSC | |
| EsBAS | OSC | Eleutherococcus | β-amyrin synthase | KX378998 |
| | | senticosus | | |
| EtAS | OSC | Euphorbia tirucalli | β-amyrin synthase | AB206469 |
| GgBAS | OSC | Glycyrrhiza glabra | β-amyrin synthase | AB037203 |
| GgCAS1 | OSC | Glycyrrhiza glabra | cycloartenol synthase | AB025968 |
| GgLUS1 | OSC | Glycyrrhiza glabra | lupeol synthase | AB116228 |
| GsBAS | OSC | Gentiana straminea | β-amyrin synthase | FJ790411 |
| KcCAS | OSC | Kandelia candel | Kandelia candel KcCAS mRNA for | AB292609 |
| | | | cycloartenol synthase, complete cds. | |
| KcMS | OSC | Kandelia candel | Kandelia candel KcMS mRNA for | AB257507 |
| | | | multifunctional triterpene synthase, | |
| | | | complete cds. | |
| KdCAS | OSC | Kalanchoe | Kalanchoe daigremontiana cycloartenol | HM623872 |
| | | daigremontiana | synthase mRNA, complete cds. | |
| KdFRS | OSC | Kalanchoe | Kalanchoe daigremontiana friedelin | HM623870 |
| | | daigremontiana | synthase mRNA, complete cds. | |
| KdGLS | OSC | Kalanchoe | Kalanchoe daigremontiana glutinol | HM623869 |
| | | daigremontiana | synthase mRNA, complete cds. | |
| KdLUS | OSC | Kalanchoe | Kalanchoe daigremontiana lupeol | HM623871 |
| | | daigremontiana | synthase mRNA, complete cds. | |
| KdTAS | OSC | Kalanchoe | Kalanchoe daigremontiana taraxerol | HM623868 |
| | | daigremontiana | synthase mRNA, complete cds. | |
| LaAS1 | OSC | Ilex asprella | Ilex asprella var. asprella mixed amyrin | KM111167 |
| | | | synthase 1 mRNA, complete cds. | |
| LaAS2 | OSC | Ilex asprella | Ilex asprella var. asprella mixed amyrin | KM111168 |
| | | | synthase 2 mRNA, complete cds. | |
| LcCAS1 | OSC | Luffa cylindrica | Luffa cylindrica LcCAS1 mRNA for | AB033334 |
| | | | cycloartenol synthase, complete cds. | |
| LcIMS1 | OSC | Luffa cylindrica | Luffa cylindrica LcIMS1 mRNA for | AB058643 |
| | | | isomultiflorenol synthase, complete cds. | |
| LdCAS1 | OSC | Laurencia dendroidea | Laurencia dendroidea cycloartenol | KX343073 |
| | | | synthase mRNA, complete cds. | |
| LjAMY2 | OSC | Lotus japonicus | Lotus japonicus multifunctional | AF478455 |
| | | | beta-amyrin synthase (AMY2) | |
| | | | mRNA, complete cds. | |
| LjOSC1 | OSC | Lotus japonicus | Lotus japonicus OSC1 mRNA for | AB181244 |
| | | | beta-amyrin synthase, complete cds. | |
| LjOSC3 | OSC | Lotus japonicus | Lotus japonicus OSC3 mRNA for | AB181245 |
| | | | lupeol synthase, complete cds. | |
| LjOSC5 | OSC | Lotus japonicus | Lotus japonicus OSC5 mRNA for | AB181246 |
| | | | cycloartenol synthase, complete cds. | |
| LjOSC7 | OSC | Lotus japonicus | Lotus japonicus OSC7 mRNA for | AB244671 |
| | | | lanosterol synthase, complete cds. | |
| MdOSC1 | OSC | Malus × domestica | multi-functional OSC | FJ032006 |
| MdOSC3 | OSC | Malus × domestica | multi-functional OSC | FJ032008 |
| MdOSC4 | OSC | Malus × domestica | multi-functional OSC | KT383435 |
| MdOSC5 | OSC | Malus × domestica | multi-functional OSC | KT383436 |
| MiCAS | OSC | Maytenus ilicifolia | Maytenus ilicifolia cycloartenol | KX147271 |
| | | | synthase 1 mRNA, complete cds. | |
| MiFRS | OSC | Maytenus ilicifolia | Maytenus ilicifolia Friedelin synthase | KX147270 |
| | | | mRNA, complete cds. | |
| MiFRS(L482V | OSC | Maytenus ilicifolia | mutant OSC | |
| mutant) | | | | |
| MiFRS2 | OSC | Maytenus ilicifolia | | MG677552 |
| MiFRS3 | OSC | Maytenus ilicifolia | | MG677553 |
| MiFRS4 | OSC | Maytenus ilicifolia | | MG677554 |
| MlbAS | OSC | Maesa lanceolata | β-amyrin synthase | KF425519 |
| MtbAS | OSC | Medicago truncatula | β-amyrin synthase | AF478453 |
| NsbAS1 | OSC | Nigella sativa | β-amyrin synthase | FJ013228 |
| ObAS1 | OSC | Ocimum basilicum | β-amyrin synthase | KF636411 |
| ObAS2 | OSC | Ocimum basilicum | multi-functional OSC | JQ809437 |
| OeOEA | OSC | Olea europaea | Olea europaea OEA mRNA for mixed | AB291240 |
| | | | amyrin synthase, complete cds. | |
| OeOEW | OSC | Olea europaea | Olea europaea OEW mRNA for lupeol | AB025343 |
| | | | synthase, complete cds. | |
| OsIAS/OsOSC11 | OSC | Oryza sativa | isoarborinol synthase | AK067451 |
| OsONS1 | OSC | Ononis spinosa | Ononis spinosa onocerin synthase 1 | KY625496 |
| | | | (ONS1) mRNA, complete cds. | |
| OsOSC2 | OSC | Oryza sativa | cycloartenol synthase | AK121211 |
| OsOSC7 | OSC | Oryza sativa | multi-functional OSC | ABG22399 |
| OsOSC8 | OSC | Oryza sativa | multi-functional OSC | AK070534 |
| OsPS/OsOSC7 | OSC | Oryza sativa | parkeol synthase | AK066327 |
| PdFRS | OSC | Populus davidiana | Populus davidiana clone Odea 19 | KY931453 |
| | | | monofunctional friedelin synthase | |
| | | | (FRS) mRNA, complete cds. | |
| PgDDS, PgPNA | OSC | Panax ginseng | dammarenediol-II synthase | AB265170 |
| PgOSCPNX1 | OSC | Panax ginseng | Panax ginseng OSCPNX1 mRNA for | AB009029 |
| | | | Cycloartenol Synthase, complete cds. | |
| PgOSCPNY1 | OSC | Panax ginseng | β-amyrin synthase | AB009030 |
| PgOSCPNY2 | OSC | Panax ginseng | β-amyrin synthase | AB014057 |
| PgPNZ1 | OSC | Panax ginseng | lanosterol synthase | AB009031 |
| PnCAS | OSC | Polypodiodes | Polypodiodes niponica PnCAS mRNA | AB530328 |
| | | niponica | for cycloartenol synthase, complete cds. | |
| PsCASPEA | OSC | Pisum sativum | Pisum sativum CASPEA mRNA for | D89619 |
| | | | cycloartenol synthase, complete cds. | |
| PsOSCPSM | OSC | Pisum sativum | Pisum sativum OSCPSM mRNA for | AB034803 |
| | | | mixed-amyrin synthase, complete cds. | |
| PsOSCPSY | OSC | Pisum sativum | β-amyrin synthase | AB034802 |
| PtbAS | OSC | Polygala tenuifolia | β-amyrin synthase | EF107623 |
| PtOSC | OSC | Phaeodactylum | cycloartenol synthase | Not in GenBank |
| | | tricornutum | | |
| RcCAS | OSC | Ricinus communis | Ricinus communis cycloartenol synthase | DQ268870 |
| | | | mRNA, complete cds. | |
| RcLUS | OSC | Ricinus communis | Ricinus communis lupeol synthase | DQ268869 |
| | | | mRNA, complete cds. | |
| RsCAS | OSC | Rhizophora stylosa | Rhizophora stylosa RsCAS mRNA for | AB292608 |
| | | | cycloartenol synthase, complete cds. | |
| RsM1 | OSC | Rhizophora stylosa | Rhizophora stylosa RsM1 mRNA for | AB263203 |
| | | | multifunctional triterpene synthase, | |
| | | | complete cds. | |
| RsM2 | OSC | Rhizophora stylosa | Rhizophora stylosa RsM2 mRNA for | AB263204 |
| | | | multifunctional triterpene synthase, | |
| | | | complete cds. | |
| SgCBQ | OSC | Siraitia grosvenorii | Siraitia grosvenorii cucurbitadienol | HQ128567 |
| | | | synthase mRNA, complete cds. | |
| SlCAS | OSC | Solanum lycopersicum | cycloartenol synthase | NM_001246855.2 |
| SlTTS1 | OSC | Solanum lycopersicum | β-amyrin synthase | HQ266579 |
| SlTTS2 | OSC | Solanum lycopersicum | multi-functional OSC | HQ266580 |
| SrBOS | OSC | Stevia rebaudiana | Stevia rebaudiana StrBOS mRNA for | AB455264 |
| | | | baccharis oxide synthase, complete cds. | |
| ToTRW | OSC | Taraxacum officinale | Taraxacum officinale TRW mRNA for | AB025345 |
| | | | lupeol synthase, complete cds. | |
| VhBS | OSC | Vaccaria hispanica | Vaccaria hispanica beta-amyrin | DQ915167 |
| | | | synthase (BS) mRNA, complete cds. | |
| WsOSC/BS | OSC | Withania somnifera | Withania somnifera cultivar WS-Y-08 | JQ728553 |
| | | | beta-amyrin synthase mRNA, | |
| | | | complete cds. | |
| WsOSC/CS | OSC | Withania somnifera | Withania somnifera cultivar WS-Y-08 | HM037907 |
| | | | cycloartenol synthase mRNA, | |
| | | | complete cds. | |
| WsOSC/LS | OSC | Withania somnifera | Withania somnifera cultivar WS-Y-08 | JQ728552 |
| | | | lupeol synthase mRNA, complete cds. | |

Preferably, oxidosqualene cyclase is the lupeol synthase OEW from Olea europaea as shown in SEQ ID NO: 6. Said oxidosqualene cyclase as shown in SEQ ID NO: 6 may be encoded by the nucleotide sequence as shown in SEQ ID NO: 22. Other oxidosqualene cyclase preferred in the present invention may refer to Artemissia annua beta-amyrin synthase (AaBAS) as shown in SEQ ID NO: 17 (said enzyme is encoded by the nucleotide sequence as shown in SEQ ID NO: 33) or to Panax ginseng dammarenediol synthase (PgDDS) as shown in SEQ ID NO: 18 (said enzyme is encoded by the nucleotide sequence as shown in SEQ ID NO: 34).

A “cytochrome P450 monooxygenase” means when used in the present invention an enzyme catalysing an enzyme catalysing the oxidation of penta- or tetracyclic triterpenoids by the introduction of an alcohol, aldehyde or acid group at a specific C-atom position. Examples of these enzyme are CYP716A12 as shown in SEQ ID NO: 7 from Medicago truncatula (said enzyme is encoded by the nucleotide sequence as shown in SEQ ID NO: 23), a CYP716A15 as shown in SEQ ID NO: 8 (said enzyme is encoded by the nucleotide sequence as shown in SEQ ID NO: 24) or CYP716A17 as shown in SEQ ID NO: 9 from Vitis vinifera (said enzyme is encoded by the nucleotide sequence as shown in SEQ ID NO: 25) and CYP716A47 as shown in SEQ ID NO: 10 from Panax ginseng (said enzyme is encoded by the nucleotide sequence as shown in SEQ ID NO: 26). Preferably, the cytochrome P450 monooxygenase is CYP716A15 as shown in SEQ ID NO: 8 from Vitis vinifera or CYP716A47 as shown in SEQ ID NO: 10 from Panax ginseng. Other cytochrome P450 monooxygenase may also be comprised in the present invention, as shown by the following table 3:

| Gene | Organism | Enzyme | Genbank ID |
|---|---|---|---|
| AtCYP51 | Arabidopsis thaliana | Obtusifoliol 14a-Demethylase | AY091203 |
| AtCYP51 | Arabidopsis thaliana | Obtusifoliol 14a-Demethylase | AY091203 |
| CYP51H10 | Avena strigosa | β-amyrin C16β-hydroxylase; | DQ680852 |
| | | C12,13 epoxidase | |
| CYP705A1 | Arabidopsis thaliana | arabidiol oxidase | NM_117621 |
| CYP705A12 | Arabidopsis thaliana | Arabidopsis thaliana cytochrome | NM_123622 |
| | | P450, family 705, subfamily A, | |
| | | polypeptide 12 (CYP705A12), mRNA. | |
| CYP705A5 | Arabidopsis thaliana | 7β-Hydroxythalianol | NM_124173 |
| | | C15,16-desaturase | |
| CYP708A2 | Arabidopsis thaliana | thalianol C7-hydroxylase | NM_180821 |
| CYP714E19 | Centella asiatica | C-23 oxidase | KT004520 |
| CYP716A1 | Arabidopsis thaliana | triterpenoid oxidase | NM_123002 |
| CYP716A110 | Aquilegia coerulea | β-amyrin C28-oxidase | KU878864 |
| CYP716A111 | Aquilegia coerulea | β-amyrin C16β-hydroxylase | KY047600 |
| CYP716A113v1 | Aquilegia coerulea | cycloartenol hydroxylase | KU878866 |
| CYP716A12 | Medicago truncatula | pentacyclic triterpenoid C28-oxidase | DQ335781 |
| CYP716A140 | Platycodon | pentacyclic triterpenoid C28-oxidase | KU878853 |
| | grandiflorus | | |
| CYP716A141 | Platycodon | beta-amyrin C16β-hydroxylase | KU878855 |
| | grandiflorus | | |
| CYP716A14v2 | Artemisia annua | pentacyclic triterpenoid C3-oxidase | KF309251 |
| CYP716A15 | Vitis vinifera | pentacyclic triterpenoid C28-oxidase | AB619802 |
| CYP716A154 | Catharanthus roseus | β-amyrin C28-oxidase (CYP716AL1) | JN565975 |
| CYP716A17 | Vitis vinifera | pentacyclic triterpenoid C28-oxidase | AB619803 |
| CYP716A175 | Malus × domestica | β-amyrin C28-oxidase | XM_008392874 |
| CYP716A179 | Glycyrrhiza uralensis | pentacyclic triterpenoid C28-oxidase | LC157867 |
| CYP716A2 | Arabidopsis thaliana | multifunctional pentacyclic | LC106013 |
| | | triterpenoid oxidase | |
| CYP716A244 | Eleutherococcus | β-amyrin C28-oxidase | KX354739 |
| | senticosus | | |
| CYP716A252 | Ocimum basilicum | β-amyrin C28-oxidase | JQ958967 |
| CYP716A253 | Ocimum basilicum | β-amyrin C28-oxidase | JQ958968 |
| CYP716A254 | Anemone flaccida | β-amyrin C-28-oxidase | |
| CYP716A44 | Solanum | pentacyclic triterpenoid C28-oxidase | AK329870 |
| | lycopersicum | | |
| CYP716A46 | Solanum | pentacyclic triterpenoid C28-oxidase | XM_004243858 |
| | lycopersicum | | |
| CYP716A52v2 | Panax ginseng | β-amyrin C28-oxidase | JX036032 |
| CYP716A75 | Maesa lanceolata | β-amyrin C28-oxidase | KF318733 |
| CYP716A78 | Chenopodium | β-amyrin C28-oxidase | KX343075 |
| | quinoa | | |
| CYP716A79 | Chenopodium | β-amyrin C28-oxidase | KX343076 |
| | quinoa | | |
| CYP716A80 | Barbarea vulgaris | pentacyclic triterpenoid C28-oxidase | KP795926 |
| CYP716A81 | Barbarea vulgaris | pentacyclic triterpenoid C28-oxidase | KP795925 |
| CYP716A83 | Centella asiatica | pentacyclic triterpenoid C28-oxidase | KU878849 |
| CYP716A86 | Centella asiatica | pentacyclic triterpenoid C28-oxidase | KU878848 |
| CYP716C11 | Centella asiatica | oleanolic acid C2α-hydroxylase | KU878852 |
| CYP716E26 | Solanum | C-6beta-hydroxylase | XM_004241773 |
| | lycopersicum | | |
| CYP716E41 | Centella asiatica | maslinic acid C6β-hydroxylase | KU878851 |
| CYP716S1 | Panax ginseng | protopanaxadiol C6-hydroxylase | JX036031 |
| | | (CYP716A53v2) | |
| CYP716S5 | Platycodon | beta-amyrin C12,13α-epoxidase | KU878856 |
| | grandiflorus | | |
| CYP716U1 | Panax ginseng | dammarenediol-II C12-hydroxylase | JN604536 |
| | | (CYP716A47v1) | |
| CYP716Y1 | Bupleurum falcatum | pentacyclic triterpenenoid | KC963423 |
| | | C16α-hydroxylase | |
| CYP71A16 | Arabidopsis thaliana | marneral C23-hydroxylase | NM_123623 |
| CYP71D353 | Lotus japonicus | dihydrolupeol oxidase | KF460438 |
| CYP71D443 | Ajuga reptans | sterol 22-hydroxylase | LC066937 |
| CYP72A154 | Glycyrrhiza uralensis | oleanane 30-oxidase | AB558153 |
| CYP72A61v2 | Medicago truncatula | 24-hydroxy-β-amyrin- | AB558145 |
| | | C-22β-hydroxylase | |
| CYP72A63 | Medicago truncatula | β-amyrin C30-oxidase | AB558146 |
| CYP72A67 | Medicago truncatula | oleanoilc acid C-2β-hydroxylase | DQ335780 |
| CYP72A68v1 | Medicago truncatula | oleanane C-23-oxidase | XM_013608494 |
| CYP72A68v2 | Medicago truncatula | oleanane C-23-oxidase | DQ335782 |
| CYP72A69 | Glycine max | soyasapogenol C-21β-hydroxylase | LC143440 |
| CYP734A1 | Arabidopsis thaliana | brassinosteroid C26-hydroxylase | BT010564 |
| CYP734A7 | Solanum | brassinosteroid C26-hydroxylase | AB223041 |
| | lycopersicum | | |
| CYP76AH15 | Plectranthus | diterpenoid oxidase | KT382358 |
| | barbatus | | |
| CYP81Q58 | Citrullus lanatus | Cucurbitane C-25-oxidase | Cla007077 |
| CYP81Q58 | Cucumis sativus | Cucurbitane C-25-oxidase | KM655862 |
| CYP81Q59 | in three plants | Cucurbitane C-2-hydroxylase | |
| CYP85A1 | Arabidopsis thaliana | brassinosteroid 26-oxidase | NM_001344281 |
| CYP85A2 | Arabidopsis thaliana | brassinosteroid 26-oxidase | NP_566852 |
| CYP87D16 | Maesa lanceolata | β-amyrin C-16α-hydroxylase | KF318735 |
| CYP87D18 | Siraitia grosvenorii | cucurbitane C-11-oxidase | HQ128570 |
| CYP88D6 | Glycyrrhiza uralensis | oleanane C-11-oxidase | AB433179 |
| CYP88L2 | Cucumis sativus | cucurbitane C-19-hydroxylase | |
| CYP93E1 | Glycine max | oleanane C-24-oxidase | AF135485 |
| CYP93E2 | Medicago truncatula | β-amyrin C-24-oxidase | DQ335790 |
| CYP93E3 | Glycyrrhiza uralensis | β-amyrin C-24-oxidase | AB437320 |
| CYP93E4 | Arachis hypogaea | β-amyrin C-24-oxidase | KF906535 |
| CYP93E5 | Cicer arietinum | β-amyrin C-24-oxidase | KF906536 |
| CYP93E6 | Glycyrrhiza glabra | β-amyrin C-24-oxidase | KF906537 |
| CYP93E7 | Lens culinaris | β-amyrin C-24-oxidase | KF906538 |
| CYP93E8 | Pisum sativum | β-amyrin C-24-oxidase | KF906539 |
| CYP93E9 | Phaseolus vulgaris | β-amyrin C-24-oxidase | KF906540 |
| CYP94N1 | Veratrum | sterol C-26-hydroxylase | KJ869255 |
| | californicum | | |
| NtCYP51 | Nicotiana tabacum | Obtusifoliol 14a-Demethylase | AF116915 |
| PsCYP51 | Pisum sativum | Obtusifoliol 14a-Demethylase | AB633330 |
| SbCYP51 | Sorghum bicolor | Obtusifoliol 14a-Demethylase | U74319 |
| SlCYP85A1 | Solanum | brassinosteroid 26-oxidase | SLU54770 |
| | lycopersicum | | |
| TaCYP51 | Trticum aestivum | Obtusifoliol 14a-Demethylase | Y09291 |
| ZmCYP51 | Zea mays | Obtusifoliol 14a-Demethylase | |

The term “cytochrome P450 reductase” describes enzymes transferring electrons from NADH to cytochrom450. The term includes when used in the context of the present invention, but is not limited to, ATR1 as shown in SEQ ID NO: 11 (said enzyme is encoded by the nucleotide sequence as shown in SEQ ID NO: 27) or ATR2 as shown in SEQ ID NO: 12 from Arabidopsis thallena (said enzyme is encoded by the nucleotide sequence as shown in SEQ ID NO: 28), or LjCPR1 as shown in SEQ ID NO: 13 from Lotus japonicus (said enzyme is encoded by the nucleotide sequence as shown in SEQ ID NO: 29), or CrCRP as shown in SEQ ID NO: 14 from Catharanthus roseus (said enzyme is encoded by the nucleotide sequence as shown in SEQ ID NO: 30).

In the context of the present invention the term “sterol acyltransferase” is in general an enzyme mediating the chemical reaction of forming sterol ester from sterol by using acyl-CoA. Said sterol acyltransferase may refer to the enzyme as shown in SEQ ID NO: 15 or 16.

In a preferred embodiment the present invention encompasses a yeast strain comprising an Erg7p protein combined with the decay sequence of SEQ ID NO: 1, as well as a gene integration of at least one polynucleotide encoding a cytochrome P450 monooxygenase, and/or at least one polynucleotide encoding a lupeol synthase, beta-amyrin synthase or dammarenediol synthase. Even more preferred, the yeast comprises an Erg7p protein combined with the decay sequence of SEQ ID NO: 1, as well as a gene integration of at least one polynucleotide encoding a cytochrome P450 monooxygenase which is CYP716A15 as shown in SEQ ID NO: 8 from Vitis vinifera or CYP716A47 as shown in SEQ ID NO: 10 from Panax ginseng, and/or at least one polynucleotide encoding a lupeol synthase OEW from Olea europaea as shown in SEQ ID NO: 6. It is envisaged by the present invention that the at least one gene integration includes two, three, four, five or even a higher number of gene integrations for each of the above mentioned polynucleotides.

Further encompassed herein is the combination of Erg1p, or Erg9p, or Erg20p with the decay sequence as well as a gene integration of at least one polynucleotide encoding a cytochrome P450 monooxygenase, and/or at least one polynucleotide encoding a lupeol synthase, beta-amyrin synthase or dammarenediol synthase as described and further defined elsewhere herein. Also in this context it is envisaged by the present invention that the at least one gene integration includes two, three, four, five or even a higher number of gene integrations for each of the above mentioned polynucleotides.

### Method for Production

The present invention further relates to a method for the production of a triterpenoid and derivatives thereof, comprising culturing a host cell which may be enriched with the vector comprising the expression cassette, or the expression cassette itself as defined elsewhere herein, under conditions which allow the production of a triterpenoid; and harvesting the triterpenoid produced by the host cell.

The term “triterpenoid” as used herein means a chemical compound belonging to the class of terpenes, which are structurally composed of units of isoprene. The molecular formula of isoprene is C₅H₈, the basic molecular formulas of terpenes are hence multiples of (C₅H₈)n, where n is the number of linked isoprene units. Betulinic acid for example as described elsewhere herein may refer to a triterpenoid. To reach high triterpenoid production yields it is preferred that the lanosterol synthase transforming 2,3-oxidosqualene to lanosterol is downregulated according to the present invention. However, other enzymes stated in (i) to (vi) are preferably very active to ensure high triterpenoid production yields.

In the present invention “culturing conditions” refer to conditions which allow the production of triterpenoids when using the host cell according to the present invention. The production of triterpenoids and derivatives thereof is described as follows.

Host cells of the present invention are preferably grown either in complex medium (e.g. YPD) or in mineral salt medium (e.g. SC medium, WM8+ medium, MCA medium) at about 30° C. Positive transformants are screened on SC (synthetic complete) medium lacking appropriate amino acids or in YPD with appropriate antibiotics. Even more preferably, production of triterpenoids in shake flasks is performed by growing the strains in WM8+ medium as described in the following: glucose 50 g/L, NH₄H₂PO₄ 0.25 g/L, NH₄Cl 2.8 g/L, sodium glutamate 10 g/L, MgCl₂·6H₂O 0.25 g/L, CaCl₂·2H₂O 0.1 g/L, KH₂PO₄ 2 g/L, MgSO₄·7H₂O 0.55 g/L, myo-inositol 100 mg/L, ZnSO₄·7H₂O 6.25 mg/L, FeSO₄·7H₂O 3.5 mg/L, CuSO₄·5H₂O 0.4 mg/L, MnCl₂·4H₂O 0.1 mg/L, MnCl₂·2H₂O 1 mg/L, Na₂MoO₄·2H₂O 0.5 mg/L, CoCl₂·6H₂O 0.3 mg/L, H₃BO₃ 1 mg/L, KI 0.1 mg/L, nicotinic acid 11 mg/L, pyridoxin-HCl 26 mg/L, thiamine-HCL 11 mg/L, biotin 2.55 mg/L, calcium pantothenate 51 mg/L, p-aminobenzoic acid 0.2 mg/L, and Na₂EDTA 3.36 g/L.

A “derivate” according to the present invention is a compound that is formed from a similar compound, or a compound that can be imagined to arise from another compound, if one atom is replaced with another atom or group of atoms. In biochemistry, the word is used for compounds that at least theoretically can be formed from the precursor compound. For example, a triterpenoid derivate may include, but is not limited to oleanane, ursolic acid, or lanostane.

The term “harvesting” is herein related to the isolation of triterpenoid and derivatives thereof, produced by said host cell. In this context, it is meant that after the triterpenoid compound and/or triterpenoid derivatives accumulate in the cell, the cell is physically disrupted by destroying the cell membrane of the cell. After disruption of the host cell as defined herein, either the medium (e.g. entire fermentation broth) is extracted which then comprises the triterpenoid and derivatives thereof, produced by said host cell without a centrifugation step, or the triterpenoid and derivatives thereof produced by said host cell are isolated after a centrifugation step from said supernatant. According to the present invention, when the term “harvesting” is used it preferably refers to disrupting the host cell and then extract the medium which then comprises the triterpenoid and derivatives thereof produced by said host cell without a centrifugation step.

Conveniently, the compound can be harvested from the culture medium, lysates of the cultured host cell or from isolated (biological) membranes by established techniques. For example, the product may be recovered from the host cell and/or culture medium by conventional procedures including, but not limited to, cell lysis, breaking up host cells, centrifugation, filtration, ultra-filtration, extraction or precipitation. Purification may be performed by a variety of procedures known in the art including, but not limited to, chromatography (e.g. ion exchange, affinity, hydrophobic, chromatofocusing, and size exclusion), electrophoretic procedures (e.g. preparative isoelectric focusing), differential solubility (e.g. ammonium sulfate precipitation) or extraction.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. The term “at least one” refers, if not particularly defined differently, to one or more such as two, three, four, five, six, seven, eight, nine, ten or more. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number.

For example, less than 20 means less than the number indicated. Similarly, more than or greater than means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

The term “about” means plus or minus 10%, preferably plus or minus 5%, more preferably plus or minus 2%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc. described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be gained from the following examples, offered for illustrative purposes only. The examples are not intended to limit the scope of the present invention in any way.

### Examples of the Invention

The following Examples illustrate the invention, but are not to be construed as limiting the scope of the invention.

### Example 1: Modification of the Frameshift Mutant ERG7 in Simo1575.

To test the influence of the disrupted tag (tGFP-cODC1-TDegF-RFP; tGFP, truncated GFP) downstream of the frameshift in ERG7, several strains were created, in which different parts of the tGFP-cODC1-TDegF-RFP degron in Simo1575 were replaced by a bleR cassette (see as illustrated in SEQ ID NO: 41), yielding Simo1575-o-ERG7 (amino acid sequence SEQ ID NO: 39 and nucleotide sequence SEQ ID NO: 40), Simo1575-t-ERG7 (amino acid sequence SEQ ID NO: 19 and nucleotide sequence SEQ ID NO: 35), Simo1575-m-ERG7 (amino acid sequence SEQ ID NO: 20 and nucleotide sequence SEQ ID NO: 36), and Simo 1575-gt-ERG7 strain (amino acid sequence SEQ ID NO: 37 and nucleotide sequence SEQ ID NO: 38; disrupted GFP, CA motif and part of the spacer upstream of the TEV protease recognition site, respectively (FIG. 2A).

To further verify that the frameshift ERG7 caused 2,3-oxidosqualene accumulation in the Simo1575 strain, the fragment containing the construct (tGFP-cODC1-TDegF-RFP) and upstream ERG7 mutant gene was replaced in Simo1575 strain by the yeast's native ERG7 gene and the bleR cassette, resulting in strain Simo1575+ERG7. All strains were cultivated in YPD medium with 2% glucose for 72 h. As shown in FIGS. 2B and 2C, Simo1575, Simo1575-gt-ERG7, Simo1575-m-ERG7, Simo1575-t-ERG7 and Simo1575-o-ERG7 accumulated high titres of 2,3-oxidosqualene. Interestingly, the specific 2,3-oxidosqualene titre in Simo1575-gt-ERG7, Simo1575-m-ERG7, Simo1575-t-ERG7 and Simo1575-o-ERG7 increased by 2-fold compared to Simo1575, reaching up to 65 mg/g cell dry weight (FIGS. 2B and 2C). In contrast and in line with our assumption, the Simo1575+ERG7 strain did not accumulate 2,3-oxidosqualene. These results indicate that the frameshift ERG7 gene is responsible for producing 2,3-oxidosqualene in the Simo1575 strain.

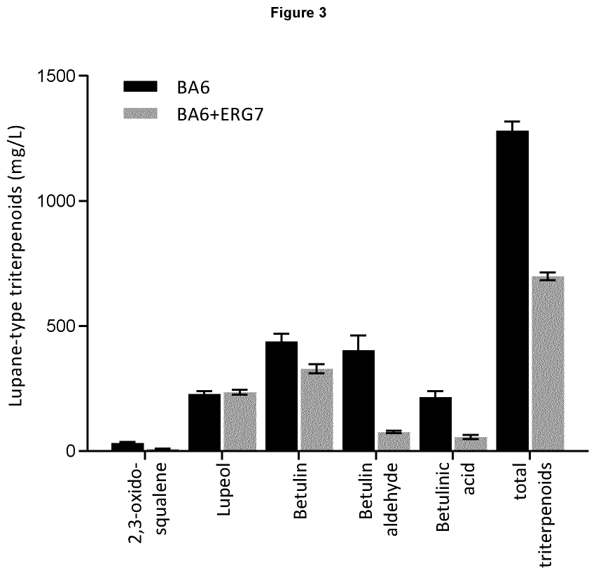

The strain BA6 (see Table 4), a strain originating from Simo1575 and engineered for betulinic acid production had been shown to accumulate significantly higher triterpenoid titres compared with a similarly engineered S. cerevisiae strain lacking the ERG7 mutation, described in Czarnotta et al., during batch cultivation. To verify that this improvement is caused by the frameshift mutation in the ERG7 gene, the mutant ERG7 sequence was replaced by the native ERG7 in the BA6 strain (BA6+ERG7). The recombinant strain BA6+ERG7, relative to BA6, produced significantly fewer triterpenoids with about 330 mg/L betulin, 70 mg/L betulinic aldehyde, and 50 mg/L betulinic acid, summing up to a total titre of triterpenoids that is half to that of BA6 (FIG. 3).

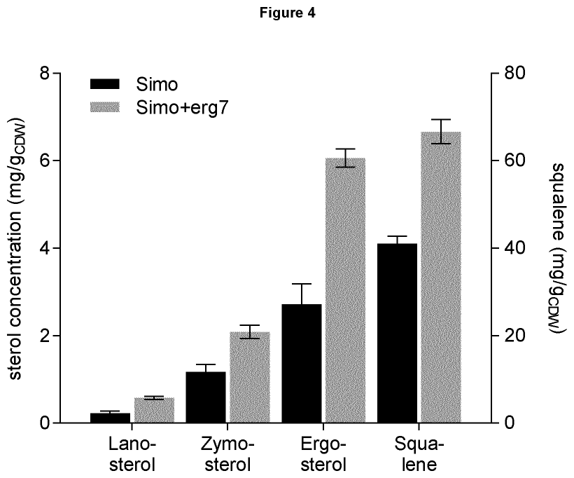

These data indicated that the frameshift facilitates carbon flow towards triterpenoids production. The mutant ERG7 gene in Simo1575 strain resulted in 2,3-oxidosqualene accumulation and boosted triterpenoids production in BA6. Meanwhile, lanosterol synthase (encoded by ERG7) catalyses the essential step for ergosterol biosynthesis. Thus, the effect of mutant Erg7p on the downstream sterols in the post-squalene pathway was assessed. Simo1575 strain exhibited a 2-fold decrease in lanosterol, zymosterol, ergosterol, and squalene compared with Simo1575+ERG7 (FIG. 4). These data suggested that the frameshift decreased the carbon flux into the ergosterol biosynthesis pathway in Simo1575, thereby fostering triterpenoid synthesis.

### Example 2: Reconstruction of 2,3-Oxidosqualene Producer Strain

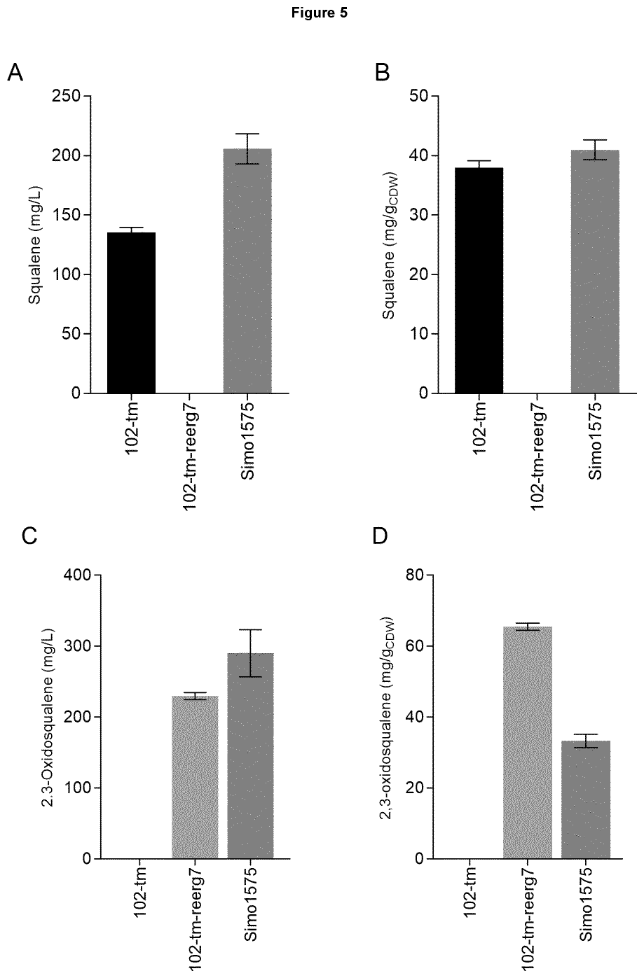

To construct a triterpenoid chassis, the design of the 2,3-oxidosqualene producer strain was reconstructed in S. cerevisiae CEN.PK 102-5B, the same basic strain as used for the development of Simo1575. First, to increase the metabolic flux in the mevalonate pathway and prepare for subsequent P450 reactions, one copy of the tHMG1 (N-terminal truncated HMG-COA reductase gene) gene and the MTR (P450 reductase from Medicago truncatula) gene were integrated into chromosome X of S. cerevisiae CEN.PK 102-5B. The cytochrome P450 reductase MTR has previously been reported to be superior for the conversion of lupeol to betulinic acid in S. cerevisiae (US20170130233A, patent). The tHMG1 and MTR genes were expressed under the control of the constitutive promoters P_{PGK1} and P_{TEF1}, respectively (see as illustrated in SEQ ID NO: 44). The resulting recombinant strain, named 102-tm, accumulated about 137 mg/L squalene with a specific titre of 39 mg/g cell dry weight in YPD medium with 2% glucose (FIG. 5A, B).

This result confirms again that the tHMG1 gene modification leads to an increase in the metabolic flux into the mevalonate pathway (Polakowski, Stahl et al., 1998). Moreover, to engineer a frameshift mutant ERG7 in strain 102-tm, a fragment containing the HphMX6 expression cassette was fused to the C-terminus of the frameshift ERG7 mutant in strain 102-tm. As shown in FIG. 5, the resulting strain 102-tm-reerg7 produced 2,3-oxidosqualene instead of squalene, reaching up to 227 mg/L, while no squalene was observed. The reconstructed 2,3-oxidosqualene-producing strain 102-tmreerg7 and strain Simo1575 accumulated similar amounts of 2,3-oxidosqualene. However, strain 102-tm-reerg7 displayed a higher specific titre of 2,3-oxidosqualene of 66 mg/g cell dry weight, about a 2-fold improvement compared to strain Simo1575. These data indicated that the frameshift mutant ERG7 could result in the 2,3-oxidosqualene accumulation merely with overexpression of the tHMG1 gene. Also, these results suggested that the repression of ERG1 is relieved in the presence of the frameshift mutant ERG7.

Example 3: Production of Lupane-Type Triterpenoids in the Reconstructed 2,3-oxidosqualene Accumulating Strain.

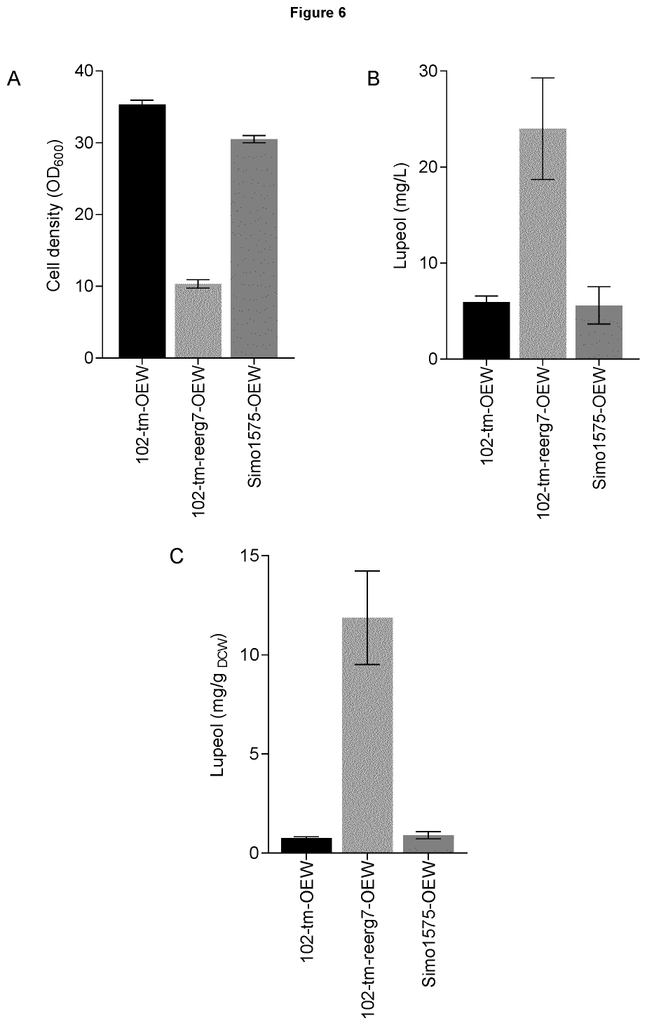

The 2,3-oxidosqualene producer strain 102-tm-reerg7 was investigated according to the effect of accumulated 2,3-oxidosqualene on the production of lupeol. Therefore, one copy of the OEW gene (lupeol synthase from Olea europaea) was integrated into the chromosome of the 102-tm, 102-tm-reerg7, and Simo1575 strain (see as illustrated in SEQ ID NO: 44). The recombinant strains were designated as 102-tmOEW, 102-tm-reerg7-OEW and Simo1575-OEW, respectively. As depicted in FIG. 6, during shake flask cultivation using YPD medium, strain 102-tm-reerg7-OEW unexpectedly produced much less biomass than 102-tm-OEW, however, the specific and volumetric titre of lupeol in 102-tm-reerg7-OEW is 16-fold higher than that of 102-tm-OEW, reaching 12 mg/g cell dry weight and 24 mg/L, respectively. Overexpression of the OEW gene in strain Simo1575 did not cause a similar growth deficiency, but the strain accumulated less lupeol than 102-tm-reerg7-OEW. These data suggested that accumulation of 2,3-oxidosqualene can still improve the production of lupeol despite the apparent growth deficiency.

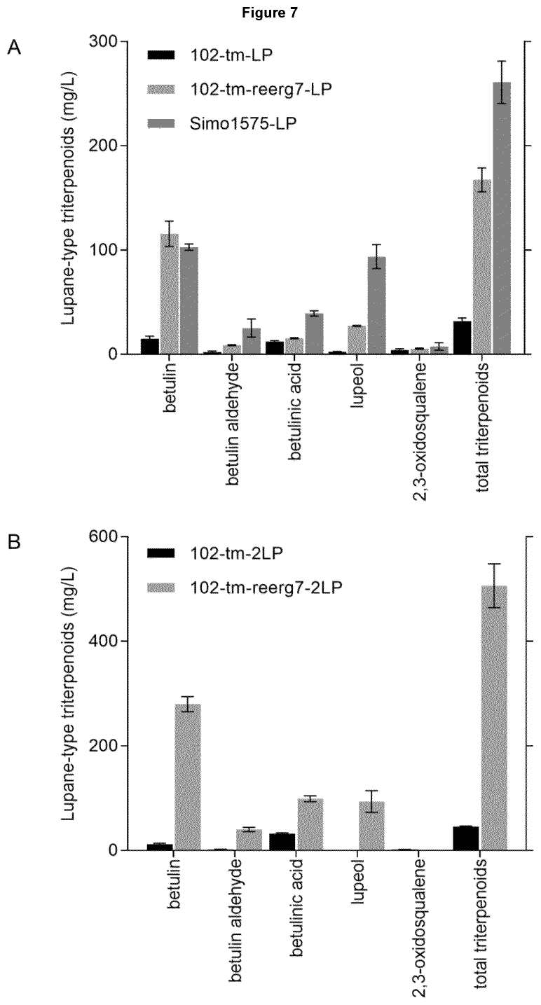

To further investigate the impact of accumulated 2,3-oxidosqualene on the production of betulin, betulin aldehyde, and betulinic acid, one copy of the OEW (lupeol synthase from Olea europaea) and CYP716A15 (P450s gene from Vitis vinifera) genes were integrated into the chromosome of the strains 102-tm and 102-tm-reerg7. The recombinant strains were named 102-tm-LP and 102-tm-reerg7-LP, respectively. Strain 102-tm-reerg7-LP can produce 111 mg/L betulin, 9 mg/L betulin aldehyde, 16 mg/L betulinic acid, 28 mg/L lupeol, and 163 mg/L total triterpenoids in YPD medium with 2% glucose. In contrast, strain 102-tm-LP can only produce 16 mg/L betulin, 3 mg/L betulinic aldehyde, 12 mg/L betulinic acid, 3 mg/L lupeol, a total of 33 mg/L triterpenoids. Hence, strain 102-tm-reerg7-LP exhibited an overall improvement in the production of lupane-type triterpenoids compared with strain 102-tm-LP (FIG. 7). It was also examined whether the reconstructed 2,3-oxidosqualene producing strain is distinct from Simo1575 concerning the production of betulin, betulin aldehyde, and betulinic acid. Hence, one copy of OEW and CYP716A15 genes were integrated into the chromosome of strain Simo1575 (URA3-), resulting in strain Simo1575-LP. Simo1575-LP produced 101 mg/L betulin, 27 mg/L betulinic aldehyde, 40 mg/L betulinic acid, 94 mg/L and 261 mg/L total lupane-type triterpenoids, which were about 1.6-fold higher than 102-tm-reerg7-LP. These results suggested that Simo1575 is better at producing betulin, betulinic aldehyde, and betulinic acid than 102-tm-reerg7.

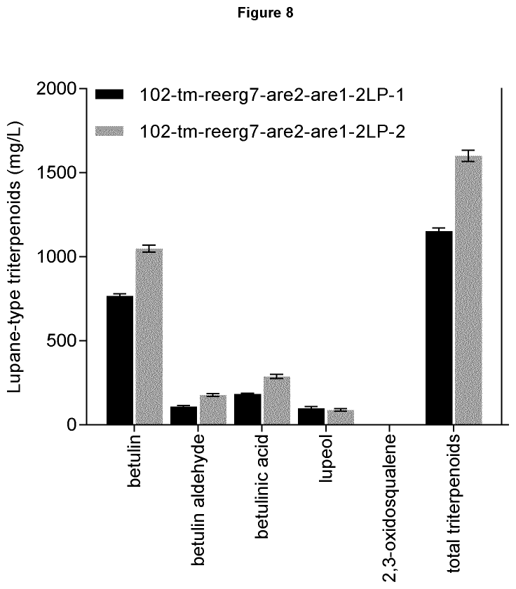

As shown in FIG. 3, high-level production of lupane-type triterpenoids can be achieved with strain BA6 (multicopy genomic integration of the OEW gene and CYP716A15 gene on the chromosome of Simo1575) reaching total triterpenoid titres above 1.5 g/L in shake-flask experiments with WM8+medium, significantly higher tires than those of 102-tm-reerg7-LP. Hence, it was surmised that the low activity of the lupeol synthase and CYP716A15 achievable with the single-copy integration of these genes might be mainly limiting the production of the triterpenoids in the reversed engineered strains. To prove this hypothesis, another copy of OEW and P450 genes was chromosomally integrated into 102-tm-LP and 102-tm-reerg7-LP, resulting in strains 102-tm-2LP and 102-tm-reerg7-2LP, respectively. 102-tm-re-2LP was able to produce 520 mg/L total triterpenoids (including 285 mg/L betulin, 40 mg/L betulin aldehyde and 101 mg/L betulinic acid), which were 11-fold higher than the respective titres in strain 102-tm-2LP (FIG. 7). These results demonstrated that the activity of lupeol synthase and CYP716A15 were limiting lupane-type triterpenoid production. Total triterpenoid titres comparable to those determined with the strain BA6 were achieved after deletion of the acyl transferase encoding genes and cultivation in WM8+medium (FIG. 8)

### Example 4: Evaluation of the ERG7 Decay Strain as Triterpenoid Production Platform.

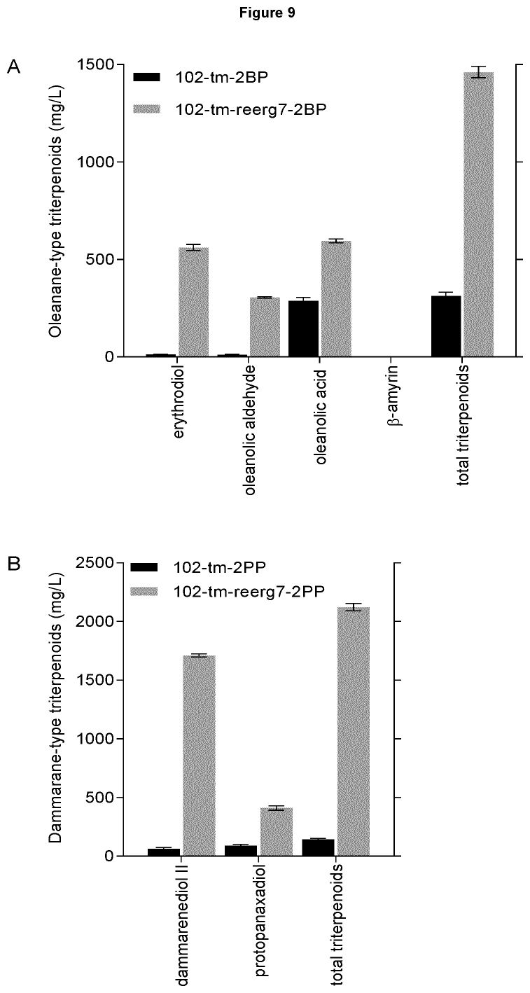

To test the impact of cumulative 2,3-oxidosqualene on the oleanolic acid biosynthetic pathway in 2,3-oxidosqualene producer strain, two copies of the AaBAS gene (encoding β-amyrin synthase from Artemisia annua, SEQ ID NO: 17) and CYP716A15 (P450 monooxygenase from Vitis vinifera) were integrated into the chromosome of strain 102-tm-reerg7 and 102-tm, respectively. The AaBAS and CYP716A15 genes were respectively controlled by the constitutive promoters, P_{PGK1} and P_{TEF1} (see as illustrated in SEQ ID NO: 44). The recombinant strains were designated as 102-tm-re2BP and 102-tm-2BP. Strains 102-tmre2BP 102-tm-2BP exhibited longer lag phases than other strains, which is in agreement with Kirby, Romanini et al., 2008. The β-amyrin peak was not detected in the HPLC-CAD analysis. However, strain 102-tm-reerg7-2BP can produce 1466 mg/L total oleanane-type triterpenoids (including 564 mg/L erythrodiol, 306 mg/L oleanolic aldehyde, 596 mg/L oleanolic acid), which was 459% higher than that of strain 102-tm-2BP (FIG. 9).

Similarly, in order to investigate the effect of accumulated 2,3-oxidosqualene on the protopanaxadiol biosynthetic pathway in the 2,3-oxidosqualene producer strain, two strains, 102-tm-reerg7-2PP and 102-tm-2PP were obtained by integration of two copies of PgDDS (encoding dammarenediol synthase from Panax ginseng SEQ ID NO: 18) and CYP716A47 (P450 monooxygenase from Panax ginseng) in the 102-tm-reerg7 and 102-tm. The 102-tm-re2PP strain accumulated 2,117 mg/L total dammarene-type triterpenoids (including 1,713 mg/L dammarenediol, 404 mg/L protopanaxadiol), about a 14.4-fold improvement compared with strain 102-tm-2PP. These results indicated that accumulated 2,3-oxidosqualene in 102-tm-reerg7 could dramatically improve the titre of tetracyclic and pentacyclic triterpenoids (FIG. 9).

TABLE 4
Yeast strains used in the present invention:

| Strain | Description |
|---|---|
| Simo1575 | S. cerevisiae CEN.PK 102-5B, the cassettes p_{URA3}-URA3, p_{CUP1}-TEV and |
| | P_{TEF1}-tHMG1--P_{PGK1}-ATR2 were integrated as single copies into chromosome |
| | X, are2::kanMX; ERG7::ODCtag::GFP::ENLYFQ-F::RFP, multi-copy integration |
| | of OEW and CYP716A15 into TY4 sites. |
| Simo1575-gt-ERG7 | Partial removal of the disrupted degron tGFP-cODC1-TDegF-RFP in Simo1575 |
| Simo1575-m-ERG7 | Partial removal of the disrupted degron tGFP-cODC1-TDegF-RFP in Simo1575 |
| Simo1575-t-ERG7 | Partial removal of the disrupted degron tGFP-cODC1-TDegF-RFP in Simo1575 |
| Simo1575-o-ERG7 | Removal of the disrupted degron tGFP-cODC1-TDegF-RFP in Simo1575 |
| Simo1575 + ERG7 | Replacement of the mutated ERG7 gene and downstream part of the degron |
| | (tGFP-cODC1-TDegF-RFP) with the yeast native ERG7 gene in Simo1575 |
| Simo1575-LP | Integration of P_{PGK1}-OEW-T_{CYC1}, P_{TEF1} -CYP716A15-T_{ADH1} cassettes and URA3 |
| | marker gene into chromosomes XI of Simo1575 |
| BA6 | Integration of P_{PGK1}-OEW-T_{CYC1}, P_{TEF1}-CYP716A15-T_{ADH1} cassettes and URA3 |
| | marker gene into chromosomes XI of Simo1575 (URA3-) |
| BA6 + ERG7 | Replacement of the mutated ERG7 gene and downstream part of the degron |
| | (cODC1-TDegF-RFP with the disrupted GFP) with the yeast native ERG7 gene in BA6 |
| 102-tm | Integration of P_{TEF1}-tHMG1-T_{ADH1}, P_{PGK1}-MTR-T_{CYC1} cassettes and LEU2 marker |
| | gene into chromosomes X of S. cerevisiae CEN.PK 102-5B |
| 102-tm-reerg7 | Replacement of the native ERG7 gene with the frame-shift ERG7 mutant in 102-tm |
| 102-tm-OEW | Integration of P_{PGK1}-OEW-T_{CYC1} cassettes and URA3 marker gene into |
| | chromosome XI of 102-tm |
| 102-tm-reerg7-OEW | Integration of P_{PGK1}-OEW-T_{CYC1} cassettes and URA3 marker gene into |
| | chromosome XI of 102-tm-reerg7 |
| 102-tm-2LP | Integration of P_{PGK1}-OEW-T_{CYC1}, P_{TEF1}-CYP716A15-T_{ADH1} cassettes and HIS3 marker |
| | gene into chromosomes XII and P_{PGK1}-OEW-T_{CYC1}, P_{TEF1}-CYP716A15-T_{ADH1} |
| | cassettes and URA3 marker gene into chromosome XI of 102-tm |
| 102-tm-reerg7-2LP | Integration of P_{PGK1}-OEW-T_{CYC1}, P_{TEF1}-CYP716A15-T_{ADH1} cassettes and HIS3 marker |
| | gene into chromosome XII and P_{PGK1}-OEW-T_{CYC1}, P_{TEF1}-CYP716A15-T_{ADH1} |
| | cassettes and URA3 marker gene into chromosome XI of 102-tm-reerg7 |
| 102-tm-reerg7- | Deletion of ARE1 and ARE2 in 102-tm-reerg7-2LP |
| are2-are1-2LP | |
| 102-tm-2BP | Integration of P_{PGK1}-AaBAS-T_{CYC1}, P_{TEF1}-CYP716A15-T_{ADH1} cassettes and HIS3 |
| | marker gene into chromosome XII and P_{PGK1}-AaBAS-T_{CYC1}, P_{TEF1}-CYP716A15-T_{ADH1} |
| | cassettes and URA3 marker gene into chromosome XI of 102-tm |
| 102-tm-reerg7-2BP | Integration of P_{PGK1}-AaBAS-T_{CYC1}, P_{TEF1}-CYP716A15-T_{ADH1} cassettes and HIS3 |
| | marker gene into chromosome XII and P_{PGK1}-AaBAS-T_{CYC1}, P_{TEF1}-CYP716A15-T_{ADH1} |
| | cassettes and URA3 marker gene into chromosome XI of 102-tm |
| 102-tm-2PP | Integration of P_{PGK1}-PgDDS-T_{CYC1}, P_{TEF1}-CYP716A49-T_{ADH1} cassettes and HIS3 |
| | marker gene into chromosome XII and P_{PGK1}-PgDDS-T_{CYC1}, P_{TEF1}-CYP716A49-T_{ADH1} |
| | cassettes and URA3 marker gene into chromosome XI of 102-tm |
| 102-tm-reerg7-2PP | Integration of P_{PGK1}-PgDDS-T_{CYC1}, P_{TEF1}-CYP716A49-T_{ADH1} cassettes and HIS3 |
| | marker gene into chromosome XII and P_{PGK1}-PgDDS-T_{CYC1}, P_{TEF1}-CYP716A49-T_{ADH1} |
| | cassettes and URA3 marker gene into chromosome XI of 102-tm. |

### REFERENCES

Altschul, S. F., Gish, W., Miller, W., Myers, E. W., and Lipman, D. J. (1990). “Basic local alignment search tool.” J Mol Biol, 215:403-410.
Altschul, S. F., Madden, T. L., Schaffer, A. A., Zhang, J., Zhang, Z., Miller, W., and Lipman, D. J. (1997). “Gapped BLAST and PSI-BLAST: a new generation of protein database search programs” Nucleic Acid Research, 25 (17): 3389-3402.
Czarnotta, E., Dianat, M., Korf, M., Granica, F., Merz, J., Maury, J., Baallal Jacobsen, S. A., Forster, J., Ebert, B. E., Blank, L. M. (2017). “Fermentation and purification strategies for the production of betulinic acid and its lupane-type precursors in Saccharomyces cerevisiae”. Biotechnol Bioeng, 114, 2528-2538. Kalaivani and Sarma (2017). “Progress in terpene synthesis strategies through engineering of S. cerevisiae”. CRC Critical Reviews in Biotechnology, vol. 37, no. 8.
Kirby, J., Romanini, D. W., Paradise E. M., and Keasling, J. D. (2008). “Engineering triterpene production in Saccharomyces cerevisiae-beta-amyrin synthase from Artemisia annua.” Febs J, 275 (8): 1852-1859.
Knuf et al. (2014). “Application of a controllable degron strategy for metabolic engineering”. New Biotechnology, vol. 31.
Peng et al. (2018). “Engineered protein degradation of farnesyl pyrophosphate synthase is an effective regulatory mechanism to increase monoterpene production in S. cerevisiae”. Metabolic Engineering, Academic Press, vol. 47.
Polakowski, T., Stahl, U., and Lang, C. (1998). “Overexpression of a cytosolic hydroxymethylglutaryl-CoA reductase leads to squalene accumulation in yeast.” Appl Microbiol Biotechnol, 49 (1): 66-71.
Suzuki and Varshavsky (1999). “Degradation signals in the lysine-asparagine sequence space”. The EMBO Journal, vol. 18, no. 21, pp. 6017-6026.
Wang, Y., O'Malley Jr, B. W., Tsai, S. Y., and O'Malley, B. W. (1994). “A regulatory system for use in gene transfer.” Proc. Natl. Acad. Sci. USA, 91, 8180-8184.
Wang, P., Wei, W., Ye, W., Li, X., Zhao, W., Yang, C., Li, C., Yan X., and Zhou, Z. (2019). “Synthesizing ginsenoside Rh2 in Saccharomyces cerevisiae cell factory at high-efficiency.” Cell Discovery, 5(1): 5.
Zhao, F. L., Bai, P., Nan, W. H., Li, D. S., Zhang, C. B., Lu, C. Z., Qi, H. S., Lu, W. Y., 2019. A modular engineering strategy for high-level production of protopanaxadiol from ethanol by Saccharomyces cerevisiae. Aiche Journal., 65, 866-874.
US20170130233
WO2012/116783
WO2017/004022
   1. An expression cassette encoding a fusion protein, comprising
   a) a nucleotide sequence encoding
      (i) an amino acid sequence shown in SEQ ID NO: 1 or a fragment thereof which directs protein decay, or
      (ii) an amino acid sequence which is at least 60% identical to the amino acid sequence of (i) which directs protein decay; and
   b) a nucleotide sequence encoding a protein of interest, wherein nucleotide sequence a) and b) are fused together in frame.
      2. The expression cassette of item 1, wherein the amino acid sequence as defined in a) is located at the N-terminus, at the C-terminus, or within the protein of interest as defined in b).
      3. The expression cassette of any one the preceding items, further comprising
   c) one or more nucleotide sequence(s) fused to the 5′- and/or 3′-end of the nucleotide sequence a) and/or b);
   d) one or more nucleotide sequence(s) which is/are comprised in the nucleotide sequence a), b) or c) and wherein said nucleotide sequence d) is fused in frame with the nucleotide sequences of a), b) and/or c).
      4. The expression cassette of any one of the preceding items, wherein the nucleotide sequence of a) is shown in SEQ ID NO: 2.
      5. The expression cassette of any one of the preceding items, wherein the level of the fusion protein gradually reduces when expressed in a cell in comparison to a cell which expresses the protein of interest.
      6. The expression cassette of any one of items 3 to 5, wherein said nucleotide sequence of d) comprises at least 3 nucleotides and encodes a heterologous polypeptide, wherein said heterologous polypeptide is a linker, tag and/or cleavable site for a protease.
      7. The expression cassette of any of the preceding items, wherein a constitutive active or inducible expression control sequence is operatively linked with the expression cassette, wherein the inducible expression control sequence is inducible preferably by temperature, light, small molecules or the expression of another protein.
      8. The expression cassette of any one of the preceding items, wherein said nucleotide sequence of b) encodes a polypeptide selected from a group consisting of enzymes, receptors, receptor ligands, antibodies, lipocalins, hormones, inhibitors, membrane proteins, membrane associated proteins, peptidic toxins and peptidic antitoxins.
      9. The expression cassette of item 8, wherein the enzyme is a lanosterol synthase, preferably ERG7 as shown in SEQ ID NO: 3.
      10. The expression cassette of any one of the preceding items further comprising a nucleotide sequence encoding a selection marker which preferably confers resistance against an antibiotic or anti-metabolite.
      11. A vector comprising the expression cassette of any one of items 1 to 10.
      12. A host cell comprising the expression cassette of any one of items 1 to 10 or the vector of item 11.
      13. The host cell of item 12, wherein the protein of interest comprised by the expression cassette is a lanosterol synthase, preferably Erg7p as shown in SEQ ID NO: 3.
      14. The host cell of item 13, wherein the lanosterol synthase comprised by the expression cassette is encoded by the nucleotide sequence as shown in SEQ ID NO. 4.
      15. The host cell of any one of items 12 to 14, which is bacterial host cell, a mammalian host cell, or a fungal host cell, preferably a yeast host cell.
      16. The host cell of any one of items 13 to 15, which further does not express one or more sterol acyltransferases, preferably:
   (i) Are1p as shown in SEQ ID NO: 15 and/or
   (ii) Are2p as shown in SEQ ID NO: 16.
      17. The host cell of any one of items 13 to 16, which further expresses one or more of the following proteins:
   (i) a truncated HMG-COA reductase;
   (ii) an oxidosqualene cyclase;
   (iii) a cytochrome P450 monooxygenase;
   (iv) a cytochrome P450 reductase;
   (v) a sterol acyltransferase.
      18. A method for the production of a triterpenoid, comprising
   (a) culturing a host cell of any one of items 13 to 16 under conditions which allow the production of a triterpenoid; and
   (b) harvesting the triterpenoid produced by said host cell.

1. An expression cassette encoding a fusion protein, comprising
a) a nucleotide sequence encoding
(i) an amino acid sequence shown in SEQ ID NO: 1 or a fragment thereof which directs protein decay, or
(ii) an amino acid sequence which is at least 60% identical to the amino acid sequence of (i) which directs protein decay; and
b) a nucleotide sequence encoding a protein of interest,
wherein nucleotide sequence a) and b) are fused together in frame and wherein the fragment is at least 18 amino acids long.

2. The expression cassette of claim 1, wherein the amino acid sequence as defined in a) is located at the N-terminus, at the C-terminus, or within the protein of interest as defined in b).

3. The expression cassette of claim 1, further comprising
c) one or more nucleotide sequence(s) fused to the 5′- and/or 3′-end of the nucleotide sequence a) and/or b);
d) one or more nucleotide sequence(s) which is/are comprised in the nucleotide sequence a), b) or c) and wherein said nucleotide sequence d) is fused in frame with the nucleotide sequences of a), b) and/or c).

4. The expression cassette of claim 1, wherein the nucleotide sequence of a) is shown in SEQ ID NO: 2.

5. The expression cassette of claim 1, wherein the level of the fusion protein gradually reduces when expressed in a cell in comparison to a cell which expresses the protein of interest.

6. The expression cassette of claim 3, wherein said nucleotide sequence of d) comprises at least 3 nucleotides and encodes a heterologous polypeptide, wherein said heterologous polypeptide is a linker, tag and/or cleavable site for a protease.

7. The expression cassette of claim 1, wherein a constitutive active or inducible expression control sequence is operatively linked with the expression cassette, wherein the inducible expression control sequence is inducible preferably by temperature, light, small molecules or the expression of another protein.

8. The expression cassette of claim 1, wherein said nucleotide sequence of b) encodes a polypeptide selected from a group consisting of enzymes, receptors, receptor ligands, antibodies, lipocalins, hormones, inhibitors, membrane proteins, membrane associated proteins, peptidic toxins and peptidic antitoxins.

9. The expression cassette of claim 8, wherein the enzyme is a lanosterol synthase, preferably ERG7 as shown in SEQ ID NO: 3.

10. The expression cassette of claim 1, further comprising a nucleotide sequence encoding a selection marker which preferably confers resistance against an antibiotic or anti-metabolite.

11. A vector comprising the expression cassette of claim 1.

12. A host cell comprising the expression cassette claim 1.

13. The host cell of claim 12, wherein the protein of interest comprised by the expression cassette is a lanosterol synthase, preferably Erg7p as shown in SEQ ID NO: 3.

14. The host cell of claim 13, wherein the lanosterol synthase comprised by the expression cassette is encoded by the nucleotide sequence as shown in SEQ ID NO. 4.

15. The host cell of claim 12, which is a bacterial host cell, a mammalian host cell, or a fungal host cell.

16. The host cell claim 13, which further does not express one or more sterol acyltransferases, preferably:
(i) Are1p as shown in SEQ ID NO: 15 and/or
(ii) Are2p as shown in SEQ ID NO: 16.

17. The host cell of claim 13, which further expresses one or more of the following proteins:
(i) a truncated HMG-COA reductase;
(ii) an oxidosqualene cyclase;
(iii) a cytochrome P450 monooxygenase;
(iv) a cytochrome P450 reductase;
(v) a sterol acyltransferase.

18. A method for the production of a triterpenoid, comprising
(a) culturing a host cell of claim 13 under conditions which allow the production of a triterpenoid; and
(b) harvesting the triterpenoid produced by said host cell.

19. A host cell comprising the vector of claim 11.

20. The host cell of claim 12, which is a yeast host cell.


SEQUENCE LISTING (46 sequences)

SEQ ID NO: 1 - protein, 31 aa, synthetic construct
  REGION 1..31: note = synthetic decay sequence
LRLEQVLVLVLEQFCLKVKNYSLVLSQFWLN

SEQ ID NO: 2 - DNA, 93 bp, synthetic construct
  misc_feature 1..93: note = synthetic decay sequence
  source 1..93: mol_type = other DNA
cgcttggagcaggtgctggtgctggtgctggagcaattctgtctaaaggtgaagaattattcactggtgttgtcccaattttggttgaattag

SEQ ID NO: 3 - protein, 731 aa, Saccharomyces cerevisiae
MTEFYSDTIGLPKTDPRLWRLRTDELGRESWEYLTPQQAANDPPSTFTQWLLQDPKFPQPHPERNKHSPDFSAFDACHNGASFFKLLQEPDSGIFPCQYKGPMFMTIGYVAVNYIAGIEIPEHERIELIRYIVNTAHPVDGGWGLHSVDKSTVFGTVLNYVILRLLGLPKDHPVCAKARSTLLRLGGAIGSPHWGKIWLSALNLYKWEGVNPAPPETWLLPYSLPMHPGRWWVHTRGVYIPVSYLSLVKFSCPMTPLLEELRNEIYTKPFDKINFSKNRNTVCGVDLYYPHSTTLNIANSLVVFYEKYLRNRFIYSLSKKKVYDLIKTELQNTDSLCIAPVNQAFCALVTLIEEGVDSEAFQRLQYRFKDALFHGPQGMTIMGTNGVQTWDCAFAIQYFFVAGLAERPEFYNTIVSAYKFLCHAQFDTECVPGSYRDKRKGAWGFSTKTQGYTVADCTAEAIKAIIMVKNSPVFSEVHHMISSERLFEGIDVLLNLQNIGSFEYGSFATYEKIKAPLAMETLNPAEVFGNIMVEYPYVECTDSSVLGLTYFHKYFDYRKEEIRTRIRIAIEFIKKSQLPDGSWYGSWGICFTYAGMFALEALHTVGETYENSSTVRKGCDFLVSKQMKDGGWGESMKSSELHSYVDSEKSLVVQTAWALIALLFAEYPNKEVIDRGIDLLKNRQEESGEWKFESVEGVFNHSCAIEYPSYRFLFPIKALGMYSRAYETHTL

SEQ ID NO: 4 - DNA, 2196 bp, Saccharomyces cerevisiae
  source 1..2196: mol_type = genomic DNA
atgacagaattttattctgacacaatcggtctaccaaagacagatccacgtctttggagactgagaactgatgagctaggccgagaaagctgggaatatttaacccctcagcaagccgcaaacgacccaccatccactttcacgcagtggcttcttcaagatcccaaatttcctcaacctcatccagaaagaaataagcattcaccagatttttcagccttcgatgcgtgtcataatggtgcatcttttttcaaactgcttcaagagcctgactcaggtatttttccgtgtcaatataaaggacccatgttcatgacaatcggttacgtagccgtaaactatatcgccggtattgaaattcctgagcatgagagaatagaattaattagatacatcgtcaatacagcacatccggttgatggtggctggggtctacattctgttgacaaatccaccgtgtttggtacagtattgaactatgtaatcttacgtttattgggtctacccaaggaccacccggtttgcgccaaggcaagaagcacattgttaaggttaggcggtgctattggatcccctcactggggaaaaatttggctaagtgcactaaacttgtataaatgggaaggtgtgaaccctgcccctcctgaaacttggttacttccatattcactgcccatgcatccggggagatggtgggttcatactagaggtgtttacattccggtcagttacctgtcattggtcaaattttcttgcccaatgactcctcttcttgaagaactgaggaatgaaatttacactaaaccgtttgacaagattaacttctccaagaacaggaataccgtatgtggagtagacctatattacccccattctactactttgaatattgcgaacagccttgtagtattttacgaaaaatacctaagaaaccggttcatttactctctatccaagaagaaggtttatgatctaatcaaaacggagttacagaatactgattccttgtgtatagcacctgttaaccaggcgttttgcgcacttgtcactcttattgaagaaggggtagactcggaagcgttccagcgtctccaatataggttcaaggatgcattgttccatggtccacagggtatgaccattatgggaacaaatggtgtgcaaacctgggattgtgcgtttgccattcaatactttttcgtcgcaggcctcgcagaaagacctgaattctataacacaattgtctctgcctataaattcttgtgtcatgctcaatttgacaccgagtgcgttccaggtagttatagggataagagaaagggggcttggggcttctcaacaaaaacacagggctatacagtggcagattgcactgcagaagcaattaaagccatcatcatggtgaaaaactctcccgtctttagtgaagtacaccatatgattagcagtgaacgtttatttgaaggcattgatgtgttattgaacctacaaaacatcggatcttttgaatatggttcctttgcaacctatgaaaaaatcaaggccccactagcaatggaaaccttgaatcctgctgaagtttttggtaacataatggtagaatacccatacgtggaatgtactgattcatccgttctggggttgacatattttcacaagtacttcgactataggaaagaggaaatacgtacacgcatcagaatcgccatcgaattcataaaaaaatctcaattaccagatggaagttggtatggaagctggggtatttgttttacatatgccggtatgtttgcattggaggcattacacaccgtgggggagacctatgagaattcctcaacggtaagaaaaggttgcgacttcttggtcagtaaacagatgaaggatggcggttggggggaatcaatgaagtccagtgaattacatagttatgtggatagtgaaaaatcgctagtcgttcaaaccgcatgggcgctaattgcacttcttttcgctgaatatcctaataaagaagtcatcgaccgcggtattgaccttttaaaaaatagacaagaagaatccggggaatggaaatttgaaagtgtagaaggtgttttcaaccactcttgtgcaattgaatacccaagttatcgattcttattccctattaaggcattaggtatgtacagcagggcatatgaaacacatacgctttaa

SEQ ID NO: 5 - protein, 527 aa, Saccharomyces cerevisiae
MTADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 6 - protein, 758 aa, Olea europaea
MWKLKIADGTGPWLTTTNNHIGRQHWEFDPEAGTPDERVEVERLREEFKKNRFRTKQSADLLMRMQLVKENQRVQIPPAIKIKETEGITEEAVITTLRRAISFYSTIQAHDGHWPAESAGPLFFLPPLVLALYVTGAINVVLSREHQKEITRYIYNHQNEDGGWGIHIEGHSTMFGSVLSYITLRLLGEGQEDGEDKAVARGRKWILDHGGAVGIPSWGKFWLTVLGVYEWDGCNPMPPEFWLLPNFSPIHPGKMLCYCRLVYMPMSYLYGKRFVGPITGLVLSLRQEIYTEPYHGINWNRARNTCAKEDLYYPHPLAQDMLWGFLHHFAEPVLTRWPFSKLREKALKVAMEHVHYEDMNSRYLCIGCVEKVLCLIACWVEDPNSEAYKRHIARIPDYFWVAEDGLKMQSFGCQMWDAAFAIQAILSSNLAEEYGPTLMKAHNFVKASQVQENPSGDFNEMYRHTSKGAWTFSMQDHGWQVSDCTAEGLKAALLFSQMPIELVGAEIETGHLYDAVNVILTLQSASGGFPAWEPQKAYRWLEKLNPTEFFEDVLIERDYVECTSSAVQALKLFKQLHPGHRRKEIASCISKAIQYIEATQNPDGSWDGSWGICFTYGTWFAVEGLVACGKNYHNSPTLRRACEFLLSKQLPDGGWSESYLSSSNKVYTNLEGNRSNLVQTSWALLSLIKAGQVEIDPGPIHRGIKLLVNSQMEDGDFPQEEITGAFMKNCTLNYSSYRNIFPIWALGEYRRRILHAQT

SEQ ID NO: 7 - protein, 479 aa, Medicago truncatula
MEPNFYLSLLLLFVTFISLSLFFIFYKQKSPLNLPPGKMGYPIIGESLEFLSTGWKGHPEKFIFDRMRKYSSELFKTSIVGESTVVCCGAASNKFLFSNENKLVTAWWPDSVNKIFPTTSLDSNLKEESIKMRKLLPQFFKPEALQRYVGVMDVIAQRHFVTHWDNKNEITVYPLAKRYTFLLACRLFMSVEDENHVAKFSDPFQLIAAGIISLPIDLPGTPFNKAIKASNFIRKELIKIIKQRRVDLAEGTASPTQDILSHMLLTSDENGKSMNELNIADKILGLLIGGHDTASVACTFLVKYLGELPHIYDKVYQEQMEIAKSKPAGELLNWDDLKKMKYSWNVACEVMRLSPPLQGGFREAITDFMFNGFSIPKGWKLYWSANSTHKNAECFPMPEKFDPTRFEGNGPAPYTFVPFGGGPRMCPGKEYARLEILVFMHNLVKRFKWEKVIPDEKIIVDPFPIPAKDLPIRLYPHKA

SEQ ID NO: 8 - protein, 480 aa, Vitis vinifera
MEVFFLSLLLIFVLSVSIGLHLLFYKHRSHFTGPNLPPGKIGWPMVGESLEFLSTGWKGHPEKFIFDRISKYSSEVFKTSLLGEPAAVFAGAAGNKFLFSNENKLVHAWWPSSVDKVFPSSTQTSSKEEAKKMRKLLPQFFKPEALQRYIGIMDHIAQRHFADSWDNRDEVIVFPLAKRFTFWLACRLFMSIEDPAHVAKFEKPFHVLASGLITVPIDLPGTPFHRAIKASNFIRKELRAIIKQRKIDLAEGKASQNQDILSHMLLATDEDGCHMNEMEIADKILGLLIGGHDTASAAITFLIKYMAELPHIYEKVYEEQMEIANSKAPGELLNWDDVQNMRYSWNVACEVMRLAPPLQGAFREAITDFVFNGFSIPKGWKLYWSANSTHKSPECFPQPENFDPTRFEGNGPAPYTFVPFGGGPRMCPGKEYARLEILVFMHNVVKRFKWDKLLPDEKIIVDPMPMPAKGLPVRLHPHKP

SEQ ID NO: 9 - protein, 480 aa, Vitis vinifera
MEVFFLSLLLISVLSVSIRLYLLLYKHRSHFTGPNLPPGKIGWPMVGESLEFLSTGWKGHPEKFIFDRISKYSSEVFKTSLLGEPAAVFAGAAGNKFLFSNENKLVHAWWPSSVDKVFPSSTQTSSKEEAKKMRKLLPQFLKPEALQRYTGIMDHIAQRHFADSWDNRDEVIVFPLAKRFTFWLACRLFMSIEDPAHVAKFEKPFHVLASGLITIPIDLPGTPFHRAIKASNFIRKELRAIIKQRKIDLAESKASKTQDILSHMLLATDEDGCHMNEMSIADKILGLLIGGHDTASSAITFLVKYMAELPHIYEKVYKEQMEIANSKAPGELLNWDDVQKMRYSWNVACEVMRLAPPLQGAFREAITDFVFNGFSIPKGWKLYWSANSTHKSLECFPQPEKFDPTRFEGAGPAPYTFVPFGGGPRMCPGKEYARLEILIFMHNLVKRFKWDKLLPDEKIIVDPMPMPAKGLPVRLHPHKP

SEQ ID NO: 10 - protein, 486 aa, Panax ginseng
MAAAMVLFFSLSLLLLPLLLLFAYFSYTKRIPQKENDSKAPLPPGQTGWPLIGETLNYLSCVKSGVSENFVKYRKEKYSPKVFRTSLLGEPMAILCGPEGNKFLYSTEKKLVQVWFPSSVEKMFPRSHGESNADNFSKVRGKMMFLLKVDGMKKYVGLMDRVMKQFLETDWNRQQQINVHNTVKKYTVTMSCRVFMSIDDEEQVTRLGSSIQNIEAGLLAVPINIPGTAMNRAIKTVKLLTREVEAVIKQRKVDLLENKQASQPQDLLSHLLLTANQDGQFLSESDIASHLIGLMQGGYTTLNGTITFVLNYLAEFPDVYNQVLKEQVEIANSKHPKELLNWEDLRKMKYSWNVAQEVLRIIPPGVGTFREAITDFTYAGYLIPKGWKMHLIPHDTHKNPTYFPSPEKFDPTRFEGNGPAPYTFTPFGGGPRMCPGIEYARLVILIFMHNVVTNFRWEKLIPNEKILTDPIPRFAHGLPIHLHPHN

SEQ ID NO: 11 - protein, 692 aa, Arabidopsis thaliana [sequence omitted]

SEQ ID NO: 12 - protein, 711 aa, Arabidopsis thaliana
MSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDINMANGNGYTVFDAQHPYKANVAVKRELHTPESDRSCIHLEFDIAGSGLTYETGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSENCSSAPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 13 - protein, 706 aa, Lotus japonicus
MEESSSMKISPLDLMSAMIKGTLDPSNVSSTSGAGSVFLENREFVMVLTTSIAVLIGCVVVFIWRRSTGNKAKSIEPPKRVVEKLSDEAEVDDGTRKVTIFFGTQTGTAEGFAKAIAEEAKVRYEKAKFKIVDMDDYAQDDDEYEEKLKKETLALFFLATYGDGEPTDNAARFYKWFLEGDEKEEGWLRNLEYAVFGLGNRQYEHFNKVAIEVDDKLADFGGKRLVKVGLGDDDQCIEDDFTAWKEELWPALDELLRGDDDTTVSTPYTAAVLEYRVVIHDPLDASVDEKKWHNVNGHAIVDAQHPVRSNVAVRKELHTPVSDRSCTHLEFDISGTGVAYETGDHVGVYCENLSETVEEAVRLLGLSPDTYFSVHTDDEDGKPLSGSSLPPTFPPCTLRTAIARYADVLSSPKKSVLLALAAHASNPSEADRLRHLASPAGKDEYSEWVIASQRSLLEVMAEFPSAKPPIGVFFAAIAPRLQPRFYSISSSPRMAPSRIHVTCALVNDKMPTGRIHRGVCSTWMKNSVPLEKSQDCSWAPIFVRQSNFKLPADNKVPIIMIGPGTGLAPFRGFLQERLALKEDGAELGPSVLFFGCRNRQMDYIYEDELNHFVNSGALSELIVAFSREGPTKEYVQHKMMEKASDIWNMISQGAYIYVCGDAKGMARDVHRTLHTILQEQGSLDSSKAEGMVKNLQLNGRYLRDVW

SEQ ID NO: 14 - protein, 714 aa, Catharanthus roseus
MDSSSEKLSPFELMSAILKGAKLDGSNSSDSGVAVSPAVMAMLLENKELVMILTTSVAVLIGCVVVLIWRRSSGSGKKVVEPPKLIVPKSVVEPEEIDEGKKKFTIFFGTQTGTAEGFAKALAEEAKARYEKAVIKVIDIDDYAADDEEYEEKFRKETLAFFILATYGDGEPTDNAARFYKWFVEGNDRGDWLKNLQYGVFGLGNRQYEHFNKIAKVVDEKVAEQGGKRIVPLVLGDDDQCIEDDFAAWRENVWPELDNLLRDEDDTTVSTTYTAAIPEYRVVFPDKSDSLISEANGHANGYANGNTVYDAQHPCRSNVAVRKELHTPASDRSCTHLDFDIAGTGLSYGTGDHVGVYCDNLSETVEEAERLLNLPPETYFSLHADKEDGTPLAGSSLPPPFPPCTLRTALTRYADLLNTPKKSALLALAAYASDPNEADRLKYLASPAGKDEYAQSLVANQRSLLEVMAEFPSAKPPLGVFFAAIAPRLQPRFYSISSSPRMAPSRIHVTCALVYEKTPGGRIHKGVCSTWMKNAIPLEESRDCSWAPIFVRQSNFKLPADPKVPVIMIGPGTGLAPFRGFLQERLALKEEGAELGTAVFFFGCRNRKMDYIYEDELNHFLEIGALSELLVAFSREGPTKQYVQHKMAEKASDIWRMISDGAYVYVCGDAKGMARDVHRTLHTIAQEQGSMDSTQAEGFVKNLQMTGRYLRDVW

SEQ ID NO: 15 - protein, 604 aa, Saccharomyces cerevisiae
MTETKDLLQDEEFLKIRRLNSAEPNKRHSVTYDNVILPQESMEVSPRSSTTSLVEPVESSEAERVAGKQEQEEEYPVDAHMQKYLSHLKSKSRSRFHRKDASKYVSFFGDVSFDPRPTLLDSAINVPFQTTFKGPVLEKQLKNLQLTKTKAKATVKATVKTTEKTDKADAPPGEKLESNFSGIYVFAWMFLGWIAIRCCTDYYASYGSAWNKLEIVQYMTTDLFTIAMLDLAMFLCTFFVVFVHWLVKKRIINWKWTGFVAVSIFELAFIPVTFPIYVYYFDFNWVTRIFLFLHSVVFVMKSHSFAFYNGYLWDIKQELEYSSKQLQKYKESLSPETREILQKSCDFCLFELNYQTKDNDFPNNISCSNFFMFCLFPVLVYQINYPRTSRIRWRYVLEKVCAIIGTIFLMMVTAQFFMHPVAMRCIQFHNTPTFGGWIPATQEWFHLLFDMIPGFTVLYMLTFYMIWDALLNCVAELTRFADRYFYGDWWNCVSFEEFSRIWNVPVHKFLLRHVYHSSMGALHLSKSQATLFTFFLSAVFHEMAMFAIFRRVRGYLFMFQLSQFVWTALSNTKFLRARPQLSNVVFSFGVCSGPSIIMTLYLTL

SEQ ID NO: 16 - protein, 642 aa, Saccharomyces cerevisiae
MDKKKDLLENEQFLRIQKLNAADAGKRQSITVDDEGELYGLDTSGNSPANEHTATTITQNHSVVASNGDVAFIPGTATEGNTEIVTEEVIETDDNMFKTHVKTLSSKEKARYRQGSSNFISYFDDMSFEHRPSILDGSVNEPFKTKFVGPTLEKEIRRREKELMAMRKNLHHRKSSPDAVDSVGKNDGAAPTTVPTAATSETVVTVETTIISSNFSGLYVAFWMAIAFGAVKALIDYYYQHNGSFKDSEILKFMTTNLFTVASVDLLMYLSTYFVVGIQYLCKWGVLKWGTTGWIFTSIYEFLFVIFYMYLTENILKLHWLSKIFLFLHSLVLLMKMHSFAFYNGYLWGIKEELQFSKSALAKYKDSINDPKVIGALEKSCEFCSFELSSQSLSDQTQKFPNNISAKSFFWFTMFPTLIYQIEYPRTKEIRWSYVLEKICAIFGTIFLMMIDAQILMYPVAMRALAVRNSEWTGILDRLLKWVGLLVDIVPGFIVMYILDFYLIWDAILNCVAELTRFGDRYFYGDWWNCVSWADFSRIWNIPVHKFLLRHVYHSSMSSFKLNKSQATLMTFFLSSVVHELAMYVIFKKLRFYLFFFQMLQMPLVALTNTKFMRNRTIIGNVIFWLGICMGPSVMCTLYLTF

SEQ ID NO: 17 - protein, 761 aa, Artemisia annua
MWRLKIAEGRNDPYLYSTNNFVGRQIWEFDPNYGTPEERAEVEQARVDFWNHRHEVKPSSDVLWRMQFLREKGFEQTIPQVKIEDGEEISYEKATTTLRRSVNFFAALQADDGHWPAENAGPLYFMQPLVICLYITGHLNTVFPAEYRKEILRYIYCHQNEDGGWGFHIEGHSTMFCTTLSYICMRLLGEGRDGGLDGACTKARKWILDHGSVTTIPSWGKTWLSILGVCEWAGTNPMPPEFWILPSFLPMYPAKMWCYCRLVYMPMSYLYGKRFVGPITPLILQLRDELYAQPYDEIKWRSIRHLCAKEDLYYPHPLLQDLMWDSLYVFTEPVLNHWPFNKLREKALQTTMKHIHYEDENSRYITIGSVEKALCMLACWVEDPNGVCFKKHIARIPDYLWVAEDGMKMQSFGSQEWDAGFAIQALMATDLTDEIGSTLMKGHEFIKASQVKDNPSGDFKSMHRHISKGSWTFSDQDHGWQVSDCTAEALKCCLLFATMPPEIVGEKMKPEQLNDAVNVILSLQSKNGGLAAWEPAGSSEWLEILNPTEFFADIVIEHEYVECTSSAIQALVMFKKKYPGHRKKEIENFLLGSSGYLEKIQMEDGSWYGNWGVCFTYGTWFALGGLSAVGKTYDNCPAIRKAVKFLLETQLEDGGWGESYKSCPEKKYIPLEGGRSNLVHTAWAMMGLIHSRQAERDATPLHRAAKLLINSQLETGDFPQQEIAGVFMKNCMLHYALYRNIYPMWALADYRKQVLPQLKGT

SEQ ID NO: 18 - protein, 769 aa, Panax ginseng
MWKLKVAQGNDPYLYSTNNFVGRQYWEFQPDAGTPEEREEVEKARKDYVNNKKLHGIHPCSDMLMRRQLIKESGIDLLSIPPLRLDENEQVNYDAVTTAVKKALRLNRAIQAHDGHWPAENAGSLLYTPPLIIALYISGTIDTILTKQHKKELIRFVYNHQNEDGGWGSYIEGHSTMIGSVLSYVMLRLLGEGLAESDDGNGAVERGRKWILDHGGAAGIPSWGKTYLAVLGVYEWEGCNPLPPEFWLFPSSFPFHPAKMWIYCRCTYMPMSYLYGKRYHGPITDLVLSLRQEIYNIPYEQIKWNQQRHNCCKEDLYYPHTLVQDLVWDGLHYFSEPFLKRWPFNKLRKRGLKRVVELMRYGATETRFITTGNGEKALQIMSWWAEDPNGDEFKHHLARIPDFLWIAEDGMTVQSFGSQLWDCILATQAIIATNMVEEYGDSLKKAHFFIKESQIKENPRGDFLKMCRQFTKGAWTFSDQDHGCVVSDCTAEALKCLLLLSQMPQDIVGEKPEVERLYEAVNVLLYLQSRVSGGFAVWEPPVPKPYLEMLNPSEIFADIVVEREHIECTASVIKGLMAFKCLHPGHRQKEIEDSVAKAIRYLERNQMPDGSWYGFWGICFLYGTFFTLSGFASAGRTYDNSEAVRKGVKFFLSTQNEEGGWGESLESCPSEKFTPLKGNRTNLVQTSWAMLGLMFGGQAERDPTPLHRAAKLLINAQMDNGDFPQQEITGVYCKNSMLHYAEYRNIFPLWALGEYRKRVWLPKHQQLKI

SEQ ID NO: 19 - protein, 759 aa, synthetic construct
  REGION 1..759: note = yeast modified
MTEFYSDTIGLPKTDPRLWRLRTDELGRESWEYLTPQQAANDPPSTFTQWLLQDPKFPQPHPERNKHSPDFSAFDACHNGASFFKLLQEPDSGIFPCQYKGPMFMTIGYVAVNYIAGIEIPEHERIELIRYIVNTAHPVDGGWGLHSVDKSTVFGTVLNYVILRLLGLPKDHPVCAKARSTLLRLGGAIGSPHWGKIWLSALNLYKWEGVNPAPPETWLLPYSLPMHPGRWWVHTRGVYIPVSYLSLVKFSCPMTPLLEELRNEIYTKPFDKINFSKNRNTVCGVDLYYPHSTTLNIANSLVVFYEKYLRNRFIYSLSKKKVYDLIKTELQNTDSLCIAPVNQAFCALVTLIEEGVDSEAFQRLQYRFKDALFHGPQGMTIMGTNGVQTWDCAFAIQYFFVAGLAERPEFYNTIVSAYKFLCHAQFDTECVPGSYRDKRKGAWGFSTKTQGYTVADCTAEAIKAIIMVKNSPVFSEVHHMISSERLFEGIDVLLNLQNIGSFEYGSFATYEKIKAPLAMETLNPAEVFGNIMVEYPYVECTDSSVLGLTYFHKYFDYRKEEIRTRIRIAIEFIKKSQLPDGSWYGSWGICFTYAGMFALEALHTVGETYENSSTVRKGCDFLVSKQMKDGGWGESMKSSELHSYVDSEKSLVVQTAWALIALLFAEYPNKEVIDRGIDLLKNRQEESGEWKFESVEGVFNHSCAIEYPSYRFLFPIKALGMYSRAYETLRLEQVLVLVLEQFCLKVKNYSLVLSQFWLN

SEQ ID NO: 20 - protein, 759 aa, synthetic construct; identical to SEQ ID NO: 19
  REGION 1..759: note = yeast modified

SEQ ID NO: 21 - DNA, 1584 bp, Saccharomyces cerevisiae
  source 1..1584: mol_type = genomic DNA
atgactgcagaccaattggtgaagactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaagggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgtcgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccacatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctga

SEQ ID NO: 22 - DNA, 2277 bp, Olea europaea
  source 1..2277: mol_type = genomic DNA
atgtggaagttaaaaatagcagacggtacaggtccttggttgactactacaaataatcatatcggtagacaacactgggaattcgacccagaagctggtacacctgatgaaagagtagaagttgaaagattgagagaagaattcaagaaaaatagattcagaaccaagcaatcagctgacttgttgatgagaatgcaattagttaaggaaaaccaaagagtccaaataccacctgctattaaaattaaagaaacagaaggtataaccgaagaagccgttataactacattgagaagagctatctcattttattccactattcaagcacatgatggtcactggccagctgaaagtgcaggtcctttgtttttcttaccacctttggttttagcattgtacgtcactggtgccattaatgttgtcttatctagagaacatcaaaaggaaataacaagatacatatacaaccaccaaaacgaagatggtggttggggtatccatattgaaggtcactctacaatgttcggtagtgttttgtcttacatcaccttgagattgttgggtgaaggtcaagaagatggtgaagacaaagcagttgccagaggtagaaagtggatattggatcatggtggtgctgtcggtatcccatcttggggtaaattttggttgacagttttaggtgtctacgaatgggacggttgtaatccaatgccacctgaattttggttgttacctaacttctcaccaatccaccctggtaaaatgttgtgttactgcagattagtttacatgccaatgagttatttgtacggtaaaagattcgtaggtcctataactggtttagttttgtctttgagacaagaaatctatacagaaccatatcatggtattaattggaacagagccagaaacacttgtgctaaggaagatttgtactacccacaccctttagcccaagacatgttgtggggtttcttgcatcacttcgctgaaccagttttgacaagatggcctttctcaaagttgagagaaaaggctttgaaggtagcaatggaacatgttcactacgaagatatgaattccagatacttatgtattggttgcgtagaaaaggttttgtgtttgatcgcttgctgggtcgaagacccaaactcagaagcttataagagacatatagcaagaatccctgattacttttgggttgcagaagacggtttgaaaatgcaatcctttggttgtcaaatgtgggatgctgcattcgccattcaagctatcttgtcttcaaatttggctgaagaatatggtccaactttaatgaaagcccataactttgtcaaggctagtcaagtacaagaaaatccttctggtgacttcaacgaaatgtacagacatacttcaaagggtgcatggacattttccatgcaagatcacggttggcaagtttcagactgcacagccgaaggtttgaaagccgctttgttattctcccaaatgccaatagaattggttggtgcagaaatcgaaaccggtcatttgtatgatgccgtcaacgtaatattgactttgcaatcagcatccggtggttttccagcctgggaacctcaaaaggcttacagatggttggaaaagttgaacccaaccgaatttttcgaagatgtcttgattgaaagagactacgtagaatgtacttccagtgcagttcaagccttgaaattattcaagcaattacatccaggtcacagaagaaaagaaatcgcatcatgtatttccaaggccatccaatatattgaagctacccaaaatcctgatggtagttgggacggttcttggggtatttgctttacctatggtacttggttcgcagtcgaaggtttggtagcctgtggtaaaaattaccataacagtccaacattgagaagagcttgcgaattcttgttgtctaagcaattgcctgatggtggttggagtgaatcttatttgtcttcttctaataaggtctacacaaacttggaaggtaatagatctaacttagtacaaacctcatgggctttgttatccttgattaaagcaggtcaagtagaaatagatccaggtcctatccatagaggtatcaagttgttagttaactctcaaatggaagatggtgactttccacaagaagaaataaccggtgctttcatgaaaaattgtactttgaactatagttcttacagaaacatctttcctatctgggctttgggtgaatacagaagaagaatattgcatgcacaaacttaa

SEQ ID NO: 23 - DNA, 1440 bp, Medicago truncatula
  source 1..1440: mol_type = genomic DNA
atggagcctaatttctatctctcccttctccttctctttgtctctttcatatctctctctctattttttatattctacaaacagaaatctccattaaatttgccacctggtaaaatgggttacccaatcataggtgaaagccttgagttcttatcaacaggatggaaaggacatcctgaaaaattcattttcgaccgtatgcgtaaatattcctcagaactctttaaaacatcaatcgtaggagaatctacggtggtttgttgcggagcagcaagtaacaagtttttgttttcaaacgagaataaacttgtgactgcatggtggccggatagtgtaaacaaaatcttccctactacttctcttgactctaacttgaaggaagaatccatcaagatgagaaaattgcttccacaattctttaaacccgaagctctacaacgttatgttggtgtcatggatgttattgctcaaagacattttgttactcattgggataataaaaatgaaatcaccgtctaccccttggccaagaggtacacctttttgttagcttgtcggttgttcatgagcgttgaagacgagaatcatgtagcaaaatttagtgatccatttcagttaattgcagccggaatcatatctctaccaattgatttgccaggaacaccattcaacaaagctataaaggcctcaaactttataagaaaggagttgattaagatcataaagcaaaggaggatagatttggcagaagggacagcatcaccaacacaagatatattgtctcacatgttgttgacaagtgatgaaaatggaaagagtatgaatgaacttaatattgctgataagattcttggccttttgatcggaggacatgacactgctagcgtcgcatgcactttccttgtcaaatatctcggcgagctacctcacatttatgataaagtctatcaagagcaaatggaaattgcaaaatcgaaaccagcaggagaattgttgaattgggatgacctgaagaaaatgaaatactcttggaacgtagcttgtgaagtaatgagactttcccctccactccaaggaggtttcagggaagccatcactgactttatgttcaatggattctcaattcctaagggatggaagctttattggagtgcaaattcaacacataagaacgcagaatgttttcccatgccagagaaatttgacccaacaagatttgaaggaaatggaccagctccttatacttttgtgccctttggtggaggaccaaggatgtgtcctggaaaagagtatgcaagattagaaatacttgttttcatgcacaatttggtgaaaaggtttaagtgggaaaaggtgattccagatgagaagattattgttgatccattccccatccctgcaaaggatcttccaattcgcctttatccacacaaagcttaa

SEQ ID NO: 24 - DNA, 1443 bp, Vitis vinifera
  source 1..1443: mol_type = genomic DNA
atggaagtattctttttgtcattgttgttgattttcgtattgtcagtttctattggtttacatttgttgttctataagcacagatcccattttacaggtcctaacttgccacctggtaaaattggttggccaatggttggtgaatccttggaattcttaagtaccggttggaagggtcaccctgaaaagtttattttcgatagaatctccaagtattcttcagaagtttttaaaacttctttgttgggtgaaccagctgcagtattcgcaggtgccgctggtaataagttcttgttctccaacgaaaacaaattagttcatgcctggtggccttccagtgtcgataaggtatttccatcttcaactcaaacatccagtaaagaagaagccaaaaagatgagaaagttgttgcctcaatttttcaagccagaagctttgcaaagatacataggtatcatggatcatatagcacaaagacactttgccgattcttgggacaacagagatgaagtaattgttttcccattagccaaaagattcactttctggttggcttgtagattgtttatgtctattgaagaccctgctcatgttgcaaaatttgaaaagccattccacgtattggcttcaggtttaatcacagttccaattgatttgccaggtactccatttcatagagccatcaaggcttctaacttcatcagaaaggaattgagagcaattattaagcaaagaaagatcgacttagcagagggtaaagcctctcaaaaccaagatatcttgtcacatatgttgttggctacagatgaagacggttgtcacatgaacgaaatggaaatcgcagacaagatcttgggtttgttgattggtggtcatgataccgcatcagcagccatcacatttttgattaaatacatggccgaattacctcacatctatgaaaaagtctacgaagaacaaatggaaatagctaattccaaagcaccaggtgaattgttaaattgggatgacgttcaaaacatgagatacagttggaatgttgcttgcgaagtcatgagattggcaccacctttacaaggtgcctttagagaagctataaccgactttgttttcaacggtttctctatcccaaaaggttggaagttgtactggtctgctaactcaactcataagtcacctgaatgttttccacaacctgaaaactttgatccaactagattcgagggtaatggtccagctccttatacatttgtaccattcggtggtggtcctagaatgtgtccaggtaaagaatacgcaagattggaaattttggtttttatgcacaacgttgtcaagagattcaagtgggacaagttgttgcctgatgaaaagattatagttgatccaatgcctatgccagctaagggtttgccagtcagattacatcctcacaaaccataa

SEQ ID NO: 25 - DNA, 1443 bp, Vitis vinifera
  source 1..1443: mol_type = genomic DNA
atggaggtgttcttcctctccctgctcctcatctctgtgctctcagtctccatcagactttacttgctcttatacaagcatagatcccacttcactggccccaatctccctcctggcaagattggttggccaatggttggtgaaagccttgaattcctctccaccggctggaaaggccacccggaaaaattcatcttcgatcgcatctccaaatactcctctgaagtcttcaagacctccctcctcggagagcctgctgccgtctttgctggcgctgcgggcaacaagtttttgttctccaacgaaaacaaacttgttcatgcatggtggcctagctccgtcgacaaggtcttcccctcctccacccaaacctcatccaaagaggaggccaagaagatgaggaagttgctccctcagttccttaagcctgaagccttgcaacgttacaccggcatcatggatcacattgcacagaggcattttgctgatagctgggacaacagagatgaagtcattgtatttccactggccaagaggttcactttctggctagcttgccgcctgtttatgagcatagaagatcctgcccacgtcgctaaatttgaaaagcccttccacgtcttggcctcaggactcatcaccatcccaattgacctgcctgggacacctttccaccgcgctatcaaggcctccaacttcatcagaaaggagcttagagccatcatcaagcaaaggaagatcgatctggctgagagcaaggcctcaaaaactcaagatatattgtcccacatgcttctggctacagatgaagatggatgccacatgaatgaaatgagtattgctgataaaatcctcggtttgttgattggtggccatgacactgccagttctgccattacattccttgtcaagtacatggctgagctgcctcacatctacgagaaagtctacaaggagcaaatggaaattgccaattcaaaagcaccaggtgaattgctgaactgggatgatgttcaaaagatgagatattcatggaatgttgcctgtgaagtgatgagacttgcacccccactccaaggagctttccgggaagcaatcactgacttcgtgttcaacggtttctccattcctaagggttggaagctgtactggagcgcaaactcaacccacaaaagcctagaatgcttccctcaacccgaaaaatttgaccctacaagatttgaaggagccgggcctgctccttacacattcgttccctttggtggcggacctaggatgtgccctggtaaagagtacgcccgcttggagatacttatcttcatgcacaacttggttaaaaggttcaaatgggataaattgcttcctgatgagaagataatcgttgaccccatgcccatgcctgctaagggacttccagttcgcctccatcctcacaaaccatag

SEQ ID NO: 26 - DNA, 1461 bp, Panax ginseng
  source 1..1461: mol_type = genomic DNA
atggcagcagcaatggtgttgtttttctccctatctcttcttctccttcccctattattattgtttgcctatttttcttatacaaaacgcatcccccagaaagaaaatgattcaaaagctcccctccccccaggtcaaacaggttggcctttgataggcgaaactcttaattatttatcttgtgtcaaaagtggggtttctgaaaattttgtgaaatataggaaggaaaagtattcccccaaagttttcaggacatcacttttaggagaaccgatggcaatcttgtgtgggccggagggcaacaaattcctctactcaacggaaaaaaagctagtccaagtttggttcccgagcagtgttgaaaagatgttccccagatctcatggcgaatccaacgcagacaacttctccaaagtacgcggcaaaatgatgtttctactcaaggtggacgggatgaaaaaatatgttggcctaatggacagggtgatgaaacagtttttagagacagattggaatcgccaacaacagatcaacgttcataacacggttaagaaatacacggtcacgatgtcgtgtcgggtgtttatgagtatcgatgatgaagagcaagttacaagacttggcagctcaattcagaacatagaggccggactccttgccgtgcctataaatataccggggactgctatgaatcgtgccattaagaccgtaaagttgctaactagagaggttgaggcggtgattaagcaaagaaaagtggatcttttggagaataagcaagcgtcccaaccgcaagatttattgtcacacttgctacttacggccaatcaggatggccaatttttgagcgaatcggatattgctagccacttgataggcttgatgcaaggtggctataccaccttaaatggtacaatcaccttcgttctcaactatcttgcagagtttcctgatgtctacaatcaagtccttaaagagcaagtggaaatagcaaactcaaaacacccaaaagagttgcttaattgggaggatttgaggaagatgaagtattcgtggaatgttgctcaagaagtattgagaataataccaccaggagttggaacattcagagaagctattaccgatttcacctatgctggatatttaattccaaagggatggaagatgcatctgattccacatgacacgcacaagaacccaacatattttccaagtccagaaaaattcgatccaaccaggtttgaaggaaatggtccggctccatatacatttactcctttcggaggaggacctcgaatgtgtccgggaattgagtatgcacgtctagtaatactcatttttatgcacaatgtggttacaaacttcagatgggagaagctcatccctaatgaaaaaattctcaccgatcccattccaagatttgcgcatggacttcccattcatctacatccccacaattaa

SEQ ID NO: 27 - DNA, 2564 bp, synthetic construct
  misc_feature 1..2564: note = yeast modified
  source 1..2564: mol_type = other DNA
aaggtaaaataaaagtcaaataagaaaaggagagaaggacccactcagattcaacgccccaccaccgataactcaattaactctcttctcttggcttctgcaacgctgagccaccatcgtcctcttctcttcctcttcgatctctcctgaatcctcaaaccctggagagagagagagagagtgacgggggaagaagatgacttctgctttgtatgcttccgatttgtttaagcagctcaagtcaattatggggacagattcgttatccgacgatgttgtacttgtgattgcaacgacgtctttggcactagtagctggatttgtggtgttgttatggaagaaaacgacggcggatcggagcggggagctgaagcctttgatgatccctaagtctcttatggctaaggacgaggatgatgatttggatttgggatccgggaagactagagtctctatcttcttcggtacgcagactggaacagctgagggatttgctaaggcattatccgaagaaatcaaagcgagatatgaaaaagcagcagtcaaagtcattgacttggatgactatgctgccgatgatgaccagtatgaagagaaattgaagaaggaaactttggcatttttctgtgttgctacttatggagatggagagcctactgacaatgctgccagattttacaaatggtttacggaggaaaatgaacgggatataaagcttcaacaactagcatatggtgtgtttgctcttggtaatcgccaatatgaacattttaataagatcgggatagttcttgatgaagagttatgtaagaaaggtgcaaagcgtcttattgaagtcggtctaggagatgatgatcagagcattgaggatgattttaatgcctggaaagaatcactatggtctgagctagacaagctcctcaaagacgaggatgataaaagtgtggcaactccttatacagctgttattcctgaataccgggtggtgactcatgatcctcggtttacaactcaaaaatcaatggaatcaaatgtggccaatggaaatactactattgacattcatcatccctgcagagttgatgttgctgtgcagaaggagcttcacacacatgaatctgatcggtcttgcattcatctcgagttcgacatatccaggacgggtattacatatgaaacaggtgaccatgtaggtgtatatgctgaaaatcatgttgaaatagttgaagaagctggaaaattgcttggccactctttagatttagtattttccatacatgctgacaaggaagatggctccccattggaaagcgcagtgccgcctcctttccctggtccatgcacacttgggactggtttggcaagatacgcagaccttttgaaccctcctcgaaagtctgcgttagttgccttggcggcctatgccactgaaccaagtgaagccgagaaacttaagcacctgacatcacctgatggaaaggatgagtactcacaatggattgttgcaagtcagagaagtcttttagaggtgatggctgcttttccatctgcaaaacccccactaggtgtattttttgctgcaatagctcctcgtctacaacctcgttactactccatctcatcctcgccaagattggcgccaagtagagttcatgttacatccgcactagtatatggtccaactcctactggtagaatccacaagggtgtgtgttctacgtggatgaagaatgcagttcctgcggagaaaagtcatgaatgtagtggagccccaatctttattcgagcatctaatttcaagttaccatccaacccttcaactccaatcgttatggtgggacctgggactgggctggcaccttttagaggttttctgcaggaaaggatggcactaaaagaagatggagaagaactaggttcatctttgctcttctttgggtgtagaaatcgacagatggactttatatacgaggatgagctcaataattttgttgatcaaggcgtaatatctgagctcatcatggcattctcccgtgaaggagctcagaaggagtatgttcaacataagatgatggagaaggcagcacaagtttgggatctaataaaggaagaaggatatctctatgtatgcggtgatgctaagggcatggcgagggacgtccaccgaactctacacaccattgttcaggagcaggaaggtgtgagttcgtcagaggcagaggctatagttaagaaacttcaaaccgaaggaagatacctcagagatgtctggtgattgatgatatgatttattggtaattctcttctgctttctcaacttttcttggtacgctgtccttaccatttagatatttatttgtttattttccttcgtctcccttcttttagtcaaacatgaatgatttgtcaaagttttctttgtatgaacaaaagtataaactacaaaataaatctgtgagagtgataaaattgagagaagatacccaagatttcatcatcatgctttctactgtcgatagtggatcatcattgtgaatcagtgacaccaaacaagaacaatttat

SEQ ID NO: 28 - DNA, 2133 bp, Arabidopsis thaliana
  source 1..2133: mol_type = genomic DNA
atgtcctcctcttcttcatcatccacctctatgattgatttgatggccgctattatcaagggtgaaccagttatagtttctgatccagctaatgcttctgcctatgaatctgttgctgctgaattatcctccatgttgatcgaaaacagacaattcgctatgatcgtcactacctctattgctgttttgattggttgcatcgttatgttggtttggagaagatctggttctggtaactctaaaagagtcgaaccattgaagccattggttatcaaacctagagaagaagaaattgacgacggtagaaagaaggttaccattttctttggtactcaaaccggtactgctgaaggttttgctaaagctttgggtgaagaagctaaagccagatacgaaaagactagattcaagatcgttgacttggatgattacgctgcagatgatgatgaatacgaagaaaagttgaagaaagaagatgtcgcctttttcttcttggctacttatggtgatggtgaacctactgataatgctgctagattttacaagtggttcaccgaaggtaatgatagaggtgaatggttgaaaaacttgaagtacggtgttttcggtttgggtaatagacaatacgaacacttcaacaaggttgccaaggttgttgatgatatcttggttgaacaaggtgcccaaagattggttcaagttggtttaggtgatgatgaccaatgcatcgaagatgattttactgcttggagagaagctttgtggccagaattggatacaatcttgagagaagaaggtgatactgctgttgctactccatatactgctgctgttttagaatacagagtttccatccacgattccgaagatgctaagttcaacgatattaacatggctaacggtaacggttacaccgtttttgatgctcaacatccatacaaggctaacgttgctgttaagagagaattgcatactccagaatctgacagatcctgcattcatttggaattcgatattgctggttccggtttgacttacgaaactggtgatcatgttggtgttttgtgcgataacttgtctgaaactgttgatgaagccttgagattattggatatgtctccagatacctacttctccttgcatgccgaaaaagaagatggtactccaatctcttcatctttgccaccaccatttccaccatgtaatttgagaactgctttgaccagatacgcttgcttgttgtcatctccaaaaaagtctgctttggttgctttggctgctcatgcttcagatccaactgaagctgaaagattgaaacatttggcttctccagctggtaaggatgaatattctaaatgggttgttgaatcccaaagatccttgttggaagttatggctgaatttccatctgctaaaccaccattgggtgttttttttgctggtgttgctccaagattgcaacctagattctactctatttcctcctccccaaaaattgccgaaaccagaattcatgttacttgcgctttggtctacgaaaaaatgccaactggtagaatccataagggtgtttgttctacctggatgaagaatgctgttccttacgaaaagtccgaaaactgttcttctgctccaatcttcgttagacaatccaatttcaagttgccatccgattctaaggttccaattatcatgattggtccaggtactggtttggctccttttagaggttttttacaagaaagattggccttggtcgaatccggtgttgaattgggtccatctgttttgtttttcggttgcagaaacagaagaatggacttcatctacgaagaagaattacaaagattcgtcgaatcaggtgctttggcagaattgtcagttgctttttctagagaaggtccaacaaaagaatacgtccaacacaagatgatggataaggcttctgatatctggaacatgatttctcaaggtgcctacttgtatgtttgtggtgatgctaaaggtatggccagagatgttcatagatccttgcatacaattgcccaagaacaaggttctatggactctacaaaagcagaaggtttcgtcaagaacttgcaaacttctggtagatacttgagagatgtctgg

SEQ ID NO: 29 - DNA, 2121 bp, Lotus japonicus
  source 1..2121: mol_type = genomic DNA
atggaagaatcaagctccatgaagatttcgcctctggatctgatgtccgccatgatcaagggcacactcgacccttccaacgtctcctccacctccggcgccggctccgtcttcctcgagaatcgtgagttcgtcatggtgcttaccacctccatcgccgtcctcatcggatgcgtcgtcgttttcatttggcgcagatccaccggtaacaaggctaagtccatcgagcctcccaagcgcgtcgtcgagaagcttagcgacgaggctgaggttgacgacggtaccagaaaggtcaccatcttcttcggtactcagactggtactgctgaaggattcgccaaggcgattgcggaagaggcaaaagtgcgatacgaaaaagccaagttcaaaattgttgatatggatgattatgcccaggacgatgatgagtatgaggaaaagctcaagaaagagacactggcacttttcttcttagctacatatggtgatggtgagccaactgataatgcggcgagattttacaaatggtttctggagggagatgagaaagaagaaggatggcttcgaaatcttgagtatgctgtttttggtctggggaacaggcagtatgagcattttaataaggtcgccattgaagtggatgataagcttgctgattttggtgggaagcgtcttgtcaaagtaggtctaggagatgatgatcaatgcatagaagatgactttactgcatggaaagaagaattgtggccagcattggatgaattgcttagaggtgatgatgatacaactgtgtctacaccctatacggctgctgtgttggagtatcgtgttgttattcatgatccattagatgcatctgtcgatgaaaagaagtggcataatgttaatggccatgctattgtggatgctcaacatccagtcaggtcaaatgtggctgtgcgaaaggagcttcatactcctgtgtcagatcgttcttgcacacatttagaatttgacatttcaggcactggagttgcatatgaaacaggggaccatgttggtgtttactgtgagaatttatctgaaactgtggaagaagcagtaaggttactaggtttgtcaccagatacctatttctccgtccatactgatgatgaagatgggaaaccacttagtggaagctccttgccacctactttcccaccatgtactttaagaacagcaattgcccgatatgcagatgtcttgagttcacccaaaaagtctgttttgcttgccttagctgctcatgcatctaatccatctgaagccgaccgcctacgacatcttgcttcacctgctggaaaggatgaatattcagagtgggtgattgccagtcaaagaagtctccttgaggtcatggctgaatttccatcagccaaacctccaattggtgtctttttcgcagcaattgctcctcgcctgcagccaagattttattcgatctcatcatctcctaggatggctccatccagaattcacgttacctgtgcattagtgaatgataaaatgcccactggtaggattcataggggagtgtgttcaacatggatgaagaattctgtgccattggagaaaagtcaggactgcagttgggctccaatatttgttagacagtccaattttaaactccctgctgataataaagtgcctataatcatgataggtcctggcacaggattggctcctttcaggggtttcttgcaggaaagattagctctgaaagaggatggagctgaacttggcccctccgttttattcttcggatgcaggaatcgtcaaatggactacatctatgaagatgagttgaaccactttgtcaacagtggtgcgctttctgagctcattgttgccttctcacgggagggacctaccaaggaatatgtgcaacataaaatgatggagaaggcttcggatatttggaacatgatatctcagggagcttacatttatgtgtgtggggatgccaagggcatggctagggatgtgcaccgtactctgcacacaattttgcaagagcagggttctctcgatagttccaaggctgagggtatggttaagaacctacaattgaatggtaggtatttgcgtgatgtatggtga

SEQ ID NO: 30 - DNA, 2145 bp, Catharanthus roseus
  source 1..2145: mol_type = genomic DNA
atggattctagctcggagaagttgtcgccgttcgaattgatgagcgcgatcttgaagggagctaaattagatgggtctaactcttcagattctggcgtagctgtgtcgccggcagttatggctatgttgttggagaataaggagttagtgatgattttgactacttcagtggcggttttgatcggttgtgtcgtagttttgatatggcggcgatcttccggatcgggtaaaaaagtcgtggagcctccgaagctcatagtgcctaaatctgttgtagaaccggaggaaattgatgaagggaagaagaaatttaccatattttttggaacacaaactggaacagctgaaggcttcgctaaggctctagctgaggaagccaaagctcgatatgaaaaggcagttatcaaagtgattgatatagatgattatgcggctgatgatgaagaatacgaggagaaattcagaaaagagaccttggcatttttcatcttggccacgtatggagatggtgagccaaccgacaatgctgcaaggttctacaaatggtttgtagagggaaatgatagaggggactggctaaagaatctgcaatatggagtttttggccttggtaacagacaatatgagcatttcaacaagattgctaaagtggtggatgagaaagttgctgaacagggtggtaagcggattgttccattggttctgggagacgatgaccagtgcattgaagatgactttgctgcatggcgtgagaatgtatggcctgagttggataacttgctccgggatgaggatgatacaactgtttctacaacctacactgctgctattccagaatatcgtgttgtgttccctgacaaatcagattcacttatttcagaagcaaatggccatgccaatggttatgctaatggcaacaccgtatatgatgcccagcatccttgcagatctaatgttgcagtgaggaaggagcttcatactccagcatctgatcgttcttgcacccatttggattttgacattgctggcactggcctttcatatggaactggagatcatgttggagtgtactgtgataatctatctgaaaccgtggaggaggctgagagattactgaatttacccccagaaacttatttctcgcttcatgctgataaagaggatggaaccccacttgctgggagctcattgcctcctcctttcccaccttgtactctaagaaccgccctcactcgttatgcagatctcttaaatactcctaagaagtctgctttgttagctctagcagcttatgcatctgatccaaatgaggccgatcgtctaaaatatcttgcttctccagccggaaaggatgaatatgctcagtcactagttgcaaatcagagaagcctcctcgaggtcatggctgaatttccatcagcaaagcctcctcttggagtattctttgcagcaattgctccacgcctccaacccagattctattctatatcgtcttctccaaggatggcaccatctagaattcatgtcacttgtgcacttgtttatgaaaaaacacctggaggacgaattcacaagggtgtgtgttcgacatggatgaagaatgccattccattggaggaaagccgtgactgcagctgggctcctatctttgtcaggcagtctaacttcaaactccctgccgatcctaaagtgcctgttataatgatcggccctggtactggactagctcccttcagaggattccttcaggaaagattagctctgaaggaagaaggagctgaacttggtactgcagttttcttttttggatgcaggaaccgcaaaatggattacatctatgaagatgagctaaaccatttccttgaaattggtgcactttccgagctacttgttgctttctcacgtgagggacccactaagcagtatgtgcaacacaagatggcagaaaaggcttctgatatttggaggatgatttctgatggagcatatgtttacgtctgcggtgatgccaaaggcatggccagggatgtccacagaactctccacaccattgctcaagagcagggatcgatggatagcacacaggctgagggttttgtgaagaatctgcaaatgaccggaaggtatctccgagatgtctggtga

SEQ ID NO: 31 - DNA, 1815 bp, Saccharomyces cerevisiae
  source 1..1815: mol_type = genomic DNA
atgacggagactaaggatttgttgcaagacgaagagtttcttaagatccgcagactcaattccgcagagcccaacaaacggcattcggtcacgtacgataacgtgatcctgccacaggagtccatggaggtttctccacggtcgtctaccacgtcgctggtggagccagtggagtcgagtgaggcggaacgtgtggcagggaagcaggagcaggaggaggagtaccctgtggatgcccacatgcaaaagtacctttcacacctgaagagcaagtctcggtcgaggttccaccgaaaggatgctagcaagtatgtgtcgttttttggggacgtgagttttgatcctcgccccacgctcctggacagcgccatcaatgtgcccttccagacgactttcaaaggtccggtgctggagaaacagctcaaaaatttacagttgacaaagaccaaggccaaggccacggtgaaggctacggtgaagactacggagaaaacggacaaggcagatgcccccccaggagaaaaactggagtcgaacttttcagggatctacgtgttcgcatggatgttcttgggctggattgccatcaggtgctgcacagattactatgcgtcgtacggcagtgcatggaataagctggaaatcgtgcagtacatgacaacggacttgttcacgatcgcaatgttggacctggcaatgttcctgtgcactttcttcgtggttttcgtgcactggctggtgaaaaagcggatcatcaactggaagtggactgggttcgttgcagtgagcatcttcgagttggctttcatccccgtgacgttccccatttacgtctactactttgatttcaactgggtcacgagaatcttcctgttcctgcactccgtggtgtttgttatgaagagccactcgtttgccttttacaacgggtatctttgggacataaagcaggaactcgagtactcttccaaacagttgcaaaaatacaaggaatccttgtccccagagacccgcgagattctgcaaaaaagttgcgacttttgccttttcgaattgaactaccagaccaaggataacgacttccccaacaacatcagttgcagcaatttcttcatgttctgtctgttccccgtcctcgtgtaccagatcaactacccaagaacgtcgcgcatcagatggaggtatgtgttggagaaggtatgcgccatcattggcaccatcttcctcatgatggtcaccgcacagttcttcatgcacccggtggccatgcgctgtatccagttccacaacacgcccaccttcggcggctggatccccgccacgcaagagtggttccacctgctcttcgacatgattccgggcttcactgttctgtacatgctcacgttttacatgatatgggacgctttattgaattgcgtggcggagttgaccaggtttgcggacagatatttctacggcgactggtggaattgcgtttcgtttgaagagtttagcagaatctggaacgtccccgttcacaaatttttactaagacacgtgtaccacagctccatgggcgcattgcatttgagcaagagccaagctacattattcacttttttcttgagtgccgtgttccacgaaatggccatgttcgccattttcagaagggttagaggatatctgttcatgttccaactgtcgcagtttgtgtggactgctttgagcaacaccaagtttctacgggcaagaccgcagttgtccaacgttgtcttttcgtttggtgtctgttcagggcccagtatcattatgacgttgtacctgaccttatga

SEQ ID NO: 32 - DNA, 1929 bp, Saccharomyces cerevisiae
  source 1..1929: mol_type = genomic DNA
atggacaagaagaaggatctactggagaacgaacaatttctccgcatccaaaagctcaacgctgccgatgcgggcaaaagacaatctataacagtggacgacgagggcgaactatatgggttagacacctccggcaactcaccagccaatgaacacacagctaccacaattacacagaatcacagcgtggtggcctcaaacggagacgtcgcattcatcccaggaactgctaccgaaggcaatacagagattgtaactgaagaagtgattgagaccgatgataacatgttcaagacccatgtgaagactttaagctccaaagagaaggcacggtataggcaagggtcctctaactttatatcgtatttcgatgatatgtcatttgaacacaggcccagtatattagatgggtcagttaacgagcccttcaagaccaaattcgtgggacctactttagaaaaggagatcagaagaagggagaaagagctaatggccatgcgcaaaaatttacaccaccgcaagtcctccccagatgctgtcgactcagtagggaaaaatgatggcgccgccccaactactgttccaactgccgccacctcagaaacggtggtcaccgttgaaaccaccataatttcatccaatttctccgggttgtacgtggcgttttggatggctattgcatttggtgctgtcaaggctttaatagactattattaccagcataatggtagcttcaaggattcggagatcttgaaatttatgactacgaatttgttcactgtggcatccgtagatcttttgatgtatttgagcacttattttgtcgttggaatacaatacttatgcaagtggggggtcttgaaatggggcactaccggctggatcttcacctcaatttacgagtttttgtttgttatcttctacatgtatttaacagaaaacatcctaaaactacactggctgtccaagatcttcctttttttgcattctttagttttattgatgaaaatgcattctttcgccttctacaatggctatctatggggtataaaggaagaactacaattttccaaaagcgctcttgccaaatacaaggattctataaatgatccaaaagttattggtgctcttgagaaaagctgtgagttttgtagttttgaattgagctctcagtctttaagcgaccaaactcaaaaattccccaacaatatcagtgcaaaaagctttttttggttcaccatgtttccaaccctaatttaccaaattgaatatccaagaactaaggaaatcagatggagctacgtattagaaaagatctgcgccatcttcggtaccattttcttaatgatgatagatgctcaaatcttgatgtatcctgtagcaatgagagcattggctgtgcgcaattctgaatggactggtatattggatagattattgaaatgggttggattgctcgttgatatcgtcccagggtttatcgtgatgtacatcttggacttctatttgatttgggatgccattttgaactgtgtggctgaattgacaagatttggcgacagatatttctacggtgactggtggaattgtgttagttgggcagacttcagtagaatttggaacatcccagtgcataagtttttgttaagacatgtttaccatagttcaatgagttcattcaaattgaacaagagtcaagcaactttgatgacctttttcttaagttccgtcgttcatgaattagcaatgtacgttatcttcaagaaattgaggttttacttgttcttcttccaaatgctgcaaatgccattagtagctttaacaaatactaaattcatgaggaacagaaccataatcggaaatgttattttctggctcggtatctgcatgggaccaagtgtcatgtgtacgttgtacttgacattctaa

SEQ ID NO: 33 - DNA, 2286 bp, Artemisia annua
  source 1..2286: mol_type = genomic DNA
atgtggagattgaaaatagcagaagggcgcaatgatccgtacctgtacagcacaaacaactttgttggaaggcaaatatgggagtttgacccgaattatggaactccagaagagcgtgctgaggtggaacaagctcgtgttgatttctggaatcacagacacgaagttaaacccagtagtgatgttctttggcgcatgcagtttttaagagagaagggatttgaacagacgataccccaagtgaaaatagaggacggtgaagaaatatcgtatgaaaaagctacaaccacattaaggagatcggttaacttctttgcagctttgcaggccgatgatggtcactggccagctgaaaatgctggtcccttgtatttcatgcagcccttggtcatatgcttgtatattactgggcaccttaacactgttttcccagcagaatatcgaaaagaaattctacggtacatatactgtcatcagaatgaagacggtgggtggggatttcacattgagggtcatagtacaatgttctgcaccaccctcagttacatttgtatgcgtcttttgggggaaggtcgtgatggtggtctggatggtgcatgcaccaaagctcgtaaatggattctagaccatggcagtgtcacgaccattccatcttggggtaaaacatggctttcgatactcggtgtatgtgagtgggcaggaaccaacccaatgccccccgagttctggatcctcccttctttcttacctatgtatccagcaaaaatgtggtgttactgtcgacttgtatatatgccaatgtcatacctatacggaaaaagattcgtgggtcctattaccccattaattctacaacttagagatgaactctatgcacaaccgtatgatgaaattaaatggaggagcataaggcatttgtgtgcaaaggaagacttatactatcctcatcctttactacaagatttgatgtgggatagtctttatgtttttactgagcctgtactaaatcactggccatttaataaattgcgcgagaaagcgctacaaacaacaatgaagcatatccattatgaagatgaaaacagcagatatatcacaattggttctgtagagaaggcactgtgtatgctcgcatgctgggttgaggatccaaatggagtttgttttaaaaagcatattgctcgtattccagattatctctgggttgctgaagatggaatgaaaatgcagagcttcggcagtcaggagtgggatgctggttttgctattcaagctctaatggcaactgatcttactgatgaaattgggtctactttgatgaaaggacatgaatttattaaagcttcacaggttaaagacaatccttcgggtgactttaaaagcatgcatcgacatatttctaaagggtcatggacattttcagatcaagatcatggatggcaagtttcagactgtactgccgaagcattaaagtgttgcctcctatttgcaacgatgccaccagaaatagttggggagaaaatgaaacctgaacaactgaacgatgctgtcaacgtaatactttccttacagagcaaaaatggcgggctagccgcatgggaaccagcaggatcgtcagaatggctggagattctcaatcctacagagttctttgctgatatcgtcatagagcatgagtacgttgagtgcacatcatcagcaattcaggctctggttatgttcaaaaagaaatacccggggcataggaagaaagagatagaaaattttcttttgggttcaagtggatacctggagaaaatacagatggaagatggttcatggtatggaaactggggtgtgtgttttacatacggtacatggtttgctcttggaggactatcagcggttgggaaaacatatgacaattgtccagcaatccgtaaagctgtcaaatttctcctggaaacccagctggaagatggtggttggggagaaagctacaaatcctgtccagaaaagaaatatatacccctggaaggaggtcggtccaacttggtacacactgcatgggctatgatgggactaattcattctcgacaggccgagagagatgcaacacccctacacagagcagccaaactattaatcaactcccagttggaaactggtgattttccacaacaggaaatagctggagtctttatgaagaattgcatgctacactatgccttatacagaaatatatacccgatgtgggctctagctgattatcgcaagcaggtgctgccacagctcaaaggcacctag

SEQ ID NO: 34 - DNA, 2310 bp, Panax ginseng
  source 1..2310: mol_type = genomic DNA
atgtggaagctgaaggttgctcaaggaaatgacccttacttgtattctacgaacaactttgtcggaaggcaatattgggagtttcaacctgatgcgggtacgcctgaagaaagggaggaagtagaaaaagctagaaaggattatgtaaataataagaagttacacggcatacacccatgctctgacatgctgatgcgtagacagctaattaaggaaagtggcatagatctattaagcattcctcctctacgtttggatgaaaatgaacaggtaaattatgacgcagtgacaactgcagtaaagaaagccttaaggcttaatagagccattcaagctcacgacggccactggccagcggaaaacgctggcagtcttttatatacgcctccgttgataattgcactatatatctctggaacaattgacacaattctaacaaagcagcataagaaagaattgatccgtttcgtatacaaccatcagaatgaagacggtgggtggggatcatatatcgaaggccactctacgatgataggttccgtactaagttacgtcatgttaagactattgggcgaaggcttagccgaaagtgatgacggcaatggagctgtagagagaggcaggaagtggatcttggaccatggaggtgcagcaggcatacctagctggggaaagacctacctagctgttttaggagtatatgaatgggaaggttgcaatccattgccccctgaattttggctattccctagttcattcccttttcatcccgcgaagatgtggatttattgccgttgtacctatatgccaatgtcatatctatatggaaaaaggtatcacggtccgatcaccgatctggtattatcattaaggcaagaaatctataatatcccgtacgaacagattaagtggaaccaacagaggcataactgctgtaaggaagatctttattacccacacaccttagttcaagacctagtgtgggacggtcttcattacttcagtgagccatttttgaaacgttggccatttaataaactaagaaagcgtgggttgaagcgtgtcgtagaactaatgagatatggtgcaactgaaacaaggttcataacaaccggtaatggggaaaaggctctacaaattatgagctggtgggctgaagacccgaacggtgacgagtttaagcaccaccttgctaggattccggacttcttatggatcgccgaggacggcatgaccgtccagtcattcggttcacagctatgggattgtatactggctacgcaggctataatcgctactaacatggttgaagagtatggcgacagcttaaaaaaggcgcatttcttcataaaggaatcacagataaaagagaatcctagaggggatttcttaaagatgtgtaggcagtttacaaaaggtgcttggacgttttctgaccaagaccatgggtgcgtggtaagcgattgcaccgccgaggccttgaagtgcttgctgctactatcacagatgccccaggatattgttggggagaaacctgaggtcgaaaggttatacgaagctgtcaacgttcttctatacctacaatccagagtgtcaggcggtttcgcagtctgggagcctcctgtgcccaagccttatctggaaatgttaaatccatccgaaatttttgccgatattgttgtggaaagggagcacattgagtgcaccgcctctgttattaaaggcctaatggcttttaaatgcttacatccagggcaccgtcaaaaagaaatcgaagatagtgtagcaaaagcgattaggtatctagagagaaatcaaatgcctgacggcagctggtacgggttttgggggatatgctttttatatggaacattctttactttatccggatttgccagtgcggggagaacctatgataactcagaagccgtaaggaaaggtgttaaattcttcctgtctactcaaaacgaggaaggtgggtggggggagtcattagaaagttgtccatctgagaagtttacgccgctgaaggggaacaggactaacttggtacagacatcctgggccatgttaggactgatgttcggaggacaagcagagagggatccaactccgctgcacagagcagctaaattgttaataaacgcacagatggacaacggggactttccacagcaggagattactggagtatactgtaaaaatagcatgctgcactacgcggaatataggaacatttttcctttgtgggccctgggcgaatacaggaagcgtgtctggttgcctaaacaccagcagcttaaaatataa

SEQ ID NO: 35 - DNA, 2331 bp, synthetic construct
  misc_feature 1..2331: note = derived from yeast Simo-t-ERG7
  source 1..2331: mol_type = other DNA
atgacagaattttattctgacacaatcggtctaccaaagacagatccacgtctttggagactgagaactgatgagctaggccgagaaagctgggaatatttaacccctcagcaagccgcaaacgacccaccatccactttcacgcagtggcttcttcaagatcccaaatttcctcaacctcatccagaaagaaataagcattcaccagatttttcagccttcgatgcgtgtcataatggtgcatcttttttcaaactgcttcaagagcctgactcaggtatttttccgtgtcaatataaaggacccatgttcatgacaatcggttacgtagccgtaaactatatcgccggtattgaaattcctgagcatgagagaatagaattaattagatacatcgtcaatacagcacatccggttgatggtggctggggtctacattctgttgacaaatccaccgtgtttggtacagtattgaactatgtaatcttacgtttattgggtctacccaaggaccacccggtttgcgccaaggcaagaagcacattgttaaggttaggcggtgctattggatcccctcactggggaaaaatttggctaagtgcactaaacttgtataaatgggaaggtgtgaaccctgcccctcctgaaacttggttacttccatattcactgcccatgcatccggggagatggtgggttcatactagaggtgtttacattccggtcagttacctgtcattggtcaaattttcttgcccaatgactcctcttcttgaagaactgaggaatgaaatttacactaaaccgtttgacaagattaacttctccaagaacaggaataccgtatgtggagtagacctatattacccccattctactactttgaatattgcgaacagccttgtagtattttacgaaaaatacctaagaaaccggttcatttactctctatccaagaagaaggtttatgatctaatcaaaacggagttacagaatactgattccttgtgtatagcacctgttaaccaggcgttttgcgcacttgtcactcttattgaagaaggggtagactcggaagcgttccagcgtctccaatataggttcaaggatgcattgttccatggtccacagggtatgaccattatgggaacaaatggtgtgcaaacctgggattgtgcgtttgccattcaatactttttcgtcgcaggcctcgcagaaagacctgaattctataacacaattgtctctgcctataaattcttgtgtcatgctcaatttgacaccgagtgcgttccaggtagttatagggataagagaaagggggcttggggcttctcaacaaaaacacagggctatacagtggcagattgcactgcagaagcaattaaagccatcatcatggtgaaaaactctcccgtctttagtgaagtacaccatatgattagcagtgaacgtttatttgaaggcattgatgtgttattgaacctacaaaacatcggatcttttgaatatggttcctttgcaacctatgaaaaaatcaaggccccactagcaatggaaaccttgaatcctgctgaagtttttggtaacataatggtagaatacccatacgtggaatgtactgattcatccgttctggggttgacatattttcacaagtacttcgactataggaaagaggaaatacgtacacgcatcagaatcgccatcgaattcataaaaaaatctcaattaccagatggaagttggtatggaagctggggtatttgttttacatatgccggtatgtttgcattggaggcattacacaccgtgggggagacctatgagaattcctcaacggtaagaaaaggttgcgacttcttggtcagtaaacagatgaaggatggcggttggggggaatcaatgaagtccagtgaattacatagttatgtggatagtgaaaaatcgctagtcgttcaaaccgcatgggcgctaattgcacttcttttcgctgaatatcctaataaagaagtcatcgaccgcggtattgaccttttaaaaaatagacaagaagaatccggggaatggaaatttgaaagtgtagaaggtgttttcaaccactcttgtgcaattgaatacccaagttatcgattcttattccctattaaggcattaggtatgtacagcagggcatatgaaacactacgcttggagcaggtgctggtgctggtgctggagcaattctgtctaaaggtgaagaattattcactggtgttgtcccaattttggttgaattagatggtgatgttaatggtcacaaattttctgtctccggtgaaggtgaaggtg

SEQ ID NO: 36 - DNA, 2678 bp, synthetic construct
  misc_feature 1..2678: note = derived from yeast, Simo-m-ERG7
  source 1..2678: mol_type = other DNA
atgacagaattttattctgacacaatcggtctaccaaagacagatccacgtctttggagactgagaactgatgagctaggccgagaaagctgggaatatttaacccctcagcaagccgcaaacgacccaccatccactttcacgcagtggcttcttcaagatcccaaatttcctcaacctcatccagaaagaaataagcattcaccagatttttcagccttcgatgcgtgtcataatggtgcatcttttttcaaactgcttcaagagcctgactcaggtatttttccgtgtcaatataaaggacccatgttcatgacaatcggttacgtagccgtaaactatatcgccggtattgaaattcctgagcatgagagaatagaattaattagatacatcgtcaatacagcacatccggttgatggtggctggggtctacattctgttgacaaatccaccgtgtttggtacagtattgaactatgtaatcttacgtttattgggtctacccaaggaccacccggtttgcgccaaggcaagaagcacattgttaaggttaggcggtgctattggatcccctcactggggaaaaatttggctaagtgcactaaacttgtataaatgggaaggtgtgaaccctgcccctcctgaaacttggttacttccatattcactgcccatgcatccggggagatggtgggttcatactagaggtgtttacattccggtcagttacctgtcattggtcaaattttcttgcccaatgactcctcttcttgaagaactgaggaatgaaatttacactaaaccgtttgacaagattaacttctccaagaacaggaataccgtatgtggagtagacctatattacccccattctactactttgaatattgcgaacagccttgtagtattttacgaaaaatacctaagaaaccggttcatttactctctatccaagaagaaggtttatgatctaatcaaaacggagttacagaatactgattccttgtgtatagcacctgttaaccaggcgttttgcgcacttgtcactcttattgaagaaggggtagactcggaagcgttccagcgtctccaatataggttcaaggatgcattgttccatggtccacagggtatgaccattatgggaacaaatggtgtgcaaacctgggattgtgcgtttgccattcaatactttttcgtcgcaggcctcgcagaaagacctgaattctataacacaattgtctctgcctataaattcttgtgtcatgctcaatttgacaccgagtgcgttccaggtagttatagggataagagaaagggggcttggggcttctcaacaaaaacacagggctatacagtggcagattgcactgcagaagcaattaaagccatcatcatggtgaaaaactctcccgtctttagtgaagtacaccatatgattagcagtgaacgtttatttgaaggcattgatgtgttattgaacctacaaaacatcggatcttttgaatatggttcctttgcaacctatgaaaaaatcaaggccccactagcaatggaaaccttgaatcctgctgaagtttttggtaacataatggtagaatacccatacgtggaatgtactgattcatccgttctggggttgacatattttcacaagtacttcgactataggaaagaggaaatacgtacacgcatcagaatcgccatcgaattcataaaaaaatctcaattaccagatggaagttggtatggaagctggggtatttgttttacatatgccggtatgtttgcattggaggcattacacaccgtgggggagacctatgagaattcctcaacggtaagaaaaggttgcgacttcttggtcagtaaacagatgaaggatggcggttggggggaatcaatgaagtccagtgaattacatagttatgtggatagtgaaaaatcgctagtcgttcaaaccgcatgggcgctaattgcacttcttttcgctgaatatcctaataaagaagtcatcgaccgcggtattgaccttttaaaaaatagacaagaagaatccggggaatggaaatttgaaagtgtagaaggtgttttcaaccactcttgtgcaattgaatacccaagttatcgattcttattccctattaaggcattaggtatgtacagcagggcatatgaaacactacgcttggagcaggtgctggtgctggtgctggagcaattctgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttac

SEQ ID NO: 37 - protein, 759 aa, synthetic construct; identical to SEQ ID NO: 19
  REGION 1..759: note = yeast modified

SEQ ID NO: 38 - DNA, 2985 bp, synthetic construct
  misc_feature 1..2985: note = derived from yeast Simo-gt-ERG7
  source 1..2985: mol_type = other DNA
atgacagaattttattctgacacaatcggtctaccaaagacagatccacgtctttggagactgagaactgatgagctaggccgagaaagctgggaatatttaacccctcagcaagccgcaaacgacccaccatccactttcacgcagtggcttcttcaagatcccaaatttcctcaacctcatccagaaagaaataagcattcaccagatttttcagccttcgatgcgtgtcataatggtgcatcttttttcaaactgcttcaagagcctgactcaggtatttttccgtgtcaatataaaggacccatgttcatgacaatcggttacgtagccgtaaactatatcgccggtattgaaattcctgagcatgagagaatagaattaattagatacatcgtcaatacagcacatccggttgatggtggctggggtctacattctgttgacaaatccaccgtgtttggtacagtattgaactatgtaatcttacgtttattgggtctacccaaggaccacccggtttgcgccaaggcaagaagcacattgttaaggttaggcggtgctattggatcccctcactggggaaaaatttggctaagtgcactaaacttgtataaatgggaaggtgtgaaccctgcccctcctgaaacttggttacttccatattcactgcccatgcatccggggagatggtgggttcatactagaggtgtttacattccggtcagttacctgtcattggtcaaattttcttgcccaatgactcctcttcttgaagaactgaggaatgaaatttacactaaaccgtttgacaagattaacttctccaagaacaggaataccgtatgtggagtagacctatattacccccattctactactttgaatattgcgaacagccttgtagtattttacgaaaaatacctaagaaaccggttcatttactctctatccaagaagaaggtttatgatctaatcaaaacggagttacagaatactgattccttgtgtatagcacctgttaaccaggcgttttgcgcacttgtcactcttattgaagaaggggtagactcggaagcgttccagcgtctccaatataggttcaaggatgcattgttccatggtccacagggtatgaccattatgggaacaaatggtgtgcaaacctgggattgtgcgtttgccattcaatactttttcgtcgcaggcctcgcagaaagacctgaattctataacacaattgtctctgcctataaattcttgtgtcatgctcaatttgacaccgagtgcgttccaggtagttatagggataagagaaagggggcttggggcttctcaacaaaaacacagggctatacagtggcagattgcactgcagaagcaattaaagccatcatcatggtgaaaaactctcccgtctttagtgaagtacaccatatgattagcagtgaacgtttatttgaaggcattgatgtgttattgaacctacaaaacatcggatcttttgaatatggttcctttgcaacctatgaaaaaatcaaggccccactagcaatggaaaccttgaatcctgctgaagtttttggtaacataatggtagaatacccatacgtggaatgtactgattcatccgttctggggttgacatattttcacaagtacttcgactataggaaagaggaaatacgtacacgcatcagaatcgccatcgaattcataaaaaaatctcaattaccagatggaagttggtatggaagctggggtatttgttttacatatgccggtatgtttgcattggaggcattacacaccgtgggggagacctatgagaattcctcaacggtaagaaaaggttgcgacttcttggtcagtaaacagatgaaggatggcggttggggggaatcaatgaagtccagtgaattacatagttatgtggatagtgaaaaatcgctagtcgttcaaaccgcatgggcgctaattgcacttcttttcgctgaatatcctaataaagaagtcatcgaccgcggtattgaccttttaaaaaatagacaagaagaatccggggaatggaaatttgaaagtgtagaaggtgttttcaaccactcttgtgcaattgaatacccaagttatcgattcttattccctattaaggcattaggtatgtacagcagggcatatgaaacactacgcttggagcaggtgctggtgctggtgctggagcaattctgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaattgccaatgtcttgtgcacaagaatctattacttctttgtacaaga

SEQ ID NO: 39 - protein, 759 aa, synthetic construct; identical to SEQ ID NO: 19
  REGION 1..759: note = yeast modified

SEQ ID NO: 40 - DNA, 2287 bp, synthetic construct
  misc_feature 1..2287: note = derived from yeast, Simo-o-ERG7
  source 1..2287: mol_type = other DNA
atgacagaattttattctgacacaatcggtctaccaaagacagatccacgtctttggagactgagaactgatgagctaggccgagaaagctgggaatatttaacccctcagcaagccgcaaacgacccaccatccactttcacgcagtggcttcttcaagatcccaaatttcctcaacctcatccagaaagaaataagcattcaccagatttttcagccttcgatgcgtgtcataatggtgcatcttttttcaaactgcttcaagagcctgactcaggtatttttccgtgtcaatataaaggacccatgttcatgacaatcggttacgtagccgtaaactatatcgccggtattgaaattcctgagcatgagagaatagaattaattagatacatcgtcaatacagcacatccggttgatggtggctggggtctacattctgttgacaaatccaccgtgtttggtacagtattgaactatgtaatcttacgtttattgggtctacccaaggaccacccggtttgcgccaaggcaagaagcacattgttaaggttaggcggtgctattggatcccctcactggggaaaaatttggctaagtgcactaaacttgtataaatgggaaggtgtgaaccctgcccctcctgaaacttggttacttccatattcactgcccatgcatccggggagatggtgggttcatactagaggtgtttacattccggtcagttacctgtcattggtcaaattttcttgcccaatgactcctcttcttgaagaactgaggaatgaaatttacactaaaccgtttgacaagattaacttctccaagaacaggaataccgtatgtggagtagacctatattacccccattctactactttgaatattgcgaacagccttgtagtattttacgaaaaatacctaagaaaccggttcatttactctctatccaagaagaaggtttatgatctaatcaaaacggagttacagaatactgattccttgtgtatagcacctgttaaccaggcgttttgcgcacttgtcactcttattgaagaaggggtagactcggaagcgttccagcgtctccaatataggttcaaggatgcattgttccatggtccacagggtatgaccattatgggaacaaatggtgtgcaaacctgggattgtgcgtttgccattcaatactttttcgtcgcaggcctcgcagaaagacctgaattctataacacaattgtctctgcctataaattcttgtgtcatgctcaatttgacaccgagtgcgttccaggtagttatagggataagagaaagggggcttggggcttctcaacaaaaacacagggctatacagtggcagattgcactgcagaagcaattaaagccatcatcatggtgaaaaactctcccgtctttagtgaagtacaccatatgattagcagtgaacgtttatttgaaggcattgatgtgttattgaacctacaaaacatcggatcttttgaatatggttcctttgcaacctatgaaaaaatcaaggccccactagcaatggaaaccttgaatcctgctgaagtttttggtaacataatggtagaatacccatacgtggaatgtactgattcatccgttctggggttgacatattttcacaagtacttcgactataggaaagaggaaatacgtacacgcatcagaatcgccatcgaattcataaaaaaatctcaattaccagatggaagttggtatggaagctggggtatttgttttacatatgccggtatgtttgcattggaggcattacacaccgtgggggagacctatgagaattcctcaacggtaagaaaaggttgcgacttcttggtcagtaaacagatgaaggatggcggttggggggaatcaatgaagtccagtgaattacatagttatgtggatagtgaaaaatcgctagtcgttcaaaccgcatgggcgctaattgcacttcttttcgctgaatatcctaataaagaagtcatcgaccgcggtattgaccttttaaaaaatagacaagaagaatccggggaatggaaatttgaaagtgtagaaggtgttttcaaccactcttgtgcaattgaatacccaagttatcgattcttattccctattaaggcattaggtatgtacagcagggcatatgaaacactacgcttggagcaggtgctggtgctggtgctggagcaattctgtctaaaggtgaagaattattcactggtgttgtcccaattttggttgaattagggatccc

SEQ ID NO: 41 - DNA, 6135 bp, synthetic construct
  misc_feature 1..6135: note = Expression Cassette
  source 1..6135: mol_type = other DNA
ttccggatattgtgttaaaatttcaagtatgctactaatggggtaaagttgcatgattagcagagacatatggcttgttatggttcggcttcctcatttttcatgcttagtttttgtccatctcattgtacatttctgaatcctaatgcatgactccctaacattactattaaattctcaatagtgaagaataagcaaaatgggaaccatgataatttctagctttctctccacccctattttaatttgcaatcatatatagtactttcaatagcatcttttctagatttgatatctgcggaaacttttcgtttataatcgtttaggtgaaaagtttttatatcggttatttacagatatacattttctcaaaaaaaaaaaaatataatacatgccctcagcttttaataaagccattgagcacaagcctctccagtaatgtactgctgtgcccaataaccttaccaataatcgtcgcccacaaagaaagtacaaaacagatgacagaattttattctgacacaatcggtctaccaaagacagatccacgtctttggagactgagaactgatgagctaggccgagaaagctgggaatatttaacccctcagcaagccgcaaacgacccaccatccactttcacgcagtggcttcttcaagatcccaaatttcctcaacctcatccagaaagaaataagcattcaccagatttttcagccttcgatgcgtgtcataatggtgcatcttttttcaaactgcttcaagagcctgactcaggtatttttccgtgtcaatataaaggacccatgttcatgacaatcggttacgtagccgtaaactatatcgccggtattgaaattcctgagcatgagagaatagaattaattagatacatcgtcaatacagcacatccggttgatggtggctggggtctacattctgttgacaaatccaccgtgtttggtacagtattgaactatgtaatcttacgtttattgggtctacccaaggaccacccggtttgcgccaaggcaagaagcacattgttaaggttaggcggtgctattggatcccctcactggggaaaaatttggctaagtgcactaaacttgtataaatgggaaggtgtgaaccctgcccctcctgaaacttggttacttccatattcactgcccatgcatccggggagatggtgggttcatactagaggtgtttacattccggtcagttacctgtcattggtcaaattttcttgcccaatgactcctcttcttgaagaactgaggaatgaaatttacactaaaccgtttgacaagattaacttctccaagaacaggaataccgtatgtggagtagacctatattacccccattctactactttgaatattgcgaacagccttgtagtattttacgaaaaatacctaagaaaccggttcatttactctctatccaagaagaaggtttatgatctaatcaaaacggagttacagaatactgattccttgtgtatagcacctgttaaccaggcgttttgcgcacttgtcactcttattgaagaaggggtagactcggaagcgttccagcgtctccaatataggttcaaggatgcattgttccatggtccacagggtatgaccattatgggaacaaatggtgtgcaaacctgggattgtgcgtttgccattcaatactttttcgtcgcaggcctcgcagaaagacctgaattctataacacaattgtctctgcctataaattcttgtgtcatgctcaatttgacaccgagtgcgttccaggtagttatagggataagagaaagggggcttggggcttctcaacaaaaacacagggctatacagtggcagattgcactgcagaagcaattaaagccatcatcatggtgaaaaactctcccgtctttagtgaagtacaccatatgattagcagtgaacgtttatttgaaggcattgatgtgttattgaacctacaaaacatcggatcttttgaatatggttcctttgcaacctatgaaaaaatcaaggccccactagcaatggaaaccttgaatcctgctgaagtttttggtaacataatggtagaatacccatacgtggaatgtactgattcatccgttctggggttgacatattttcacaagtacttcgactataggaaagaggaaatacgtacacgcatcagaatcgccatcgaattcataaaaaaatctcaattaccagatggaagttggtatggaagctggggtatttgttttacatatgccggtatgtttgcattggaggcattacacaccgtgggggagacctatgagaattcctcaacggtaagaaaaggttgcgacttcttggtcagtaaacagatgaaggatggcggttggggggaatcaatgaagtccagtgaattacatagttatgtggatagtgaaaaatcgctagtcgttcaaaccgcatgggcgctaattgcacttcttttcgctgaatatcctaataaagaagtcatcgaccgcggtattgaccttttaaaaaatagacaagaagaatccggggaatggaaatttgaaagtgtagaaggtgttttcaaccactcttgtgcaattgaatacccaagttatcgattcttattccctattaaggcattaggtatgtacagcagggcatatgaaacactacgcttggagcaggtgctggtgctggtgctggagcaattctgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaattgccaatgtcttgtgcacaagaatctattacttctttgtacaagaaggctggttctgaaaacttgtacttccaattccacaagtctggtgcttggaagttgccagtttctttggttaagagagggatcgataagcttgattataaagaacagcttcaggcttggcggtgggaaagagaaattgatgagagaaatcgcccactttctgatgaggaattagatgctatgttcccagaaggatataaggtacttcctcctccagctggttatgttcctattcgaactccagctcatatggatcgaattcctgcagtagcaggtgctggtgctggtgctggagcaatgtctgaattaattaaagaaaatatgcatatgaaattatatatggaaggtacagttaataatcatcattttaaatgtacatctgaaggtgaaggtaaaccatatgaaggtacacaaacaatgagaattaaagttgttgaaggtggtccattaccatttgcttttgatattttagctacatcttttatgtatggttctaagacatttattaatcatacacaaggtattccagatttttttaaacaatcttttccagaaggttttacatgggaaagagttacaacatatgaagatggtggtgttttaacagctacacaagatacatctttacaagatggttgtttaatttataatgttaaaattagaggtgttaattttccatctaatggtccagttatgcaaaaaaaaacattaggttgggaagcttctacagaaatgttatatccagctgatggtggtttagaaggtaggtctgatatggctttaaaattagttggtggtggtcatttaatttgtaatttgaaaacaacatataggtctaaaaaaccagctaaaaatttaaaaatgccaggtgtttattatgttgatagaagattagaaagaattaaagaagctgataaagaaacatatgttgaacaacatgaagttgctgttgctagatattgtgatttaccatctaaattaggtcataaaggatcctaagcgcgccagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataatctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggv

SEQ ID NO: 42 - protein, 1300 aa, synthetic construct
  REGION 1..1300: note = ERG7 in expression cassette, derived from yeast
MTEFYSDTIGLPKTDPRLWRLRTDELGRESWEYLTPQQAANDPPSTFTQWLLQDPKFPQPHPERNKHSPDFSAFDACHNGASFFKLLQEPDSGIFPCQYKGPMFMTIGYVAVNYIAGIEIPEHERIELIRYIVNTAHPVDGGWGLHSVDKSTVFGTVLNYVILRLLGLPKDHPVCAKARSTLLRLGGAIGSPHWGKIWLSALNLYKWEGVNPAPPETWLLPYSLPMHPGRWWVHTRGVYIPVSYLSLVKFSCPMTPLLEELRNEIYTKPFDKINFSKNRNTVCGVDLYYPHSTTLNIANSLVVFYEKYLRNRFIYSLSKKKVYDLIKTELQNTDSLCIAPVNQAFCALVTLIEEGVDSEAFQRLQYRFKDALFHGPQGMTIMGTNGVQTWDCAFAIQYFFVAGLAERPEFYNTIVSAYKFLCHAQFDTECVPGSYRDKRKGAWGFSTKTQGYTVADCTAEAIKAIIMVKNSPVFSEVHHMISSERLFEGIDVLLNLQNIGSFEYGSFATYEKIKAPLAMETLNPAEVFGNIMVEYPYVECTDSSVLGLTYFHKYFDYRKEEIRTRIRIAIEFIKKSQLPDGSWYGSWGICFTYAGMFALEALHTVGETYENSSTVRKGCDFLVSKQMKDGGWGESMKSSELHSYVDSEKSLVVQTAWALIALLFAEYPNKEVIDRGIDLLKNRQEESGEWKFESVEGVFNHSCAIEYPSYRFLFPIKALGMYSRAYETLRLEQVLVLVLEQFCLKVKNYSLVLSQFWLNMVMLMVTNFLSPVKVKVMLLTVNPNLFVLLVNCQFHGQPSLLSVMVFNVLRDTQIINNMTFSSLPCQKVMFKKELFFSKMTVTTRPELKSSLKVIPLIESNKVLILKKMVTFVTNWNTTITLTMFTSWLTNKRMVSKLTSKLDTTLKMVLFNLTIINKILQLVMVQSCYQTTITYPLNLPYPKIQTKRETTWSCNLLLLLVLPMVWMNCTNCQCLVHKNLLLLCTRRLVLKTCTSNSTSLVLGSCQFLWLREGSISLIIKNSFRLGGGKEKLMREIAHFLMRNMLCSQKDIRYFLLQLVMFLFELQLIWIEFLQQVLVLVLEQCLNLKKICINYIWKVQLIIIILNVHLKVKVNHMKVHKQELKLLKVVHYHLLLIFLHLLCMVLRHLLIIHKVFQIFLNNLFQKVLHGKELQHMKMVVFQLHKIHLYKMVVFIMLKLEVLIFHLMVQLCKKKHVGKLLQKCYIQLMVVKVGLIWLNLVVVIFVIKQHIGLKNQLKIKCQVFIMLIEDKELKKLIKKHMLNNMKLLLLDIVIYHLNVIKDP

SEQ ID NO: 43 - DNA, 3983 bp, synthetic construct
  misc_feature 1..3983: note = ERG7 in expression cassette, derived from yeast
  source 1..3983: mol_type = other DNA
atgacagaattttattctgacacaatcggtctaccaaagacagatccacgtctttggagactgagaactgatgagctaggccgagaaagctgggaatatttaacccctcagcaagccgcaaacgacccaccatccactttcacgcagtggcttcttcaagatcccaaatttcctcaacctcatccagaaagaaataagcattcaccagatttttcagccttcgatgcgtgtcataatggtgcatcttttttcaaactgcttcaagagcctgactcaggtatttttccgtgtcaatataaaggacccatgttcatgacaatcggttacgtagccgtaaactatatcgccggtattgaaattcctgagcatgagagaatagaattaattagatacatcgtcaatacagcacatccggttgatggtggctggggtctacattctgttgacaaatccaccgtgtttggtacagtattgaactatgtaatcttacgtttattgggtctacccaaggaccacccggtttgcgccaaggcaagaagcacattgttaaggttaggcggtgctattggatcccctcactggggaaaaatttggctaagtgcactaaacttgtataaatgggaaggtgtgaaccctgcccctcctgaaacttggttacttccatattcactgcccatgcatccggggagatggtgggttcatactagaggtgtttacattccggtcagttacctgtcattggtcaaattttcttgcccaatgactcctcttcttgaagaactgaggaatgaaatttacactaaaccgtttgacaagattaacttctccaagaacaggaataccgtatgtggagtagacctatattacccccattctactactttgaatattgcgaacagccttgtagtattttacgaaaaatacctaagaaaccggttcatttactctctatccaagaagaaggtttatgatctaatcaaaacggagttacagaatactgattccttgtgtatagcacctgttaaccaggcgttttgcgcacttgtcactcttattgaagaaggggtagactcggaagcgttccagcgtctccaatataggttcaaggatgcattgttccatggtccacagggtatgaccattatgggaacaaatggtgtgcaaacctgggattgtgcgtttgccattcaatactttttcgtcgcaggcctcgcagaaagacctgaattctataacacaattgtctctgcctataaattcttgtgtcatgctcaatttgacaccgagtgcgttccaggtagttatagggataagagaaagggggcttggggcttctcaacaaaaacacagggctatacagtggcagattgcactgcagaagcaattaaagccatcatcatggtgaaaaactctcccgtctttagtgaagtacaccatatgattagcagtgaacgtttatttgaaggcattgatgtgttattgaacctacaaaacatcggatcttttgaatatggttcctttgcaacctatgaaaaaatcaaggccccactagcaatggaaaccttgaatcctgctgaagtttttggtaacataatggtagaatacccatacgtggaatgtactgattcatccgttctggggttgacatattttcacaagtacttcgactataggaaagaggaaatacgtacacgcatcagaatcgccatcgaattcataaaaaaatctcaattaccagatggaagttggtatggaagctggggtatttgttttacatatgccggtatgtttgcattggaggcattacacaccgtgggggagacctatgagaattcctcaacggtaagaaaaggttgcgacttcttggtcagtaaacagatgaaggatggcggttggggggaatcaatgaagtccagtgaattacatagttatgtggatagtgaaaaatcgctagtcgttcaaaccgcatgggcgctaattgcacttcttttcgctgaatatcctaataaagaagtcatcgaccgcggtattgaccttttaaaaaatagacaagaagaatccggggaatggaaatttgaaagtgtagaaggtgttttcaaccactcttgtgcaattgaatacccaagttatcgattcttattccctattaaggcattaggtatgtacagcagggcatatgaaacactacgcttggagcaggtgctggtgctggtgctggagcaattctgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaattgccaatgtcttgtgcacaagaatctattacttctttgtacaagaaggctggttctgaaaacttgtacttccaattccacaagtctggtgcttggaagttgccagtttctttggttaagagagggatcgataagcttgattataaagaacagcttcaggcttggcggtgggaaagagaaattgatgagagaaatcgcccactttctgatgaggaattagatgctatgttcccagaaggatataaggtacttcctcctccagctggttatgttcctattcgaactccagctcatatggatcgaattcctgcagtagcaggtgctggtgctggtgctggagcaatgtctgaattaattaaagaaaatatgcatatgaaattatatatggaaggtacagttaataatcatcattttaaatgtacatctgaaggtgaaggtaaaccatatgaaggtacacaaacaatgagaattaaagttgttgaaggtggtccattaccatttgcttttgatattttagctacatcttttatgtatggttctaagacatttattaatcatacacaaggtattccagatttttttaaacaatcttttccagaaggttttacatgggaaagagttacaacatatgaagatggtggtgttttaacagctacacaagatacatctttacaagatggttgtttaatttataatgttaaaattagaggtgttaattttccatctaatggtccagttatgcaaaaaaaaacattaggttgggaagcttctacagaaatgttatatccagctgatggtggtttagaaggtaggtctgatatggctttaaaattagttggtggtggtcatttaatttgtaatttgaaaacaacatataggtctaaaaaaccagctaaaaatttaaaaatgccaggtgtttattatgttgatagaagattagaaagaattaaagaagctgataaagaaacatatgttgaacaacatgaagttgctgttgctagatattgtgatttaccatctaaattaggtcataaaggatcctaa

SEQ ID NO: 44 - DNA, 3954 bp, synthetic construct
  misc_feature 1..3954: note = ERG7 in expression cassette, derived from yeast
  source 1..3954: mol_type = other DNA
ttccggatattgtgttaaaatttcaagtatgctactaatggggtaaagttgcatgattagcagagacatatggcttgttatggttcggcttcctcatttttcatgcttagtttttgtccatctcattgtacatttctgaatcctaatgcatgactccctaacattactattaaattctcaatagtgaagaataagcaaaatgggaaccatgataatttctagctttctctccacccctattttaatttgcaatcatatatagtactttcaatagcatcttttctagatttgatatctgcggaaacttttcgtttataatcgtttaggtgaaaagtttttatatcggttatttacagatatacattttctcaaaaaaaaaaaaatataatacatgccctcagcttttaataaagccattgagcacaagcctctccagtaatgtactgctgtgcccaataaccttaccaataatcgtcgcccacaaagaaagtacaaaacagatgacagaattttattctgacacaatcggtctaccaaagacagatccacgtctttggagactgagaactgatgagctaggccgagaaagctgggaatatttaacccctcagcaagccgcaaacgacccaccatccactttcacgcagtggcttcttcaagatcccaaatttcctcaacctcatccagaaagaaataagcattcaccagatttttcagccttcgatgcgtgtcataatggtgcatcttttttcaaactgcttcaagagcctgactcaggtatttttccgtgtcaatataaaggacccatgttcatgacaatcggttacgtagccgtaaactatatcgccggtattgaaattcctgagcatgagagaatagaattaattagatacatcgtcaatacagcacatccggttgatggtggctggggtctacattctgttgacaaatccaccgtgtttggtacagtattgaactatgtaatcttacgtttattgggtctacccaaggaccacccggtttgcgccaaggcaagaagcacattgttaaggttaggcggtgctattggatcccctcactggggaaaaatttggctaagtgcactaaacttgtataaatgggaaggtgtgaaccctgcccctcctgaaacttggttacttccatattcactgcccatgcatccggggagatggtgggttcatactagaggtgtttacattccggtcagttacctgtcattggtcaaattttcttgcccaatgactcctcttcttgaagaactgaggaatgaaatttacactaaaccgtttgacaagattaacttctccaagaacaggaataccgtatgtggagtagacctatattacccccattctactactttgaatattgcgaacagccttgtagtattttacgaaaaatacctaagaaaccggttcatttactctctatccaagaagaaggtttatgatctaatcaaaacggagttacagaatactgattccttgtgtatagcacctgttaaccaggcgttttgcgcacttgtcactcttattgaagaaggggtagactcggaagcgttccagcgtctccaatataggttcaaggatgcattgttccatggtccacagggtatgaccattatgggaacaaatggtgtgcaaacctgggattgtgcgtttgccattcaatactttttcgtcgcaggcctcgcagaaagacctgaattctataacacaattgtctctgcctataaattcttgtgtcatgctcaatttgacaccgagtgcgttccaggtagttatagggataagagaaagggggcttggggcttctcaacaaaaacacagggctatacagtggcagattgcactgcagaagcaattaaagccatcatcatggtgaaaaactctcccgtctttagtgaagtacaccatatgattagcagtgaacgtttatttgaaggcattgatgtgttattgaacctacaaaacatcggatcttttgaatatggttcctttgcaacctatgaaaaaatcaaggccccactagcaatggaaaccttgaatcctgctgaagtttttggtaacataatggtagaatacccatacgtggaatgtactgattcatccgttctggggttgacatattttcacaagtacttcgactataggaaagaggaaatacgtacacgcatcagaatcgccatcgaattcataaaaaaatctcaattaccagatggaagttggtatggaagctggggtatttgttttacatatgccggtatgtttgcattggaggcattacacaccgtgggggagacctatgagaattcctcaacggtaagaaaaggttgcgacttcttggtcagtaaacagatgaaggatggcggttggggggaatcaatgaagtccagtgaattacatagttatgtggatagtgaaaaatcgctagtcgttcaaaccgcatgggcgctaattgcacttcttttcgctgaatatcctaataaagaagtcatcgaccgcggtattgaccttttaaaaaatagacaagaagaatccggggaatggaaatttgaaagtgtagaaggtgttttcaaccactcttgtgcaattgaatacccaagttatcgattcttattccctattaaggcattaggtatgtacagcagggcatatgaaacactacgcttggagcaggtgctggtgctggtgctggagcaattctgtctaaaggtgaagaattattcactggtgttgtcccaattttggttgaattagatggtgatgttaatggtcacaaattttctgtctccggtgaaggtgaaggtgggatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagggcgagctcgaattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcgacccggacgggacgctcctgcgcctgatacagaacgaattgcttgcaggcatctcatgagtgtgtcttcccgttttccgcctgaggtcactgcgtggatggagcgctggcgcctgctgcgcgacggcgagctgctcaccacccactcgacggatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcccaat

SEQ ID NO: 45 - protein, 759 aa, synthetic construct; identical to SEQ ID NO: 19
  REGION 1..759: note = yeast modified

SEQ ID NO: 46 - DNA, 2280 bp, synthetic construct
  misc_feature 1..2280: note = yeast modified
  source 1..2280: mol_type = other DNA
atgacagaattttattctgacacaatcggtctaccaaagacagatccacgtctttggagactgagaactgatgagctaggccgagaaagctgggaatatttaacccctcagcaagccgcaaacgacccaccatccactttcacgcagtggcttcttcaagatcccaaatttcctcaacctcatccagaaagaaataagcattcaccagatttttcagccttcgatgcgtgtcataatggtgcatcttttttcaaactgcttcaagagcctgactcaggtatttttccgtgtcaatataaaggacccatgttcatgacaatcggttacgtagccgtaaactatatcgccggtattgaaattcctgagcatgagagaatagaattaattagatacatcgtcaatacagcacatccggttgatggtggctggggtctacattctgttgacaaatccaccgtgtttggtacagtattgaactatgtaatcttacgtttattgggtctacccaaggaccacccggtttgcgccaaggcaagaagcacattgttaaggttaggcggtgctattggatcccctcactggggaaaaatttggctaagtgcactaaacttgtataaatgggaaggtgtgaaccctgcccctcctgaaacttggttacttccatattcactgcccatgcatccggggagatggtgggttcatactagaggtgtttacattccggtcagttacctgtcattggtcaaattttcttgcccaatgactcctcttcttgaagaactgaggaatgaaatttacactaaaccgtttgacaagattaacttctccaagaacaggaataccgtatgtggagtagacctatattacccccattctactactttgaatattgcgaacagccttgtagtattttacgaaaaatacctaagaaaccggttcatttactctctatccaagaagaaggtttatgatctaatcaaaacggagttacagaatactgattccttgtgtatagcacctgttaaccaggcgttttgcgcacttgtcactcttattgaagaaggggtagactcggaagcgttccagcgtctccaatataggttcaaggatgcattgttccatggtccacagggtatgaccattatgggaacaaatggtgtgcaaacctgggattgtgcgtttgccattcaatactttttcgtcgcaggcctcgcagaaagacctgaattctataacacaattgtctctgcctataaattcttgtgtcatgctcaatttgacaccgagtgcgttccaggtagttatagggataagagaaagggggcttggggcttctcaacaaaaacacagggctatacagtggcagattgcactgcagaagcaattaaagccatcatcatggtgaaaaactctcccgtctttagtgaagtacaccatatgattagcagtgaacgtttatttgaaggcattgatgtgttattgaacctacaaaacatcggatcttttgaatatggttcctttgcaacctatgaaaaaatcaaggccccactagcaatggaaaccttgaatcctgctgaagtttttggtaacataatggtagaatacccatacgtggaatgtactgattcatccgttctggggttgacatattttcacaagtacttcgactataggaaagaggaaatacgtacacgcatcagaatcgccatcgaattcataaaaaaatctcaattaccagatggaagttggtatggaagctggggtatttgttttacatatgccggtatgtttgcattggaggcattacacaccgtgggggagacctatgagaattcctcaacggtaagaaaaggttgcgacttcttggtcagtaaacagatgaaggatggcggttggggggaatcaatgaagtccagtgaattacatagttatgtggatagtgaaaaatcgctagtcgttcaaaccgcatgggcgctaattgcacttcttttcgctgaatatcctaataaagaagtcatcgaccgcggtattgaccttttaaaaaatagacaagaagaatccggggaatggaaatttgaaagtgtagaaggtgttttcaaccactcttgtgcaattgaatacccaagttatcgattcttattccctattaaggcattaggtatgtacagcagggcatatgaaacactacgcttggagcaggtgctggtgctggtgctggagcaattctgtctaaaggtgaagaattattcactggtgttgtcccaattttggttgaattag